SEQUENCE LISTING (69 sequences)

SEQ ID NO: 1 - protein, 770 aa, Prevotella histicola
MNLKTFTKTVLCFALFAVSAITAKAADHLAIVGEAVWGGWDLVKATAMVKSPNNPDVFMATVHLNAGKGFKFLTEREWGKLEYRSGASDVVLKSGIRYKLYASIGASEDGKFKVSESANYEIICDLARKTVEVKKVAYQAKEIRYAALWMIGDATAGDWDYNNGVLLSQDSGNPTCYTATVELKEGEFKFTTNKQWGYDHSVYIFRDVNDQNKIVFGGEDNKWRITEDGMYNVTVDVPTKTISIKQIDDPAGHKPQFGNDVILVGDATIAGWNLDNAIYLEHTGQAGRVFKTTTYLEAGKGFKFLSMLSYDDIDYRPANNTVLNPGVPGTFVPSLPSSTDTKFSVERSGNYDIVCNMNNRTVVVTLSENQVLVNYPALWLIGSATSAGWNPGKAVELKRSEADPAVYTARVQLKKGEFKILTSKNVGFDQPTYYRDSTNEHRIVFGVDGDEVAKKDCKWTLSENAEGTYDVTVDIEAMTIFCDKVNMDEPSVESTDKELILIGDATYSAWDLPKSIVMTPVGPTTFKAVTHLEAGKEFKFLTELAWKRYEYRAESLRKELQEGSMSMLVPYRYTNDKDDKDHDFKFVVKESGNYEIVCDLYIPALIIRKVRYQDTPVTYSSLWIVGSATPGGWTIERGIKMTQDENYPTKFTAKANLVPGELKFATNKFADFTQDFFFRGKDDYTAVLGGNDNKWNITEAGTYSVTIDVASKRVTITKPARNAPTGISTVDSSDEAPAEYFTLNGIKVTTPSSGIYIKRQGGRTTKVVMK

SEQ ID NO: 2 - protein, 193 aa, Salmonella enterica
MDTYQILDIIGCIVGLIYIYQEYKASIWLWMTGIIMPVIYMFVYYEAGLYADFGMQIYYTLAAIYGYLYWKLGKKKGTEDKEIPITHFPRRYIIPAIIVFFVLWIALYYILICFTNSTVPVLDSFGNALSFIGLWALAKKYLEQWWIWIVVDAELSALYIYKGIPFTAMLYALYTVIAVAGYFKWRRYIKQQK

SEQ ID NO: 3 - protein, 544 aa, Bacillus licheniformis
MRVRLYKNILLFLFLWVNTLACVSADTSRTVESQPIENGLIITESKGWLETIYAKWKPVAEADGYYVYVKGGQYADYSKVDSELIRVYNGYVRVDIPGLKAGTYSLKIVAVKGGKETQSSEVTGLKVLNYVREGFAHKNYSGVGAYNDDGTLKSGAVVIYVNKDNAKTVSAHLGKTTFIGLQAILNAYQKGNITTPLSVRILGLLRNGDTDTFGSSTEGIQIKGKQADSEMNITIEGIGEDASIYGFGFLVRNAKSVEFRNLGIMRAMDDGVSLDTNNSNIWIHHMDLFYGKASGGDHIKGDGSIDVKTDSKYVTIDNCHFWDTGKTSMCGMKKETGPNYITYHHNWFDHSDSRHARVRTMSVHLWNNYYDGCAKYGIGATMGCSVFSENNYFRATKNPILISKQGSDAKGTGKFSGEPGGMVKEYGSLFTEKGAESTYTPISYADNNSSFDFYHAISRNEKVPASVKTLNGGNIYNNFDTDAALMYSYTPDATALVPSQVTGFYGAGRLNHGSLQFKFNNAVEDTNSTPIPALEALIDAYSGK

SEQ ID NO: 4 - protein, 366 aa, Streptococcus gordonii
MKYNIAYCIEGFYNHGGMERILSVCANLLSDIYSITIIVANQRGREHAYNLAQNVNVVDLGVSCKNYKEEYKKSLTRYLQDHQFSVVISLAGLELFFLPQIKDGSKKVMWFHFAFDVSKMFLSERFHGWKLNLLYYIHTIRRIYFAKKFDTIVVLSKSDCDSWSRFCNNVKYIYNPITIDRKVISNLSEESVIAVGRLGWQKGFDFLIDSWVLVDDKHPDWHLDIFGEGPDRLELQHQIDRKGLHDKVRLCGVTKQIEEEYGKHSIYVMSSRAEGFPLALLEASSCGLPMISFNCHQGPNEIIQEGENGFLVDKVGDIYTLSDRICKLIEDNNLRNMMGKKALDSSFRFEGEVIKKDWISLLKQLI

SEQ ID NO: 5 - protein, 158 aa, Prevotella histicola
MKRLFFMFLFLGTITMNSLAQEEKPIKYETKNFSLPDKMPLYPGGDGALRAFLSLNLHYPEKAQAFGVEGRSLMKFCVSSDGSIKDISAVDCKITNYNRTEFNKLPLSKQESLKKECAKAFAKEAARVIRLMPKWEPAELNGKKMNVYYSLPFTFKLR

SEQ ID NO: 6 - protein, 415 aa, Prevotella histicola
MNYPLFIARKIYNGGDRTRKVSKPAIRIATIGVAIGLAVMIISVGVVLGFKHTIRNKVVGFGSDITVANFLTLQSSEQYPIQITDSLVKSLQITPGIKHVQRYDYTQGILKTDNDFLGVLLKGVGPDFDSTFIHENMVEGSLPHFHDNESQQKIVISKTIADKLNLKVGQRIFAYFINKQGVRTRKFTITGIYATNMKQFDSQICFTDIYTTNKLNGWEPDQYSGAELQVDNFSQLTPISMRVLNKVKNTVDHYGGTYSSENIIEQNPQIFSWLDLMDMNVWIILALMISVAGVTMISGLLIIILERTQMIGILKALGSRNRQIRHIFLWFATFIIGKGLLWGNIIGLGCILFQSWTGLVKLDPQTYYVNTVPVEINIPLIIALNMVTMLVCLVILIAPSYLISHIHPAKSMHYE

SEQ ID NO: 7 - protein, 736 aa, Mycobacterium tuberculosis
MEDKFIYTDKERKLSYQILDELKDTLDKSFLENDLPMLQVQLKDSVAKNTIHRNVFGLNPILCSLQTAAIAVKDIGLKRDSVIAILLHQSVQDGYITLEDIDNRFGKSVAKIIHGLIRIQTLYQKNPIIESENFRNLLLSFAEDMRVILIMIADRVNLMRQIRDAEDKEAQHKVAEEASYLYAPLAHKLGLYQLKRELEDLSLKYLEHDAYYLIKDKLNATKASRDAYINQFIAPVRERLTAGGLRFHIKGRTKSIHSIWQKMKKQKCGFEGIYDLFAIRIILDAPLEKEKIQCWQAYSIVTDMYQPNPKRLRDWLSVPKSNGYECLHITVLGPEKKWVEVQIRTERMDEIAEHGLAAHWRYKGIKEEGGLDDWLASIRAALEAGDNLEVMDQFKSDLYEKEIYVFTPKGDLLKFPKGATILDFAYHIHSKVGNQCVGGKINAKNVSLRTELHSGDTVEILTSATQKPKAEWLKIVKSSRAKAKIRLALKETQIKDGLYAKELLERRFKNKKIEIEESTMGHLLRKLGFKEVSEFYKQVADEKLDPNYIIEEYQKVYNHDHNLNQPKETESAENFEFENPTNEFLKKNDDVLVIDKNLKGLDFSLAKCCHPIYGDPVFGFVTVNGGIKIHRTDCPNAPEMRKRFGYRIVKARWSGKGSSQYAITLRVIGNDDIGIVSNITNVISKDEKIVMRSINIDSHDGLFSGNLVVLLDDNSKLNMLIKKLRTVKGVKQVTRI

SEQ ID NO: 8 - protein, 336 aa, Escherichia coli
MKRRIFLFVALSVSIVILFGLNLIIGSVHIPLSDILTILSGSFTGKESWRFIIWDSRLPQALTAMLCGSSLAVCGLMLQTAFRNPLAGPDVFGISSGASLGVALVMLLLGGTVETSMFTASGFLAILIVAFAGAILVTAFILFLSSVVRNSVLLLIVGIMVGYVASSAVTLLNFFSSEDGVKGYIVWGMGNFGGVSMSHIPLFAFLCLAGIIASFLLVKPLNILLLGPQYAESLGISIRRIRNILLVVVGILTAVTTAFCGPISFIGLAAPHVARLLFRTENHQKLLPGTLLVGTVVALLCNLICFLPRESGMIPLNAVTPLIGAPIIIYVIMKRH

SEQ ID NO: 9 - protein, 524 aa, Escherichia coli
MKLENKEFGFDSFATEMARLKNEKHFDYLVTVVGEDFGTEEGLGCIYILENTSTHERCSVKQLAKKVGEEFVIPSVIKLWADADLLEREVYDFYGIKFLGHPDMRRLFLRNDFKGYPLRKDYDMDPAKNMYTTEDDVELDTTTEWNLDKNGELVGTQHALFTDDNFVVNIGPQHPSTHGVLRLQTVLDGETVTNIYPHLGYIHRGIEKLCEQFTYPQTLALTDRMNYLSAMMNRHALVGVIEEGMGIELSERILYIRTIMDELQRIDNHLLYTACCAQDLGALTAFLYGMRDREHVLNVMEETTGGRLIQNYYRIGGLQADIDPNFVSNVKELCKYLRPMIQEYVDVFGDNVITHQRFEGVGVMDEKDCISYGVTGPAGRASGWKNDVRKYHPYAMYDKVNFEEITLTNGDSMDRYFCHIKEIYQSLNIIEQLIDNIPEGEFYIKQKPIIKVPEGQWYFSVEGASGEFGAYLDSRGDKTAYRLKFRPMGLTLVGAMDKMLRGQKIADLVTTGAALDFVIPDIDR

SEQ ID NO: 10 - protein, 142 aa, Escherichia coli
MRTSTQSKDMGKKQEYKLRNEEFLHNISKKDSIKTLPHGIFYEIIKEGSGEGTVQPRSIVICNYRGSLISGQVFDDSWQKPTPEAFRLNELITGLQIALCAMHKGDSWRIYIPYQEGYGSKRNADIPAFSTLIFDIELINIA

SEQ ID NO: 11 - protein, 196 aa, Mycobacterium tuberculosis
MADNKIAKESVKREVIAGERLYTLLVYSENVAGVLNQIAAVFTRRQVNIESLNVSASSIEGIHKYTITAWSDAATIEKITKQVEKKIDVIKADYYEDSDLFIHEVGLYKIATPILLENAEVSRAIRKRNARMMEVNPTYSTVLLAGMTDEVTALYHDLKNFDCLLQYSRSGRVAVTRGFSEPVSDFLKSEEESSVL

SEQ ID NO: 12 - protein, 390 aa, Escherichia coli
MKKKVKIGLLPRVIIAILLGIFFGYFMPTPLARVFLTFNGIFSQFLGFMIPLIIIGLVTPAIADIGKGAGKLLLVTVIIAYVDTVVAGGLAYGTGLCLFPSMIASTGGAMPHIDKATELAPYFSINIPAMADVMSGLVFSFMLGLGIAYGGLTATKNIFNEFKYVIEKVIAKAIIPLLPLYIFGVFLNMAHNGQAQQILLVFSQIIIVILVLHVFILVYQFCIAGAIIRRNPFRLLWNMMPAYLTALGTSSSAATIPVTLEQTMKNGVGKEIAGFVVPLCATIHLSGSAMKITACALTICLLVGLPHDPALFIYFILMLSIIMVAAPGVPGGAIMAALAPLASILGFNSEAQALMIALYIAMDSFGTACNVTGDGAIALVVNKMFGKKER

SEQ ID NO: 13 - protein, 162 aa, Prevotella histicola
MKKLLLLVCAAVMSLSASAQAGDKALGAQLVFGSETNSLGFGVKGQYYFTDHIRGEGSFDYFLKNKGISMWDINANVHYLFDVADKFKVYPLAGLGYTNWSYKYEYAGAPVVEGSDGRLAVNLGGGVEYELTKNLNVNAEAKYQIISNYNQLVLGVGVAYKF

SEQ ID NO: 14 - protein, 84 aa, Nostoc sp.
MHFYCTKSSLDTMSERYVKRMIAKLASQGKTVISIAHRFSTIMDAKHIILLAKGKVVAEGTHQELLKTSEDYRKLWSDQNDEID

SEQ ID NO: 15 - protein, 254 aa, Pseudomonas aeruginosa
MKNVYFLSDAHLGSLAIAHRRTQERRLVRFLDSIKHKASAVYLLGDMFDFWDEYKYVVPKGFTRFLGKVSELTDMGVEVHFFTGNHDLWTYGYLEEECGVILHRKPVTMEIYGKVFYLAHGDGLGDPDPMFQFLRKVFHNRVCQRLLNFFHPWWGMQLGLNWAKKSRLKRADGKEMPYLGEDKEYLVRYTKDYMRSHKDIDYYIYGHRHIELDLTLSGKVRMLILGDWIWQFTYAVFDGEHMFLEEYIEGESKP

SEQ ID NO: 16 - protein, 532 aa, Escherichia coli
MNSKQNDNYDVIIIGGGITGAGTARDCALRGLKVLLVEKFDFTNGATGRNHGLLHSGARYAVTDPESATECIKENMVLRRIAKHCIEETDGLFITLPEDDINYQKTFVEACARAGISANIISPEEALRLDPSVNPDLLGAVRVPDASVDPFHLTTANVLDARQHGADVLTYHEVVAILTSNGRVEGVRLRNNHTGEEIEKHAVLVINAAGIWGHDIAKMADIKINMFPAKGTLLVFGHRVNKMVINRCRKPANADILVPDDAVCVIGTTSDRVPYDTVDNLKITSEEVDTLIREGEKLAPSLATTRILRAYAGVRPLVAADNDPTGRSISRGIVCLDHEKRDGLTGMITITGGKMMTYRLMAEQATDLACKKLGINKTCETATTPLPGTAGKDSDNPHHTYSTAHKAAKGRQGNRVKEIDERTEDDRALICECEEVSVGEAKYAIEELHVHDLLNLRRRTRVGMGTCQGELCACRAAGVMCENGVKVDKAMTDLTKFINERWKGMRPVAWGSTLDEAQLTTIIYQGLCGLGI

SEQ ID NO: 17 - protein, 416 aa, Escherichia coli
MRYDTIIIGGGLSGLTAGITLAKAGQKVCIVSAGQSSLHFHSGSFDLLGYDADGEVVTHPLQAIADLKAEHPYSKIGISNIEHLASQAKTLLCEAGISVMGNYEQNHYRVTPLGTLKPAWLTTEGYAMIDDPEILPWKKVELLNIQGFMDFPTQFIAENLRMMGVECQIKTFTTDELSTARQSPTEMRATNIAKVLANKDALSKVSERINAISGDPDALLLPAVLGFSNAESLDEMKQWIKKPVQYIATLPPSVSGVRTTILLKRLFAQAGGTLLIGDSATTGQFSGNHLVSITTDHLPDEKLYADHFILASGSFMSHGIRSNYAGVYEPVFKLDVDAAEKRDDWSVTNAFEAQPYMEFGVHTDKDFHATKDGKNIENLYAIGSVLSGHNSIKHADGTGVSLLTALYVAKKITGKG

SEQ ID NO: 18 - protein, 416 aa, Escherichia coli
MAEGIQLKNISGNNLEQCLKCSICTAYCPVSAVEPKYPGPKQSGPDQERYRLKDSKFFDEALKMCLNCKRCEVACPSGVRIADIIQASRITYSTHRPIPRDIMLANTDFVGTMANMVAPIVNATLGLKPVKAVLHGVMGIDKHRTFPAYSSQKFETWYKRMAAKKQDSYSKHVSYFHGCYVNYNFPQLGKDLVKIMNAVGYGVHLLEKEKCCGVALIANGLSGQARRQGKVNIRSIRKAAEQNRIVLTTSSTCTFTMRDEYEHLLDIKTDDVRENITLATRFLYRLIEKGDIKLAFRKDFKMRTAYHSACHMEKMGWIIYSTELLKMIPGLELIMLDSQCCGIAGTYGFKKENYQRSQEIGEGLFKQIKELNPDCVSTDCETCKWQIEMSTGYEVKNPISILADALDVEETIKLNQ

SEQ ID NO: 19 - protein, 270 aa, Bacillus subtilis
MMIKNIVLSIPISLIIYLNHLIMEYSMTTQFLMELIGTLILVLFGDGVCACVTLNKSKGQKAGWVVITIAWGLAVCMGVLVAGPYTGAHLNPAVSIGLAVAGMFPWSSVPYYIVAQMIGGFLGGLLVWFFYKDHYDATDDEAAKLGTFCTSPAIRNYKMNFLSEVIATLVLVFIIISFSVDGNTGDAEHFKFGLAALGPIPVTLLIIALGMSLGGTTGYAMNPARDLSPRLAHAVCMKGDNDWSYSWIPVLGPIIGAIIAGFCGAALLLV

SEQ ID NO: 20 - protein, 398 aa, Streptococcus pneumoniae
MSEKIIPSNEPAQAASEPIKASYTEYTVIPSQGYCQFVKCKKGDQPVVLKGLKEAYRERVLLRNALKREFKQCQRLNHPGIVRYQGLVDVEGYGLCIEEEYVDGRTLQAYLKESHTDDEKITIVNQIADALRYAHQQGVAHRNLKPSNILITKQGDHVKLIDFNVLSLDDVKPTADTTRFMAPELKDETMTADGTADIYSLGTIMKVMGLTLAYSEVIKRCCAFKRSDRYSDIDEFLADFNHDGSSFSMPKIGKGTVVIGFIAVVVIALAALAYNYGGALVDQVGKIDVTSIFKSDAETAPEDSAMVKSVEQNNNDSVADEAPATGKLAFMNTMKPALYKDLDRLFAKHSDDRAKLNRAIKVYYRGLIQANDTLDNEQRAELDRVFGNYVKQKKAALK

SEQ ID NO: 21 - protein, 62 aa, Prevotella histicola
MLVAQLFVGVLQAQKPVQNRRQAVGQSMERQGLVNVKAVVPSIKVALMYARTDNFCHRMALS

SEQ ID NO: 22 - protein, 440 aa, Escherichia coli
MITGLVIIQLLIVLALIFIGARVGGIGLGIYGMIGVFILVYGFGLAPGSAPIDVMMIIVAVITAASALQASGGLEYLVGVAAKFLQKHPDHITYFGPITCWLFCVVAGTAHTSYSLMPIIAEIAQTNKIRPERPLSLSVIAASLGITCSPVSAATAALISQDLLGAKGIELGTVLMICIPTAFISILVAAFVENHIGKELEDDPEYKRRVAAGLINPEAACEEVQKAENEHDPSAKHAVWAFLFGVALVILFGFLPQLRPEGVSMSQTIEMIMMSDAALILLVGKGKVGDAVNGNIFKAGMNAVVAIFGIAWMGNTFYVGNEKILDAALSSMISSTPILFAVALFLLSIMLFSQAATVTTLYPVGIALGINPLLLIAMFPACNGYFFLPNYPTEVAAIDFDRTGTTRVGKYVINHSFQIPGFITTIVSILLGVLMVQFFR

SEQ ID NO: 23 - protein, 351 aa, Escherichia coli
MRILKITFVTVLALVMSTVVFAQKPKIRIIATGGTIAGVSASATSSAYGAGQVGVQTLIDAVPQIKDIADVSGEQLVNIGSQDMNDEVWLKLAKRINDLLNKEGYDGVLITHGTDTMEETAYFLSLTVHTDKPVVMVGSMRPSTAISADGPANLYNGICTLVDPSSKGHGVMVCMNNELFEAKSVIKTHTTDVSTFKGGLYGEMGYVYNGKPYFLHKPVAKQGLTSEFNVDNLTSLPKVGIVYGYANCSPLPIQAFVNAKFDGIVLAGVGDGNFYKDVFDVALKAQNSGIQIVRSSRVPFGPTNLNGEVDDAKYHFVASLNLNPQKARVLLMLALTKTKDWQKIQQYFNEY

SEQ ID NO: 24 - protein, 574 aa, Mycobacterium tuberculosis
MALACAMTMSASAQMGTNPKWLGDAIFYQIYPSSYMDTDGNGIGDLPGITQKLDYIKSLGVNAIWLNPVFESGWFDGGYDVIDFYKIDPRFGTNTDMVNLVKEAHKRGIKVCLDLVAGHTSTKCPWFKESANGDRNSRYSDYFIWTDSISEADKKEIAERHKEANPASSTHGRYVEMNAKRGKYYEKNFFECQPALNYGFAKPDPNQPWEQPVTAPGPQAVRREMRNIMAFWFDKGVDGFRVDMASSLVKNDWGKKEVSKLWNEMREWKDKNYPECVLISEWSDPAVAIPAGFNIDFMIHFGIKGYPSLFFDRNTPWGKPWPGQDISKDYKFCYFDKAGKGEVKEFVDNFSEAYNATKNLGYIAIPSANHDYQRPNIGTRNTPEQLKVAMTFFLTMPGVPFIYYGDEIGMKYQMDLPSKEGSNERAGTRTPMQWTSGPTAGFSTCNPSQLYFPVDTEKGKLTVEAQQNDPRSLLNYTRELTRLRHSQPALRGNGEWILVSKESQPYPMVYKRTSGGETVVVAINPSDKKVSANIAHLGKAKSLIMTGKASYKTGKTEDAVELNGVSAAVFKIAE

SEQ ID NO: 25 - protein, 244 aa, Listeria monocytogenes
MNIAVIFAGGSGLRMHTKSRPKQFLDLNGKPIIIYTLELFDNHPGIDAIVVACIESWIPFLEKQLRKFEINKVVKIVPGGESGQASIYNGLCAAEAYIKSKNVASEDTTVLIHDGVRPLITEETITDNINKVAEVGSCITCIPATETLVVKQHDGSLEIPSRADSLIARAPQSFLLSDILTAHRRAIDEKKNDFIDSCTMMSHYGYRLGTIIGPMENIKITTPTDFFVLRAMVKVHEDQQIFGL

SEQ ID NO: 26 - protein, 327 aa, Escherichia coli
MTEKKSVSIVLCTYNGTKYLQEQLDSILAQTYPLHEIIIQDDGSTDNTWQILEKYEEKYPLIHIYHNEGTHGVNANFLSAMHRTTGDFIAIADQDDIWETDKIANQMTTIGNKLLCSGLTRPFSSDGSFAYFDNRPRNVSIFRMMFLGLPGHTMLFRRELLRMMPPVTHSFFNVSLYDAALSILAASHDSIAFCNKVLVNFRRHADATTYNDYSRSLPSWQNGLYELLWGLRHYHQARSIALPIYRGKLALMEGITTNYHDFIEAKAIMRLETQKGLWAFLRLQYLLTKNHQRLFQTSGGSFIKMIRAWLYPVMQLYMYHHALRRCK

SEQ ID NO: 27 - protein, 437 aa, Enterobacter cloacae
MESFIIEGGHRLSGTIAPQGAKNEALEVICATLLTTEEVIIRNIPNILDVNNLIKLLQDIGVKVKKLGANDFSFQADEVKLDYLESIDFVKKCSSLRGSVLMIGPLLGRFGKATIAKPGGDKIGRRRLDTHFLGFKNLGARFVRIEDRDVYEIQADKLVGDYMLLDEASVTGTANIIMSAVMAEGTTTIYNAACEPYIQQLCHLLNAMGAKITGIASNLITIEGVTSLHGAEHRILPDMIEVGSFIGMAAMVGDGVRIKDVSIPNLGLILDTFRRLGVQIIEDEDDLIIPRQDHYVIDSFIDGTIMTISDAPWPGLTPDLISVLLVVATQAQGSVLFHQKMFESRLFFVDKLIDMGAQIILCDPHRAVVVGHDHAKKLRAGRMSSPDIRAGIALLIAALTAEGTSRIDNIAQIDRGYENIEGRLNALGAKVQRVEIC

SEQ ID NO: 28 - protein, 772 aa, Escherichia coli
MERSGNFYKAIRLGYILISILIGCMAYNSLYEWQEIEALELGNKKIDELRKEINNINIQMIKFSLLGETILEWNDKDIEHYHARRMAMDSMLCRFKATYPAERIDSVRHLLEDKERQMCQIVQILEQQQAINDKITSQVPVIVQKSVQEQPKKSKRKGFLGIFGKKEEAKPTVTTTMHRSFNRNMRTEQQAQSRRLSVHADSLAARNAELNRQLQGLVVQIDGKVQTDLQKREAEITAMRERSFIQIGGLTGFVILLLVISYIIIHRNANRIKRYKQETADLIERLQQMAKRNEALITSRKKAVHTITHELRTPLTAITGYAGLIQKNFNADKTGMYIRNIQQSSDRMREMLNTLLSFFRLDDGKEQPNFSTCRISSIAHTLESEFMPIAINKGLALTVTNHTDAVVLTDKERILQIGNNLLSNAIKFTENGAVSLTMGYDNGMLKLIVKDTGSGMTEEEQQRVFGAFERLSNAAAKDGFGLGLSIVQRIVTMLGGTIQLKSEKGKGSRFTVEIPMQSAEELPERINKTQIHHNRTLHDIVAIDNDKVLLLMLKEMYAQEGIHCDTCTNAAELMEMIRRKEYSLLLTDLNMPDINGFELLELLRTSNVGNSRIIPIIVTTASGSCNREELLERGFSDCLLKPFSISELMEVSDKCAMKGKQNEKPDFSSLLSYGNESVMLDKLIAETEKEMQSVRDGEQRKDFQELDALTHHLRSSWEILRADQPLRELYKQLHGSAVPDYEALNNAVTAVLDKGSEIIRLAKEERRKYENG

SEQ ID NO: 29 - protein, 321 aa, Staphylococcus aureus
MKRSRFYITVGLILSLTLLMSACGQKKAKDGRTDTPTSGTIKFASDESFSPIVEELLQNYQFRYPQAHLLPIYTDDNTGMKLLLDQKVNLFITSHAMTKGEDAILRGKGPIPEVFPIGYDGIAFIVNRSNPDSCITVDDVKKILQGKIAKWNQLNPKNNRGSIEVVFDNKASATLHYVVDSILGGKNIKSENIVAAKNSKSVIDYVNKTPNAIGVIGSNWLNDHRDTTNTTFKKDVTVASISKATVASPSNSWQPYQAYLLDGRYPFVRTIYALLADPHKALPYAFANYIANPIGQMIIFKAGLLPYRGNINIREVEVKNQ

SEQ ID NO: 30 - protein, 198 aa, Mycobacterium tuberculosis
MAGTKRIKTALISVFHKDGLDDLLKKLDEEGVQFLSTGGTQQFIESLGYECQKVEDVTSYPSILGGRVKTLHPKIFGGILARRDNEEDQKQMVEYTIPAIDLVIVDLYPFEQTVASGASAQDIIEKIDIGGISLIRAGAKNFKDVVIVPSKAEYPVLLQLLNTKGAETEIEDRKMFAERAFGVSSHYDTAIHSWFAAE

SEQ ID NO: 31 - protein, 472 aa, Escherichia coli
MEEEKGGRIGQRPYILKIITERNYIIIIDMKKAKILLFVTALVAVLTSCGGGQKGLPTSDEYPVITIGASNAQLKTTYPATIKGVQDVEVRPKVSGFITKLNIHEGEYVHAGQVLFVIDNSTYQAAVRQAQAQVNSAQSAVAQAKANVVQANASLNSANAQAATSRLTYNNSQNLYNNKVIGDYELQSAKNTYETAQASVRQAQSGIASAQAAVKQAEAGVRQAQAMLSTAKDNLGFCYVKSPASGYVGSLPFKEDALVSASSAQPVTTISNTSTIEVYFSMTEADVLKLSRTDDGLSNAIKKFPAVSLLLADGSTYNHEGAIVKTSGMIDATTGTINVIARFPNPEHLLKSGGSGKIVIAKNNNRALLIPQEAVTQVQNKMFVYKVDAKDKVHYSEITVDPQNDGINYIVTSGLKMGERIVSKGVSSLEDGAKIKALTPAEYEEAIKKAEKLGENQSSASGFLKTMKGDSK

SEQ ID NO: 32 - protein, 292 aa, Bacillus anthracis
MAKRRNKARSHHSLQVVTLCISTAMVLILIGMVVLTVFTSRNLSSYVKENLTVTMILQPDMSTEESAALCQRIRSLHYINSLNFISKEQALKEGTRELGANPAEFAGQNPFTGEIELQLKANYANNDSIKNIERELRTYRGVSDITYPQNLVESVNHTLGKISLVLLVIAILLTIVSFSLMNNTIRLSIYARRFSIHTMKLVGASWGFIRAPFLRRAVMEGLVSALLAIAVLGVGLCLLYDYEPDITKVLSWDVLVITAGVMLAFGVLIATFCSWLSVNKFLRMKAGDLYKI

SEQ ID NO: 33 - protein, 828 aa, Campylobacter jejuni
MKLSDLKTGETGVIVKVLGHGGFRKRIIEMGFIQGKQVEVLLNAPLRDPVKYKIMGYEVSLRHSEADQIEVISAEEARQLEQAKADNEPQQGALSNNIPDESDHALTPFELTDAANRKSKVINVALVGNPNCGKTSLFNFASGAHERVGNYSGVTVDAKVGRANYEGYEFHLVDLPGTYSLSAYSPEELYVRKQLVEKTPDVVINVIDASNLERNLYLTTQLIDMHVRMVCALNMFDETEQRGDNIDYQKISELFGIPMVPTVFTNGRGVKELFHQVIAVYEGKEDETSQFRHIHINHGHELEGGIKNIQEHLRAYPDICQRYSTRYLAIKLLEHDKDVEELIKPLKDSDEIFKHRDIAAQRVKEETGNESETAIMDAKYGFIHGALEEADYSTGQKKDTYQTTHFIDQILTNKYFGFPIFFLILFIMFTATFVIGQYPMDWIDGGVSWLGDFISSNMPDGPVKDMLVDGIIGGVGAVIVFLPQILILYFFISYMEDSGYMARAAFIMDKLMHKMGLHGKSFIPLIMGFGCNVPAVMATRTIESRRSRLVTMLILPLMSCSARLPIYVMITGSFFALKYRSLAMLSLYVIGILMSVIMSRVFSRFLVKGEDTPFVMELPPYRFPTWKAIGRHTWEKGKQYLKKMGGIILVASIIVWALGYFPLPDKPDMGQQERQEHSFIGQIGHAVEPVFRPQGFNWKLDVGLLAGVGAKEIVASTMGVLYSNDDSFKDDNSFSSEGGKYVKLHKQITQDVANLHGVSYNEAEPIATLTAFCFLLFVLLYFPCIATIAAIKGETGSWGWALFAAGYTTLLAWVVSAIVFQVGMLFIG

SEQ ID NO: 34 - protein, 566 aa, Streptococcus pneumoniae
MKKNLLKAVLPASLALFAVTFGSCSQDGQLTGTKEDTGERVLDNTREIQNYLRTLPLAPMMSRASDPVPSDDGTTVPVDEGTSKTEEKGVLNGIPGSWVKTTRRYKMTQAFDESFLFDPTSDIVYPGCVLKGGTIANGTYAIITSHETGDVTFSINLSPANPQEARETSATVHNIRKSEYQEVWNKWANMQWKESPITTIESVEKINSQEELATKLGVAVNSPVANGSLNFGFNFNKKKNHILARLIQKYFSVSTDAPKKGNIFESIDKEALDGYQPVYISNINYGRIIYLSVESDEDEKVVDEAINFAMNQIKGVDVSVSADQSLHYRKVLANCDIRITVLGGGQTIQKEVLKGDIDSFQRFLNADIPMEQMSPISFSLRYAVDNSQARVVTSNEFTVTQRDFVPEFKKVRMQLQVLGFSGTNTGPFPNLDREAGLWGSISLSLNGQDNELVKISQSNPFFFNYREKKETMHPIGFGGIVTVEFDKDPNESLEDFVDHQKMTFVSDLHSTRSIYNYNFGRTTFTHTLGTLYTKYKGDDPIFVLESNNKNVKIHTYVKVLDMKFFN

SEQ ID NO: 35 - protein, 922 aa, Prevotella histicola
MTKFIYAMSLFLLAAISIKAQPIQKTSGCLLHGSVVSSTDATAIAGATVRLYQLKKLVGGTVSDASGNFDVKCPSSGSLQLRITAVGFKEVDTTLNVPTVTPLSIYMRAGKHAMDEVTVTASEKRGMTSTTVIGQTAMEHLQPSSFADLLALLPGGMTKIPALGSANVITLREAGPPSSQYATSSLGTKFVIDGQAIGTDANMQYIAGSFQGDADNSRNHVSYGVDMREIPTDNIEKVEVVRGIPSVKYGELTSGLINITRKRSQSPLLLRLKADEYGKLVSVGKGFLLSGKWNLNVDGGLLDARKEPRNRFETYRRLTFSARLRRKWNLGERYVLEWSGATDYSLNIDNVKTDPEIQIHREDSYRSSYLKMGMNHRLLLRRKALVGLQSVSLAYSASLASDRIHQTEAVALQRDYVVPLAYEGGEYDGLFLPMQYLCDYRVEGKPFYSTLRGETEWLARTSFISHHITAGGEFLLNKNYGRGQIFDITKPLHASTARRPRSYKDIPATDILSFYAEDKATMPIGKHQLTVMAGLRTTQMLNIPASYAVHGKLFTDTRVNVQWDFPSFLGFKSFVSGGLGMMTKMPTVLDLYPDYVYKDITEMNYWDIRPAYKRIHIRTYKLNQVNPDLRPARNKKWEIRLGMDKGAHHFSVTYFHEDMKDGFRSTTTMRPFIYKRYDTSVINPSALTGPPSLASLPVVTDTLLDGYGRTENGSRITKQGIEFQYSSPRIPVIQTRITVNGAWFRTLYENSIPLFRSAPNVVVGTVAIADRYAGYYMSTDKYDKQIFTSNFIFDSYVDKLGLILSATAECFWMSNTKRPATSSTPMGYMDITGTVHPYVEADQSDPYLRWLVLTGTAGQDMDYRERSYMLVNFKATKRFGRHLSLSFFADRVFYVAPDYEVNGFIVRRTFSPYFGMEIGLKI

SEQ ID NO: 36 - protein, 257 aa, Escherichia coli
MLIDFKKVNIYQDERLILKDIDFQATEGEFIYLIGRVGSGKSSLLKTFYGELDIDQEDAEKAEVLGESVLDIKQKRIPALRRQMGIIFQDFQLLHDRSVAKNLKFVLQATGWKDKEKIKQRIKEVLEQVGMIDKAAKMPSELSGGEQQRIAIARAFLNNPKIILADEPTGNLDPETASNIVSILKDTCKNGTTVIMSTHNINLLSQFPGKVYRCMEQALVPVTNEAQTKDLEEDSTSVEPLIEPVLEEEAQAEDSKE

SEQ ID NO: 37 - protein, 508 aa, Lactococcus lactis
MFENQPKALYALALANTGERFGYYTMIAVFALFLRANFGLEPGTAGLIYSIFLGLVYFLPLIGGIMADKFGYGKMVTIGIIVMFAGYLFLSVPLGGGTVAFGAMLAALLLISFGTGLFKGNLQVMVGNLYDTPELASKRDSAFSIFYMAINIGALFAPTAAVKIKEWAETSLGYAGNDAYHFSFAVACVSLIVSMGIYYAFRSTFKHVEGGTKKTEKAAAAAVEELTPQQTKERIVALCLVFAVVIFFWMAFHQNGLTLTYFADEFVSPTSTGVQSMAFDVVNLVMIVFIVYSIMALFQSKTTKAKGIACAVILAAIAVLAYKYMNVNGQVEVSAPIFQQFNPFYVVALTPISMAIFGSLAAKGKEPSAPRKIAYGMIVAGCAYLLMVLASQGLLTPHEQKLAKAAGETVPFASANWLIGTYLVLTFGELLLSPMGISFVSKVAPPKYKGAMMGGWFVATAIGNILVSVGGYLWGDLSLTVVWTVFIVLCLVSASFMFLMMKRLEKVA

SEQ ID NO: 38 - protein, 492 aa, Myroides odoratus
MKKILIFVAGLCMSLAASAQIQRPKLVVGLVVDQMRWDYLYYYYNEYGTDGLRRLVDNGFSFENTHINYAPTVTAIGHSSVYTGSVPAITGIAGNYFFQDDKNVYCCEDPNVKSVGSDSKEGQMSPHRLLASTIGDELQISNDFRSKVIGVALKDRASILPAGHAADAAYWWDTSAGHFVTSTFYTDHLPQWVIDFNEKNHTAPNFNIKTSTQGVTMTFKMAEAALKNENLGKGKETDMLAVSISSTDAIGHVYSTRGKENHDVYMQLDKDLAHFLKTLDEQVGKGNYLLFLTADHGAAHNYNYMKEHRIPAGGWDYRQSVKDLNGYLQGKFGIAPVMAEDDYQFFLNDSLIAASGLKKQQIIDESVEYLKKDPRYLYVFDEERISEVTMPQWIKERMINGYFRGRSGEIGVVTRPQVFGAKDSPTYKGTQHGQPFPYDTHIPFLLYGWNVKHGATTQQTYIVDIAPTVCAMLHIQMPNGCIGTARNMALGN

SEQ ID NO: 39 - protein, 1138 aa, Staphylococcus epidermidis
MDRQVFQTDSRQRWNRFKWTLRVLITIAILLGVVFVAMFALEGSPQMPFRHDYRSVVSASEPLLKDNKRAEVYKSFRDFFKEQKMHSNYAKVAARQHRFVGHTDNVTQKYIKEWTDPRMGIRSAWYVNWDKHAYISLKNNLKNLNMVLPEWYFINPKTDRIEARIDQRALKLMRRAHIPVLPMLTNNYNSAFRPEAIGRIMRDSTKRMGMINELVAACKHNGFAGINLDLEELNINDNALLVTLVKDFARVFHANGLYVTQAVAPFNEDYDMQELAKYDDYLFLMAYDEYNAGSQAGPVSSQRWVEKATDWAAKNVPNDKIVLGMATYGYNWAQGQGGTTMSFDQTMATALNAGAKVNFNDDTYNLNFSYQDEDDGTLHQVFFPDAVTTFNIMRFGATYHLAGFGLWRLGTEDSRIWKYYGKDLSWESAARMPIAKIMQLSGTDDVNFVGSGEVLNVTSEPHAGRIGIVLDKDNQLIIEERYLSLPATYTVQRLGKCKEKQLVLTFDDGPDSRWTPKVLSILKHYKVPAAFFMVGLQIEKNIPIVKDVFNQGCTIGNHTFTHHNMIENSDRRSFAELKLTRMLIESITGQSTILFRAPYNADADPTDHEEIWPMIIASRRNYLFVGESIDPNDWQQGVTADQIYKRVLDGVHQEYGHIILLHDAGGDTREPTVTALPRIIETLQREGYQFISLEKYLGMSRQTLMPPIKKGKEYYAMQANLSLAELIYHISDFLTALFLVFLVLGFMRLVFMYVLMIREKRAENRRNYAPIDPLTAPAVSIIVPAYNEEVNIVRTISNLKEQDYPSLKIYLVDDGSKDNTLQRVREVFENDDKVVIISKKNGGKASALNYGIAACSTDYIVCVDADTQLYKDAVSKLMKHFIADKTGKLGAVAGNVKVGNQRNMLTYWQAIEYTTSQNFDRMAYSNINAITVIPGAIGAFRKDVLEAVGGFTTDTLAEDCDLTMSINEHGYLIENENYAVAMTEAPESLRQFIKQRIRWCFGVMQTFWKHRASLFAPSKGGFGMWAMPNMLIFQYIIPTFSPIADVLMLFGLFSGNASQIFIYYLIFLLVDASVSIMAYIFEHESLWVLLWIIPQRFFYRWIMYYVLFKSYLKAIKGELQTWGVLKRTGHVKGAQTIS

SEQ ID NO: 40 - protein, 508 aa, Propionigenium modestum
MSQINGRISQIIGPVIDVYFDTKGENPEKVLPNIYDALRVKKADGQDLIIEVQQQIGEDTVRCVAMDNTDGLQRGLEVVPTGSPIVMPAGEQIKGRMMNVIGQPIDGMSALQMEGAYPIHREAPKFEDLSTHKEMLQTGIKVIDLLEPYMKGGKIGLFGGAGVGKTVLIMELINNIAKGHNGYSVFAGVGERTREGNDLIRDMLESGVIRYGEKFRKAMDEGKWDLSLVDSEELQKSQATLVYGQMNEPPGARASVALSGLTVAEEFRDHGGKNGEAADIMFFIDNIFRFTQAGSEVSALLGRMPSAVGYQPTLASEMGAMQERITSTKHGSITSVQAVYVPADDLTDPAPATTFTHLDATTELSRKITELGIYPAVDPLGSTSRILDPLIVGKEHYDCAQRVKQLLQKYNELQDIIAILGMDELSDDDKLVVNRARRVQRFLSQPFTVAEQFTGVKGVMVPIEETIKGFNAILNGEVDDLPEQAFLNVGTIEDVKEKAKQLLEATKA

SEQ ID NO: 41 - protein, 984 aa, Prevotella histicola
MNPIYKIITSILFCVLSINTMAQDLTGHVTSKADDKPIAYATVTLKENRLYAFTDEKGNYTIKNVPKGKYTVVFSCMGYASQTVVVMVNAGGATQNVRLAEDNLQLDEVQVVAHRKKDEITTSYTIDRKTLDNQQIMTLSDIAQLLPGGKSVNPSLMNDSKLTLRSGTLERGNASFGTAVEVDGIRLSNNAAMGETAGVSTRSVSASNIESVEVVPGIASVEYGDLTNGVVKVKTRRGSSPFIVEGSINQHTRQIALHKGVDLGGNVGLLNFSIEHARSFLDAASPYTAYQRNVLSLRYMNVFMKKSLPLTLEVGLNGSIGGYNSKADPDRSLDDYNKVKDNNVGGNIHLGWLLNKRWITNVDLTAAFTYADRLSESYTNESSNATQPYIHTLTEGYNIAEDYDRNPSANIILGPTGYWYLRGFNDSKPLNYSLKMKANWSKAFGKFRNRLLVGGEWTSSMNRGRGTYYADMRYAPSWREYRYDALPSLNNIAIYAEDKLSMDVNERQNAELTAGIREDITSIPGSEYGSVGSFSPRMNARYVFRFGQNSWLNSMTLHAGWGRSVKIPSFQVLYPSPSYRDMLAFASTSDADNRSYYAYYTYPSMARYNANLKWQRADQWDLGVEWRTKIADVSLSFFRSKVSNPYMATDVYTPFTYKYTSPAMLQRSGIAVADRRFSIDPQTGIVTVSDASGVKSPVTLGYEERNTYVTNTRYVNADALQRYGLEWIVDFKQIKTLRTQVRLDGKYYHYKAQDETLFADVPVGLNTRQSDGRLYQYVGYYRGGAATTTNYTANASASNGSVSGQVDLNATITTHIPKIRLIVALRLESSLYAFSRATSSRGYVVSSGNEYFGVPYDDKTENQTVIVYPEYYSTWDAPDVLIPFAEKLRWAETNDRGLFNDLAQLVVRTNYPYTLNPNRLSAYWSANLSVTKEIGRHVSVSFYANNFFNTLSQVHSTQTGLETSLFGSGYVPSFYYGLSLRLKI

SEQ ID NO: 42 - protein, 273 aa, Escherichia coli
MERIDISVLMAVYKKDNPAFLRESLESIFSQTVEAAEVVLLEDGPLTDALYDVIKSYEAIYSTLKVVSYPENRGLGKTLNDGLLLCKYNLVARMDADDICKPNRLEMEYNWLKSHEDYDVIGSWVDEFTDNKTRVKSIRKVPEAYDEIKNYAQYRCPINHPTAMYRKAAVLAVGGYLTEYFPEDYFLWLRMLNNGSKFYNIQESLLWFRYSEETVAKRGGWAYACDEVRILVRMLKMGYIPFHVFCQSVVIRFTTRVMPLPIRQRLYNLIRKT

SEQ ID NO: 43 - protein, 507 aa, Paracoccus denitrificans
MSQINGRISQIIGPVIDVYFDTKGENPEKVLPKIHDALRVKRANGQDLIIEVQQHIGEDTVRCVAMDNTDGLQRNLEVVPTGSPIVMPAGDQIKGRMMNVIGQPIDGMEALSMEGAYPIHREAPKFEDLSTHKEMLQTGIKVIDLLEPYMKGGKIGLFGGAGVGKTVLIMELINNIAKGHNGYSVFAGVGERTREGNDLIRDMLESGVIRYGEKFRKAMDEGKWDLSLVDQEELQKSQATLVYGQMNEPPGARASVALSGLTVAEEFRDHGGKNGEAADIMFFIDNIFRFTQAGSEVSALLGRMPSAVGYQPTLASEMGTMQERITSTKHGSITSVQAVYVPADDLTDPAPATTFTHLDATTELSRKITELGIYPAVDPLGSTSRILDPLIVGKDHYECAQRVKQLLQHYNELQDIIAILGMDELSDEDKLVVNRARRVQRFLSQPFTVAEQFTGVKGVMVPIEETIKGFNAILNGEVDDLPEQAFLNVGTIEDVKEKAKRLLEATK

SEQ ID NO: 44 - protein, 268 aa, Mycobacterium tuberculosis
MPIGNGQKYQLTIINHTEIIMLIDYKKVNIYQDERLILKDVDFQAETGEFIYLIGRVGSGKSSLLKTIYGELDIDSEDAEKAVVLDESMPNIKRSRIPALRKQMGIIFQDFQLLHDRSVAKNLKFVLQATGWTSKQKIERRIEEVLAQVGMTDKKNKMPSELSGGEQQRIAIARALLNTPKIIIADEPTGNLDPETAANIVSILKDSCQAGTTVIMSTHNINLIDQFPGKVYRCHEGELHQLTDKKEVSELAEETAPVETIDEPEQND

SEQ ID NO: 45 - protein, 336 aa, Yersinia pestis
MKRNILLFICLATSILLLFGLNLTTGSVQIPFADILDILCGRFIGKESWEYIILENRLPQTLTAILCGASLSVCGLMLQTAFRNPLAGPDVFGISSGAGLGVALVMLLLGGTVSTSIFTVSGFLAILTAAFVGAIAVTALILFLSTLVRNSVLLLIVGIMVGYVSSSAVSLLNFFASEEGVKSYMVWGMGNFGAVSMNHIPLFSILCLIGIIASFLLVKPLNILLLGPQYAESLGISTRQIRNILLVVVGLLTAITTAFCGPISFIGLAIPHIARLLFRTENHQILLPGIVLSGAAIALLCNFICYLPGESGIIPLNAVTPLIGAPIIIYVIIQRR

SEQ ID NO: 46 - protein, 408 aa, Bacillus subtilis
MKKYYPWVLVALLWFVALLNYMDRQMLSTMQEAMKVDIAELNHAEAFGALMAVFLWIYGIVSPFAGIIADRVNRKWLVVGSIFVWSAVTYLMGYAESFDQLYWLRAFMGISEALYIPAALSLIADWHEGKSRSLAIGIHMTGLYVGQAVGGFGATLAAMFSWHAAFHWFGIIGIVYSLVLLLFLKENPKHGQKSVLQGETKPSKNPFRGLSIVFSTWAFWVILFYFAVPSLPGWATKNWLPTLFANSLDIPMSSAGPMSTITIAVSSFIGVIMGGVISDRWVQRNLRGRVYTSAIGLGLTVPALMLLGFGHSLVSVVGAGLCFGIGYGMFDANNMPILCQFISSKYRSTAYGIMNMTGVFAGAAVTQVLGKWTDGGNLGNGFAILGGIVVLALVLQLSCLKPTTDNME

SEQ ID NO: 47 - protein, 694 aa, Mycobacterium tuberculosis
MVTKKTTTKKAPVKKTSAKTTKVKEPSHIGLVKNDAYLAPYEDAIRGRHEHALWKMNQLTQNGKLTLSDFANGHNYYGLHQTADGWVFREWAPNATEIYLVGDFNGWNEQEAYQCHRIEGTGNWELTLPHDAMQHGQYYKMRVHWEGGEGERIPAWTQRVVQDEASKIFSAQVWAPAEPYVWEKKTFKPQTSPLLIYECHIGMAQDEEKVGTYNEFREKVLPRIIKDGYNAIQIMAIQEHPYYGSFGYHVSSFFAASSRFGTPEELKALIDEAHKNGIAVIMDIVHSHAVKNEVEGLGNLAGDPNQYFYPGERHEHPAWDSLCFDYGKDEVLHFLLSNCKYWLEEYHFDGFRFDGVTSMLYYSHGLGEAFCNYADYFNGHQDDNAICYLTLANCLIHEVNKNAVTIAEEVSGMPGLAAKFKDGGYGFDYRMAMNIPDYWIKTIKELPDEAWKPSSIFWEIKNRRSDEKTISYCESHDQALVGDKTIIFRLVDADMYWHFRKGDETEMTHRGIALHKMIRLATIAAINGGYLNFMGNEFGHPEWIDFPREGNGWSHKYARRQWNLVDNEELCYHLLGDFDRKMLEVITSEKKFNETPIQEIWHNDGDQILAFSRGELVFVFNFSPSHSYSDYGFLVPEGSYNVVLNTDAREFGGFGFADDTVEHFTNSDPLYEKDHKGWLKLYIPARSAVVLRKK

SEQ ID NO: 48 - protein, 448 aa, Prevotella melaninogenica
MKIDIERIKYFLTVGMFMKTEHSSKRRNMLIRQFQKFYLTVKFFFVRDHAASTAQLSFSTIMAIVPIASMIFAIANGFGFGQFLEKQFREMLSAQPEAATWLLKLTQSYLVHAKTGLFIGIGLMIMLYSVFSLIRTVETTFDNIWQVKDSRPISRIVIDYTALMFLVPISIIILSGLSIYFYSFVENLNGLRFLGTIASFSLRYLVPWAILTLMFIVLYVFMPNAKVKITKTVAPAMIASIAMLCLQAVYIHGQIFLTSYNAIYGSFAALPLFMLWILASWYICLFCAELCYFNQNLEYYECLIDTEDICHNDLLILCATVLSHICQRFANDQKPQTALQIKTETHIPIRVMTDILYRLKEVNLISENFSPTSDEVTYTPTHDTNNITVGEMIARLESTPASDFALLGFSPKKAWNHDIYDRVGSIREIYLNELKSINIKELISYSEN

SEQ ID NO: 49 - protein, 326 aa, Bacillus anthracis
MMKRPSIARVVKVIICLLTPILLSFSGIGDNDIDKKKSTSKEVDDTLRIVITGDLLLDRGVRQKIDMAGVDALFSPTIDSLFHSSNYVIANLECPVTKIRERVFKRFIFRGEPEWLPTLRRHGITHLNLANNHSIDQGRNGLLDTQEQIKKAGMIPIGAGKNMEEAAEPVLISTSPRHVWVISSLRLPLENFLYLPQKPCVSQESIDSLIMRVKRLRATDKNCYILLILHWGWEHHFRATPQQREDAHKLIDAGADAIVGHHSHTLQTIETYRGKPIYYGIGNFIFDQRKPMNSRACLVELSITAEKCKAKALPIEIKNCTPYLSK

SEQ ID NO: 50 - protein, 259 aa, Helicobacter pylori
MILLSFDTEEFDVPREHGVDFSLEEGMKVSIEGTNRILDILKANNVCATFFCTGNFAELAPEVMERIKNEGHEVACHGVDHWQPKPEDVFRSKEIIERVTGVKVAGYRQPRMFPVSDEDIEKAGYLYNSSLNPAFIPGRYMHLTTSRTWFMQGKVMQIPASVSPHLRIPLFWLSMHNFPEWFYLRLVRQVLRHDGYFVTYFHPWEFYDLKSHPEFKMPFIIKNHSGHELEQRLDRFIKAMKADKQEFITYVDFVNRQKK

SEQ ID NO: 51 - protein, 252 aa, Escherichia coli
MAKNISFTIKYWKQNGPQDQGHFDTHEMKNIPDDTSFLEMLDILNEELIAAGDEPFVFDHDCREGICGMCSLYINGTPHGKTERGATTCQLYMRRFNDGDVITVEPWRSAGFPVIKDCMVDRTAFDKIIQAGGYTTIRTGQAQDANAILISKDNADEAMDCATCIGCGACVAACKNGSAMLFVSSKVSQLALLPQGKPEAAKRAKAMVAKMDEVGFGNCTNTRACEAVCPKNEKIANIARLNREFIKAKFAD

SEQ ID NO: 52 - protein, 398 aa, Mycobacterium tuberculosis
MSENKLSTNEQAQTADAPVKASYTEYKVIPSQGYCMIVKCRKGDQTVVLKTLKEEYRERVLLRNALKREFKQCQRLNHSGIVRYQGLVEVDGYGLCIEEEYVEGRTLQAYLKENHTDDEKIAIINQIADALRYAHQQGVIHRNLKPSNVLVTTQGDYVKLIDFSVLSPEDVKPTAETTRFMAPEMKDETLTADATADIYSLGTIMKVMGLTLAYSEVIKRCCAFKRSDRYSNVDELLADLNNEGSSFSMPKIGKGTVVLGLIIAVVIGIGALLYNYGGALIDQVGKIDVSSVFSSDAETAPEDTVKVNTAEQSDSLSTEAEAPAIGKLAFMNRMKPALYKDLDNIFEKNSADKAKLTKAIKTYYRGLIQANDTLDNEQRAEVDRVFGDYVKQKKAALN

SEQ ID NO: 53 - protein, 522 aa, Bacillus subtilis
MRKYICLLLFYLFTFLPLSAQQGNDSPLRKLQLAEMAIKNFYVDSVNEQKLVEDGIRGMLEKLDPHSTYTDAKETKAMNEPLQGDFEGIGVQFNMIEDTLVVIQPVVNGPSQKVGILAGDRIVSVNDSTIAGVKMARIDIMKMLRGKKGTKVKLGVVRRGVKGVLTFVVTRAKIPVHTINASYMIRPNVGYIRIESFGMKTHDEFMSAVDSLKKKGMKTLLLDLQDNGGGYLQSAVQISNEFLKNNDMIVYTEGRRARRQNFKAIGNGRLQDVKVYVLVNELSASAAEIVTGAIQDNDRGTVVGRRTFGKGLVQRPFDLPDGSMIRLTIAHYYTPSGRCIQKPYTKGDLKDYEMDIEKRFKHGELTNPDSIQFSDSLKYYTIRKHRVVYGGGGIMPDNFVPLDTTKFTRYHRMLAAKSIIINAYLKYADANRQALKAQYSSFDAFNKGYVVPQSLLDEIVAEGKKEKIEPKDAAELKATLPNIALQIKALTARDIWDMNEYFRVWNTQSDIVNKAVALATGK

SEQ ID NO: 54 - protein, 348 aa, Prevotella melaninogenica
MKLTEQRSSMLHGVLLITLFACAAFYIGDMGWVKALSLSPMVVGIILGMLYANSLRNNLPDTWVPGIAFCGKRVLRFGIILYGFRLTFQDVVAVGFPAIIVDAIIVSGTILLGVLVGRLLKMDRSIALLTACGSGICGAAAVLGVDGAIRPKPYKTAVAVATVVIFGTLSMFLYPILYRAGIFDLSPDAMGIFAGSTIHEVAHVVGAGNAMGAAVSNSAIIVKMIRVMMLVPVLLVIAFFVAKNVAERDDEAGGSRKINIPWFAILFLVVIGFNSLNLLPKELVDFINTLDTFLLTMAMSALGAETSIDKFKKAGFKPFLLAAILWCWLIGGGYCLAKYLVPVLGVAC

SEQ ID NO: 55 - protein, 833 aa, Prevotella sp.
MNKQFLLAALWLSPLGLYAHKANGIGAVTWKNEAPKERMIRGIDEDKTHQRFTLSGYVKDRNGEPLINATIYDLTTRQGTMTNAYGHFSLTLGEGQHEIRCSYVGYKTLIETIDLSANQNHDIILQNEAQLDEVVVTTDLNSPLLKTQTGKLSLSQKDIKTEYALLSSPDVIKTLQRTSGVADGMELASGLYVHGGNGDENLFLLDGTPLYHTNHSLGLFSSFNADVVKNVDFYKSGFPARYGGRLSSVIDVRTADGDLYKTHGSYRIGLLDGAFHIGGPIRKGKTSYNFGLRRSWMDLLTRPAFAIMNHKSDNEDKLSMSYFFHDLNFKLTNIFNERSRMSLSVYSGEDRLDAKDEWHSNNSSGYNDVDIYVNRFHWGNFNAALDWNYQFSPKLFANFTAVYTHNRSTVSSSDEWRFTRPGEKEQLTLTSHGYRSSIDDIGYRAAFDFRPSPRHHIRFGQDYTYHRFQPQTYNRFDNYQTNSEAKADTIATHSYNKNVAHQLTFYAEDEMTLNEKWSLNGGVNADVFHISGKTFATLSPRLSMKFQPTERLSLKASYTLMSQFVHKIANSFLDLPTDYWVPTTARLHPMRSWQVAAGAYMKPNKHWLLSLEAYYKRSSHILQYSSWAGLEPPAANWDYMVMEGDGRSYGVELDADYNVSNLTLHGSYTLSWTQKKFDDFYDGWYYDKFDNRHKLTLTGRWNITKKIAAFAAWTFRTGNRMTIPTQYIGLPDVPAQEQGGLTFNSSDDNTLNFAYEKPNNVILPAYHRLDIGFDFHHTTKKGHERIWNLSFYNAYCHLNSLWVRVKIDSNNQMKIRNIAFIPVIPSFSYTFKF

SEQ ID NO: 56 - protein, 1133 aa, Escherichia coli
MSKQVFQTDSRQRWSYFKWTLRVILTILSLLGIVFLAMFALEGSPQMPFRHDYRNAVTAASPYTKDNKTAKLYKSFRDFFKEKKMHNNYAKATIKKQRFIGKADSVTQKYFREWDDPRIGVRSAWYVNWDKHAYISLKNNIKHLNMVLPEWFFINPKTDKVEYRIDKQALRLMRRTGIPVLPMLTNNYNSDFHPEAIGRIMRDEKKRMALINEMVRTCRHYGFAGINLDLEELNIQDNDLLVELLKDFSRVFHANGLYVTQAVAPFNEDYNMQELAKYNDYLFLMAYDEHNIESQPGAVSSQRWVEKATDWAAKNVPNDKIVLGMATYGYDWANGEGGTTVSFDQTMAIAQDADAKVKFDDDTYNVNFSYQNTDDGKIHHVFFTDAATTFNIMRFGAEYHLAGYGLWRLGTEDKRIWRFYGKDMSWENVARMSVAKLMQLNGTDDVNFVGSGEVLEVTTEPHPGDISIRIDKDNRLISEEYYRALPSTYTIQRLGKCKDKQLVITFDDGPDSRWTPTVLSTLKKYNVPAAFFMVGLQMEKNLPLVKQVYEDGHTIGNHTFTHHNMIENSDRRSYAELKLTRMLIESVTGHSTILFRAPYNADADPTEHEEIWPMIVASRRNYLFVGESIDPNDWEPNVTSDQIYQRVIDGVHHEDGHIILLHDAGGSSRKPTLDALPRIIETLQHEGYQFISLEQYLGMGKQTLMPEINKGKAYYAMQTNLWLAEMIYHVSDFLTALFLVFLALGMMRLIFMYVLMIREKRAENRRNYAPIDAATAPAVSIIVPGYNEEVNIVRTITTLKQQDYPNLHIYFVDDGSKDHTLERVHEAFDNDDTVTILAKKNGGKASALNYGIAACRSEYVVCIDADTQLKNDAVSRLMKHFIADTEKRVGAVAGNVKVGNQRNMLTYWQAIEYTSSQNFDRMAYSNINAITVVPGAIGAFRKEVIEAVGGFTTDTLAEDCDLTMSINEHGYIIENENYAVALTEAPETLRQFVKQRIRWCFGVMQAFWKHRSSLFAPSKKGFGLWAMPNMLIFQYIIPTFSPLADVLMLIGLFTGNALQIFFYYLIFLVIDASVSIMAYIFEGERLWVLLWVIPQRFFYRWIMYYVLFKSYLKAIKGELQTWGVLKRTGHVKG

SEQ ID NO: 57 - protein, 292 aa, Bacillus subtilis
MAKKRNKARSRHSLQVVTLCISTAMVLMLIGIVVLTGFTSRNLSSYVKENLTITMILQPDMNTEESAALCERIRTLHYINSLNFISKEQALKDGTKELGANPAEFAGENPFTGEIEVQLKANYANNDSIRNIVQQLRTYRGVSDITYPQSLVESVNQTLGKISLVLLVIAVLLTIISFSLINNTIRLSIYAHRFSIHTMKLVGGSWSFIRAPFLRRAVLEGLVSALLAIAVLGIGICLLYEKEPEITKLLSWDALIITAIVMLAFGVIIATFCAWLSVNKFLRMKAGDLYKI

SEQ ID NO: 58 - protein, 254 aa, Haemophilus influenzae
MKNIYFLSDAHLGSLAIDHRRTHERRLVRFLDSIKHKAAAVYLLGDMFDFWNEYKYVVPKGFTRFLGKISELTDMGVEVHFFTGNHDLWTYGYLEKECGVILHRKPITTEIYDKVFYLAHGDGLGDPDPMFRFLRKVFHNRFCQRLLNFFHPWWGMQLGLNWAKRSRLKRKDGKEVPYLGEDKEYLVQYTKEYMSTHKDIDYYIYGHRHIELDLTLSRKARLLILGDWIWQFTYAVFDGEHMFLEEYVEGESKP

SEQ ID NO: 59 - protein, 388 aa, Escherichia coli
MVGLDVLCYFIHAKGREKECYFERIIYQITCHSRTKCYLCNIMKYSIIVPVFNRPDEVEELLESLLSQEEKDFEVVIVEDGSQIPCKEVCDKYADKLDLHYYSKENSGPGQSRNYGAERAKGEYLLILDSDVVLPKGYICAVSEELKREPADAFGGPDCAHESFTDTQKAISYSMTSFFTTGGIRGGKKKLDKFYPRSFNMGIRRDVYQELGGFSKMRFGEDIDFSIRIFKAGKRCRLFPEAWVWHKRRTDFRKFWKQVYNSGIARINLYKKYPESLKLVHLLPMVFTVGTALLVLMILFGLFLQLFPIINVFGSVFIMMGLMPLVLYSVIICVDSTMQNNSLNIGLLSIEAAFIQLTGYGCGFISAWWKRCVCGMDEFAAYEKNFYK

SEQ ID NO: 60 - protein, 435 aa, Streptococcus mutans
MKIEKVHAREIMDSRGNPTVEVEVTLENGVMGRASVPSGASTGENEALELRDGDKNRFLGKGVLKAVENVNNLIAPALKGDCVLNQRAIDYKMLELDGTPTKSKLGANAILGVSLAVAQAAAKALNIPLYRYIGGANTYVLPVPMMNIINGGAHSDAPIAFQEFMIRPVGAPSEKEGIRMGAEVFHALAKLLKKRGLSTAVGDEGGFAPKFDGIEDALDSIIQAIKDAGYEPGKDVKIAMDCAASEFAVCEDGKWFYDYRQLKNGMPKDPNGKKLSADEQIAYLEHLITKYPIDSIEDGLDENDWENWVKLTSAIGDRCQLVGDDLFVTNVKFLEKGIKMGAANSILIKVNQIGSLTETLEAIEMAHRHGYTTVTSHRSGETEDTTIADIAVATNSGQIKTGSMSRTDRMAKYNQLIRIEEELGACAKYGYAKLK

SEQ ID NO: 61 - protein, 501 aa, Brucella suis
MKKLFTIAMLLGVTLGIHAQEVYSLQKCRELALQNNRQLKVSRMTVDVAENTRKAAKTKYLPRVDALAGYQHFSREISLLSDDQKNAFSNLGTNTFGQLGGQIGQNLTSLAQQGILSPQMAQQLGQLFSNVATPLTQVGNNIGQSINDAFRSNTKNVYAGGIVVNQPIYMGGAIKAANDMAAIGEQVAQNNISLKRQLVLYGVDNAYWLAISLKKKEALAIRYRDLAQKLNEDVKKMIREGVATRADGLKVEVAVNTADMQIARIQSGVSLAKMALCELCGLELNGDIPLSDEGDADLPPTPSTQFDNYTVSSSDTTGLNEARPELRLLQNAVDLSIQNTKLIRSLYMPHVLLTAGYSVSNPNLFNGFQKRFTDLWNIGITVQVPVWNWGENKYKVRASKTATTIAQLEMDDVRKKIDLEIEQNRLRLKDANKQLATSQKNMAAAEENLRCANVGFKEGVMTVTEVMAAQTAWQTSRMAIIDAEISVKLAQTGLQKALGGL

SEQ ID NO: 62 - protein, 577 aa, Salmonella enterica
MKRTFVTKMVKPIEENSLFFMFMLLVGAFTNVSHRNVFGYIELIADVYIICFLLSLCQRTIRQGLVIMLSSVIYVVAIIDTCCKTLFDTPITPTMLLLAQETTGREATEFFLQYLNLKLFFSAADIILFLAFCHIVMAVKKMKFSTSYLKQPFVAFVLMFTIFVGMALSIYDKVQLYTVKNLSGLEVAVTNGFAHLYHPVERIVYGLYSNHLIAKQVDGVIMANQQIKVDSCSFTSPTIVLVIGESANRHHSQLYGYPLPTTPYQLAMKNGKDSLAVFTNVVSPWNLTSKVFKQIFSLQSVDEKGDWSKYVLFPAVFKKAGYHVSFLSNQFPYGINYTPDWTNNLVGGFFLNHPQLNKQMFDYRNVTIHNYDEDLLNDYKEIISYKKPQLIIFHLLGQHFQYSLRCKSNMKKFGIKDYKRMDLTDKEKQTIADYDNATLYNDFVLNKIVEQFRNKDAIIVYLSDHGEDCYGKDVNMAGRLTEVEQINLKKYHEEFEIPFWIWCSPIYKQRHRKIFTETLMARNNKFMTDDLPHLLLYLAGIKTKDYCEERNVISPSFNNNRRRLVLKTIDYDKALYQ

SEQ ID NO: 63 - protein, 517 aa, Escherichia coli
MFKNHPKGLLQAAFSNMGERFGYYIMNAVLALFLCSKFGLSDETSGLIASLFLAAIYVMSLVGGVIADRTQNYQRTIESGLVVMALGYVALSIPVLATPENNSYLLAFTIFALVLIAVGNGLFKGNLQAIVGQMYDDFETEAAKVSPERLKWAQGQRDAGFQIFYVFINLGALAAPFIAPVLRSWWLGRNGLTYDAALPQLCHKYINGTIGDNLGNLQELATKVGGNSADLASFCPHYLDVFNTGVHYSFIASVVTMLISLIIFMSSKKLFPMPGKKEQIVNVEYTDEEKASMAKEIKQRMYALFAVLGISVFFWFSFHQNGQSLSFFARDFVNTDSVAPEIWQAVNPFFVISLTPLIMWVFAYFTKKGKPISTPRKIAYGMGIAGFAYLFLMGFSLVHNYPSAEQFTSLEPAVRATMKAGPMILILTYFFLTVAELFISPLGLSFVSKVAPKNLQGLCQGLWLGATAVGNGFLWIGPLMYNKWSIWTCWLVFAIVCFISMVVMFGMVKWLERVTKS

SEQ ID NO: 64 - protein, 395 aa, Pseudomonas aeruginosa
MQKKIKIGLLPRVIIAILLGLFLGYYLPDPAVRVFLTFNSIFSQFLGFMIPLIIIGLVTPAIAGIGKGAGKLLLATVAIAYVDTIVAGGLSYGTGTWLFPSMIASTGGAIPHIDKATELTPYFTINIPAMVDVMSSLVFSFIAGLGIAYGGLRTMENLFNEFKTVIEKVIEKAIIPLLPLYIFGVFLSMTHNGQARQVLLVFSQIIIVILVLHVLILIYEFCIAGAIVKHNPFRLLWNMLPAYLTALGTSSSAATIPVTLKQTVKNGVSEEVAGFVVPLCATIHLSGSAMKITACALTICMLTDLPHDPGLFIYFILMLAIIMVAAPGVPGGAIMAALAPLSSILGFNEEAQALMIALYIAMDSFGTACNVTGDGAIALAVNKFFGKKKETSILS

SEQ ID NO: 65 - protein, 208 aa, Escherichia coli
MISVYSIKPQFQRVLTPILELLHRAKVTANQITLWACVLSLVIGILFWFAGDVGTWLYLCLPVGLLIRMALNALDGMMARRYNQITRKGELLNEVGDVVSDTIIYFPLLKYHPESLYFIVAFIALSIINEYAGVMGKVLSAERRYDGPMGKSDRAFVLGLYGVVCLFGINLSGYSVYIFGVIDLLLVLSTWIRIKKTLKVTRNSQTPE

SEQ ID NO: 66 - protein, 582 aa, Escherichia coli
MKLSTILLSIMLGLSSSTMAQQKDVTIKLIETTDVHGSFFPYDFITRKPKSGSMARVYTLVEELRKKDGKDNVYLLDNGDILQGQPISYYYNYVAPEKTNIAASVLNYMGYDVATVGNHDIETGHKVYDKWFKELKFPILGANIIDTKTNKPYILPYYTIKKKNGIKVCVIGMLTPAIPNWLKESIWSGLRFEEMVSCAKRTMAEVKTQEKPDVIVGLFHSGWDGGIKTPEYDEDASKKVAKEVPGFDIVFFGHDHTPHSSIEKNIVGKDVICLDPANNAQRVAIATLTLRPKTVKGKRQYTVTKATGELVDVKELKADDAFIQHFQPEIDAVKAWSDQVIGRFENTIYSKDSYFGNSAFNDLILNLELEITKADIAFNAPLLFNASIKAGPITVADMFNLYKYENNLCTMRLTGKEIRKHLEMSYDLWCNTMKSPEDHLLLLSSTQNDAQRLGFKNFSFNFDSAAGIDYEVDVTKPDGQKVRILRMSNGEPFDENKWYTVAVNSYRANGGGELLTKGAGIPRDSLKSRIIWESPKDQRHYLMEEIKKAGVMNPQPNHNWKFIPETWTVPAAARDRKLLFGE

SEQ ID NO: 67 - protein, 826 aa, Mycobacterium tuberculosis
MKLSELKTGETGVIVKVSGHGGFRKRIIEMGFIKGKTVEVLLNAPLQDPVKYKIMGYEVSLRHSEADQIEVLSDVKTHSVGNEEEQEDNQLEMDSTTYDSTDKELTPEKQSDAVRRKNHTINVALVGNPNCGKTSLFNFASGAHERVGNYSGVTVDAKVGRAEFDGYVFNLVDLPGTYSLSAYSPEELYVRKQLVDKTPDVVINVIDSSNLERNLYLTTQLIDMHIRMVCALNMFDETEQRGDHIDAQKLSELFGVPMIPTVFTNGRGVKELFRQIIAVYEGKEDESLQFRHIHINHGHEIENGIKEMQEHLKKYPELCHRYSTRYLAIKLLEHDKDVEQLVSPLGDSIEIFNHRDTAAARVKEETGNDSETAIMDAKYGFINGALKEANFSTGDKKDTYQTTHVIDHVLTNKYFGFPIFFLVLLVMFTATFVIGQYPMDWIEAGVGWLGEFISKNMPAGPVKDMIVDGIIGGVGAVIVFLPQILILYFFISYMEDCGYMSRAAFIMDRLMHKMGLHGKSFIPLIMGFGCNVPAVMATRTIESRRSRLITMLILPLMSCSARLPIYVMITGSFFALKYRSLAMLSLYIIGVLMAVAMSRLFSAFVVKGEDTPFVMELPPYRFPTWKAIGRHTWEKGKQYLKKMGGIILVASIIVWALGYFPLPDDPNMDNQARQEQSYIGRIGKAVEPVFRPQGFNWKLDVGLLSGMGAKEIVASTMGVLYSNDGSFSDDNGYSSETGKYSKLHNLITKDVATMHHISYEEAEPIATLTAFSFLLFVLLYFPCVATIAAIKGETGSWGWALFAAGYTTALAWIVSAVVFQVGMLFM

SEQ ID NO: 68 - protein, 437 aa, Mycobacterium tuberculosis
MESFIIEGGHQLSGTIAPQGAKNEALEVICATLLTSEEVIIRNVPDILDVNNLIKLLQDIGVKVKKLAPNEFSFQADEVNLDYLESSDFVKKCSSLRGSVLMIGPLLGRFGKATIAKPGGDKIGRRRLDTHFLGFKNLGAHFGRVEDRDVYEIQADKLVGTYMLLDEASITGTANIIMAAVLAEGTTTIYNAACEPYIQQLCKMLNAMGAKISGIASNLITIEGVKELHSADHRILPDMIEVGSFIGIAAMIGDGVRIKDVSVPNLGLILDTFHRLGVQIIVDNDDLIIPRQDHYVIDSFIDGTIMTISDAPWPGLTPDLISVLLVVATQAQGSVLFHQKMFESRLFFVDKLIDMGAQIILCDPHRAVVVGHDNAKKLRAGRMSSPDIRAGIALLIAALTAQGTSRIDNIVQIDRGYENIEGRLNALGAKIQRAEVC

SEQ ID NO: 69 - protein, 244 aa, Bacillus spizizenii
MNIAVIFAGGSGLRMHTKSRPKQFLDLNGKPIIIYTLELFDNHPNIDAIVVACIESWIPFLEKQLRKFEINKVVKIIPGGKSGQESIYKGLCAAEEYAQSKGVSNEETTVLIHDGVRPLITEETITDNIKKVEEVGSCITCIPATETLIVKQADDALEIPSRADSFIARAPQSFRLIDIITAHRRSLAEGKADFIDSCTMMSHYGYKLGTIIGPMENIKITTPTDFFVLRAMVKVHEDQQIFGL

(19) United States
(12) Patent Application Publication
(10) Pub. No.: US 2025/0127823 A1
(43) Pub. Date: Apr. 24, 2025

(54) COMPOSITIONS AND METHODS OF AFFECTING CYTOKINE LEVELS USING PREVOTELLA HISTICOLA

(71) Applicant: Evelo Biosciences, Inc., Cambridge, MA (US)

(72) Inventors: Andrea Itano, Arlington, MA (US); Douglas Maslin, Cambridge (GB); Duncan McHale, Kent (GB)

(21) Appl. No.: 18/836,109

(22) PCT Filed: Feb. 7, 2023
(86) PCT No.: PCT/US2023/012489

§ 371 Date: Aug. 6, 2024

Related U.S. Application Data
(60) Provisional application No. 63/329,174, filed on Apr. 8, 2022.
(60) Provisional application No. 63/314,019, filed on Feb. 25, 2022.
(60) Provisional application No. 63/307,365, filed on Feb. 7, 2022.

(51) Int. Cl. A61K 35/74; A61K 9/28; A61P 29/00

(52) U.S. Cl. CPC A61K 35/74; A61K 9/2833; A61P 29/00

(57) ABSTRACT

Provided herein are methods and compositions related to Prevotella histicola bacteria for the reduction of IL-23, LL-12b, and IL-17 levels in a subject.

### CROSS-REFERENCE TO RELATED APPLICATIONS

This application claims the benefit of U.S. Provisional Application No. 63/307,365, filed on Feb. 7, 2022, U.S. Provisional Application No. 63/314,019, filed on Feb. 25, 2022, and U.S. Provisional Application No. 63/329,174, filed on Apr. 8, 2022, the content of each of which is hereby incorporated by reference in its entirety.

### SUMMARY

Provided herein are methods and compositions related to the use of certain strains of Prevotella histicola for the reduction of levels (e.g., protein or RNA levels) of one or more cytokines (e.g., IL-23, IL-12b, and/or IL-17 levels) in a subject in need thereof. In some embodiments, the method decreases RNA levels. In some embodiments, the Prevotella histicola strain is a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a bacterial composition (such as a pharmaceutical composition) and/or a solid dosage form. In some embodiments, the Prevotella histicola strain is Prevotella histicola Strain B (NRRL accession number B 50329). In certain aspects, provided herein is a method of reducing IL-23 levels (e.g., protein or RNA levels) in a subject. In some embodiments, the method decreases IL-23 RNA levels. In some embodiments, the method decreases IL-23 levels (e.g., protein or RNA levels) in the skin of the subject, e.g., in a skin lesion of the subject. For example, the lesion can be associated with psoriasis or atopic dermatitis. In certain aspects, provided herein is a method of reducing IL-12b levels (e.g., protein or RNA levels) in a subject. In some embodiments, the method decreases IL-12b RNA levels. In some embodiments, the method decreases IL-12b levels (e.g., protein or RNA levels) in the skin of the subject, e.g., in a skin lesion of the subject. For example, the lesion can be associated with psoriasis or atopic dermatitis. In certain aspects, provided herein is a method of reducing IL-17 levels (e.g, protein or RNA levels) in a subject. In some embodiments, the methods decrease IL-17 RNA levels. In some embodiments, the methods decrease IL-17 levels (e.g., protein or RNA levels) in the skin of the subject, e.g., in a skin lesion of the subject. For example, the lesion can be associated with psoriasis or atopic dermatitis.

In certain aspects, provided herein is a method of reducing levels (protein or RNA levels) of one or more cytokines (e.g., IL-23, IL-12b, and/or IL-17 levels) in a subject in need thereof, comprising administering to the subject a Prevotella histicola strain. In certain aspects, provided herein is a dose (e.g., a therapeutically effective dose) of a Prevotella histicola strain for use in reducing levels (protein or RNA levels) of one or more cytokines (e.g., IL-23, IL-12b, and/or IL-17 levels) in a subject in need thereof. In certain aspects, provided herein is use of a dose (e.g., a therapeutically effective dose) of a Prevotella histicola strain for the preparation of a medicament for reducing levels (protein or RNA levels) of one or more cytokines (e.g., IL-23, IL-12b, and/or IL-17 levels) in a subject in need thereof. In some embodiments, the Prevotella histicola strain is a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a bacterial composition (such as a pharmaceutical composition) and/or a solid dosage form. In some embodiments, the Prevotella histicola strain is Prevotella histicola Strain B (NRRL accession number B 50329). In certain aspects, provided herein is a method of reducing IL-23 levels (e.g., protein or RNA levels). In some embodiments, the method decreases IL-23 RNA levels. In some embodiments, the method decreases IL-23 levels (e.g., protein or RNA levels) in the skin of the subject, e.g., in a skin lesion of the subject. For example, the lesion can be associated with psoriasis or atopic dermatitis. In certain aspects, provided herein is a method of reducing IL-12b levels (e.g., protein or RNA levels). In some embodiments, the method decreases IL-12b RNA levels. In some embodiments, the method decreases IL-12b levels (e.g., protein or RNA levels) in the skin of the subject, e.g., in a skin lesion of the subject. For example, the lesion can be associated with psoriasis or atopic dermatitis. In certain aspects, provided herein is a method of reducing IL-17 levels (e.g., protein or RNA levels). In some embodiments, the method decreases IL-17 RNA levels. In some embodiments, the methods decrease IL-17 levels (e.g., protein or RNA levels) in the skin of the subject, e.g., in a skin lesion of the subject. For example, the lesion can be associated with psoriasis or atopic dermatitis.

Provided herein are methods and compositions related to the use of certain strains of Prevotella histicola for the reduction (e.g., statistically significant reduction) of levels (e.g., protein or RNA levels) of one or more cytokines (e.g., IL-1b, IL-2, IFNg (also referred to as IFN-gamma), IL-4, IL-5, IL-6, IL-8, IL-10, TNFa (also referred to as TNF-alpha or TNF) and/or IL-17 levels levels) in a subject in need thereof. In some embodiments, the Prevotella histicola strain is a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a bacterial composition (such as a pharmaceutical composition) and/or a solid dosage form. In some embodiments, the Prevotella histicola strain is Prevotella histicola Strain B (NRRL accession number B 50329). In certain aspects, provided herein is a method of reducing IL-1b, IL-2, IFNg, IL-4, IL-5, IL-6, IL-8, IL-10, TNFa and/or IL-17 levels (e.g., protein or RNA levels) in a subject. In some embodiments, the method decreases IL-1b, IL-2, IFNg, IL-4, IL-5, IL-6, IL-8, IL-10, TNFa and/or IL-17 levels (e.g., protein or RNA levels) in blood of the subject. In some embodiments, the method decreases IL-1b, IL-2, IFNg, IL-4, IL-5, IL-6, IL-8, IL-10, TNFa and/or IL-17 levels (e.g., protein or RNA levels) in peripheral blood mononuclear cells (PBMCs) of the subject. In certain aspects, provided herein is a method of reducing IL-6, IL-8, and/or TNFa levels (e.g., protein or RNA levels) in a subject. In some embodiments, the method decreases IL-6, IL-8, and/or TNFa levels (e.g., protein or RNA levels) in blood of the subject. In some embodiments, the method decreases IL-6, IL-8, and/or TNFa levels (e.g., protein or RNA levels) in peripheral blood mononuclear cells (PBMCs) of the subject. In certain aspects, provided herein is a method of reducing IL-6 levels (e.g., protein or RNA levels) in a subject. In some embodiments, the method decreases IL-6 levels (e.g., protein or RNA levels) in blood of the subject. In some embodiments, the method decreases IL-6 levels (e.g., protein or RNA levels) in peripheral blood mononuclear cells (PBMCs) of the subject. In certain aspects, provided herein is a method of reducing IL-8 levels (protein or RNA levels) in a subject. In some embodiments, the method decreases IL-8 levels (e.g., protein or RNA levels) in blood of the subject. In some embodiments, the method decreases IL-8 levels (e.g., protein or RNA levels) in peripheral blood mononuclear cells (PBMCs) of the subject. In certain aspects, provided herein is a method of reducing TNFa levels (e.g., protein or RNA levels) in a subject. In some embodiments, the method decreases TNFa levels (e.g., protein or RNA levels) in blood of the subject. In some embodiments, the method decreases TNFa levels (e.g., protein or RNA levels) in peripheral blood mononuclear cells (PBMCs) of the subject. In some embodiments, the reduction in cytokine levels is a statistically significant reduction, e.g., as described herein.

In certain aspects, provided herein is a method of reducing (e.g., causing a statistically significant reduction) levels (protein or RNA levels) of one or more cytokines (e.g., IL-1b, IL-2, IFNg, IL-4, IL-5, IL-6, IL-8, IL-10, TNFa and/or IL-17 levels) in a subject in need thereof, comprising administering to the subject a Prevotella histicola strain. In certain aspects, provided herein is a dose (e.g., a therapeutically effective dose) of a Prevotella histicola strain for use in reducing (e.g., causing a statistically significant reduction) levels (protein or RNA levels) of one or more cytokines (e.g., IL-1b, IL-2, IFNg, IL-4, IL-5, IL-6, IL-8, IL-10, TNFa and/or IL-17 levels) in a subject in need thereof. In certain aspects, provided herein is use of a dose (e.g., a therapeutically effective dose) of a Prevotella histicola strain for the preparation of a medicament for reducing (e.g., causing a statistically significant reduction) levels (protein or RNA levels) of one or more cytokines (e.g., IL-1b, IL-2, IFNg, IL-4, IL-5, IL-6, IL-8, IL-10, TNFa and/or IL-17 levels) in a subject in need thereof. In some embodiments, the Prevotella histicola strain is a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a bacterial composition (such as a pharmaceutical composition) and/or a solid dosage form. In some embodiments, the Prevotella histicola strain is Prevotella histicola Strain B (NRRL accession number B 50329). In certain aspects, provided herein is a method of reducing IL-1b, IL-2, IFNg, IL-4, IL-5, IL-6, IL-8, IL-10, TNFa and/or IL-17 levels (e.g., protein or RNA levels). In some embodiments, the method decreases IL-1b, IL-2, IFNg, IL-4, IL-5, IL-6, IL-8, IL-10, TNFa and/or IL-17 levels (e.g., protein or RNA levels) in blood of the subject. In some embodiments, the method decreases IL-1b, IL-2, IFNg, IL-4, IL-5, IL-6, IL-8, IL-10, TNFa and/or IL-17 levels (e.g., protein or RNA levels) in peripheral blood mononuclear cells (PBMCs) of the subject. In certain aspects, provided herein is a method of reducing IL-6, IL-8, and/or TNFa levels (e.g., protein or RNA levels). In some embodiments, the method decreases IL-6, IL-8, and/or TNFa levels (e.g., protein or RNA levels) in blood of the subject. In some embodiments, the method decreases IL-6, IL-8, and/or TNFa levels (e.g., protein or RNA levels) in peripheral blood mononuclear cells (PBMCs) of the subject. In certain aspects, provided herein is a method of reducing IL-6 levels (e.g., protein or RNA levels). In some embodiments, the method decreases IL-6 levels (e.g., protein or RNA levels) in blood of the subject. In some embodiments, the method decreases IL-6 levels (e.g., protein or RNA levels) in peripheral blood mononuclear cells (PBMCs) of the subject. In certain aspects, provided herein is a method of reducing IL-8 levels (e.g., protein or RNA levels). In some embodiments, the method decreases IL-8 levels (e.g., protein or RNA levels) in blood of the subject. In some embodiments, the method decreases IL-8 levels (e.g., protein or RNA levels) in peripheral blood mononuclear cells (PBMCs) of the subject. In certain aspects, provided herein is a method of reducing TNFa levels (e.g., protein or RNA levels). In some embodiments, the method decreases TNFa levels (e.g., protein or RNA levels) in blood of the subject. In some embodiments, the method decreases TNFa levels (e.g., protein or RNA levels) in peripheral blood mononuclear cells (PBMCs) of the subject. In some embodiments, the reduction in cytokine levels is a statistically significant reduction, e.g., as described herein.

In certain aspects, provided herein is a method of deepening a response in a post-dosing period in a subject in need thereof, comprising administering to the subject a Prevotella histicola strain prior to the post-dosing period, wherein a deepening of response is determined by improvement in the Psoriasis Area and Severity Index (PASI) score (e.g., as described herein). In certain aspects, provided herein is a dose (e.g., a therapeutically effective dose) of a Prevotella histicola strain for use in deepening a response in a post-dosing period in a subject in need thereof. In certain aspects, provided herein is use of a dose (e.g., a therapeutically effective dose) of a Prevotella histicola strain for the preparation of a medicament for deepening a response in a post-dosing period in a subject in need thereof. In some embodiments, the Prevotella histicola strain is administered for 8, 12, or 16 weeks prior to the post-dosing period. In some embodiments, the Prevotella histicola strain is administered for 16 weeks prior to the post-dosing period. In some embodiments, the subject is a responder, wherein a responder is a subject achieved at least a 50% reduction in their Psoriasis Area and Severity Index (PASI) scores from baseline at week 16 after administration of the Prevotella histicola strain. In some embodiments, the deepening of response is in the 8, 12, 16, 20, 24, or 28 week post-dosing period. In some embodiments, the deepening of response is in the 24 week post-dosing period. In some embodiments, the deepening of response is a change in response from over PASI 50 to PASI 75 or better. In some embodiments, the deepening of response is in the 24 week post-dosing period from over PASI 50 to PASI 75 or better. In some embodiments, the Prevotella histicola strain is a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a bacterial composition (such as a pharmaceutical composition) and/or a solid dosage form. In some embodiments, the Prevotella histicola strain is Prevotella histicola Strain B (NRRL accession number B 50329).

In certain aspects, provided herein is a method of maintaining a PASI-50 or greater (e.g., as described herein) in a post-dosing period in a subject in need thereof, comprising administering to the subject a Prevotella histicola strain prior to the post-dosing period. In certain aspects, provided herein is a dose (e.g., a therapeutically effective dose) of a Prevotella histicola strain for use in maintaining a PASI-50 or greater (e.g., as described herein) in a post-dosing period in a subject in need thereof. In certain aspects, provided herein is use of a dose (e.g., a therapeutically effective dose) of a Prevotella histicola strain for the preparation of a medicament for maintaining a PASI-50 or greater (e.g., as described herein) in a post-dosing period in a subject in need thereof. In some embodiments, the Prevotella histicola strain is administered for 8, 12, or 16 weeks prior to the post-dosing period. In some embodiments, the Prevotella histicola strain is administered for 16 weeks prior to the post-dosing period. In some embodiments, the subject is a responder, wherein a responder is a subject achieved at least a 50% reduction in their Psoriasis Area and Severity Index (PASI) scores from baseline at week 16 after administration of the Prevotella histicola strain. In some embodiments, the maintaining a PASI-50 or greater is in the 8, 12, 16, 20, 24, or 28 week post-dosing period. In some embodiments, the maintaining a PASI-50 or greater is in the 24 week post-dosing period. In some embodiments, the maintaining a PASI-50 or greater is in the 24 week post-dosing period. In some embodiments, the Prevotella histicola strain is a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a bacterial composition (such as a pharmaceutical composition) and/or a solid dosage form. In some embodiments, the Prevotella histicola strain is Prevotella histicola Strain B (NRRL accession number B 50329).

In certain aspects, provided herein is a method of decreasing the likelihood of rebound (e.g., as compared to placebo) in a post-dosing period in a subject in need thereof, comprising administering to the subject a Prevotella histicola strain prior to the post-dosing period. In certain aspects, provided herein is a dose (e.g., a therapeutically effective dose) of a Prevotella histicola strain for use in decreasing the likelihood of rebound (e.g., as compared to placebo) in a post-dosing period in a subject in need thereof. In certain aspects, provided herein is use of a dose (e.g., a therapeutically effective dose) of a Prevotella histicola strain for the preparation of a medicament for decreasing the likelihood of rebound (e.g., as compared to placebo) in a post-dosing period in a subject in need thereof. In some embodiments, the Prevotella histicola strain is administered for 8, 12, or 16 weeks prior to the post-dosing period. In some embodiments, the Prevotella histicola strain is administered for 16 weeks prior to the post-dosing period. In some embodiments, the subject is a responder, wherein a responder is a subject achieved at least a 50% reduction in their Psoriasis Area and Severity Index (PASI) scores from baseline at week 16 after administration of the Prevotella histicola strain. As used herein, “rebound” refers to an increase in PASI score to 125% of baseline value or above, or onset of new pustular erythrodermic psoriasis within 3 months of cessation of study treatment. In some embodiments, the Prevotella histicola strain is a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a bacterial composition (such as a pharmaceutical composition) and/or a solid dosage form. In some embodiments, the Prevotella histicola strain is Prevotella histicola Strain B (NRRL accession number B 50329).

In some embodiments, the Prevotella histicola is Prevotella Strain B 50329 (NRRL accession number B 50329; Strain B). In some embodiments, the Prevotella strain is a strain comprising at least at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain B 50329.

In some embodiments, the bacterial composition comprises one strain of bacteria, wherein the one strain of bacteria is a strain comprising at least 99.9% sequence identity to the nucleotide sequence of the Prevotella histicola Strain B 50329 (NRRL accession number B 50329). In some embodiments, the bacterial composition comprises one strain of bacteria, wherein the one strain of bacteria is the Prevotella histicola Strain B 50329 (NRRL accession number B 50329).

In some embodiments, the bacterial composition comprises at least 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola, e.g., of Prevotella Strain B 50329.

In some embodiments, the bacterial composition comprises at most 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola, e.g., of Prevotella Strain B 50329.

In some embodiments, the bacterial composition comprises about 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola, e.g., of Prevotella Strain B 50329.

In some embodiments, the bacterial composition comprises about 50 mg to about 600 mg of Prevotella histicola, e.g., of Prevotella Strain B 50329.

In some embodiments, the bacterial composition comprises about 600 mg to about 3 g of Prevotella histicola, e.g., of Prevotella Strain B 50329.

In some embodiments, the bacterial composition comprises about 55 mg of Prevotella histicola, e.g., of Prevotella Strain B 50329.

In some embodiments, the bacterial composition comprises about 550 mg of Prevotella histicola, e.g., of Prevotella Strain B 50329.

In some embodiments, the bacterial composition comprises about 2.76 g of Prevotella histicola, e.g., of Prevotella Strain B 50329.

In some embodiments, the bacterial composition comprises about 1.6×10¹⁰ total cells of Prevotella histicola, e.g., of Prevotella Strain B 50329.

In some embodiments, the bacterial composition comprises about 8×10¹⁰ total cells of Prevotella histicola, e.g., of Prevotella Strain B 50329.

In some embodiments, the bacterial composition comprises about 1.6×10¹¹ total cells of Prevotella histicola, e.g., of Prevotella Strain B 50329.

In some embodiments, the bacterial composition comprises about 3.2×10¹¹ total cells of Prevotella histicola, e.g., of Prevotella Strain B 50329.

In some embodiments, the bacterial composition comprises about 8×10¹¹ total cells of Prevotella histicola, e.g., of Prevotella Strain B 50329.

In some embodiments, the bacterial composition comprises about 1.6×10¹⁰ to about 8×10¹¹ total cells of Prevotella histicola, e.g., of Prevotella Strain B 50329.

In some embodiments, the bacterial composition comprises about 1.6×10¹⁰ to about 1.6×10¹¹ total cells of Prevotella histicola, e.g., of Prevotella Strain B 50329.

In some embodiments, the bacterial composition comprises about 1.6×10¹¹ to about 8×10¹¹ total cells of Prevotella histicola, e.g., of Prevotella Strain B 50329.

In some embodiments, the bacterial composition comprises about 8×10¹⁰ to about 8×10¹¹ total cells of Prevotella histicola, e.g., of Prevotella Strain B 50329.

In certain embodiments, the bacterial composition (e.g., composition of the total dose administered, e.g., once or twice daily) comprises at least 1×10¹⁰ total cells (e.g., at least 1×10¹⁰ total cells, at least 2×10¹⁰ total cells, at least 3×10¹⁰ total cells, at least 4×10¹⁰ total cells, at least 5×10¹⁰ total cells, at least 6×10¹⁰ total cells, at least 7×10¹⁰ total cells, at least 8×10¹⁰ total cells, at least 9×10¹⁰ total cells, at least 1×10¹¹ total cells of the Prevotella bacteria. In some embodiments, the bacterial composition comprises no more than 9×10¹¹ total cells (e.g., no more than 1×10¹⁰ total cells, no more than 2×10¹⁰ total cells, no more than 3×10¹⁰ total cells, no more than 4×10¹⁰ total cells, no more than 5×10¹⁰ total cells, no more than 6×10¹⁰ total cells, no more than 7×10¹⁰ total cells, no more than 8×10¹⁰ total cells, no more than 9×10¹⁰ total cells, no more than 1×10¹¹ total cells, no more than 2×10¹¹ total cells, no more than 3×10¹¹ total cells, no more than 4×10¹¹ total cells, no more than 5×10¹¹ total cells, no more than 6×10¹¹ total cells, no more than 7×10¹¹ total cells, no more than 8×10¹¹ total cells) of the Prevotella bacteria. In some embodiments, the bacterial composition comprises about 6×10⁹ total cells of the Prevotella bacteria. In some embodiments, the bacterial composition comprises about 1.6×10¹⁰ total cells of the Prevotella bacteria. In some embodiments, the bacterial composition comprises about 8×10¹⁰ total cells of the Prevotella bacteria. In some embodiments, the bacterial composition comprises about 1.6×10¹¹ total cells the Prevotella bacteria. In some embodiments, the bacterial composition comprises about 3.2×10¹¹ total cells the Prevotella bacteria. In some embodiments, the bacterial composition comprises about 8×10¹¹ total cells of the Prevotella bacteria. In some embodiments, the bacterial composition comprises about 1.6×10¹⁰ to about 8×10¹¹ total cells of the Prevotella bacteria. In some embodiments, the bacterial composition comprises about 1.6×10¹⁰ to about 1.6×10¹ total cells of the Prevotella bacteria. In some embodiments, the bacterial composition comprises about 8×10¹⁰ to about 8×10¹¹ total cells of the Prevotella bacteria. In some embodiments, the bacterial composition comprises about 1.6×10¹¹ to about 8×10¹¹ total cells of the Prevotella bacteria.

In certain embodiments, provided herein are solid dosage forms comprising the Prevotella bacteria. In some embodiments, the solid dosage form comprises an enteric coating (e.g., HPMC coat). In some embodiments, the solid dosage form is a capsule, e.g., an enteric coated capsule (e.g., HPMC coat). In some embodiments, each capsule comprises about 8×10¹⁰ total cells of the Prevotella bacteria. In some embodiments, 1, 2, 3, 4, 5, 6, 7, 8, 9, or 10 capsules are administered, e.g., once or twice daily to a subject. In some embodiments, 1 capsule (e.g., comprising about 8×10¹⁰ total cells) is administered, e.g., once or twice daily to a subject. In some embodiments, 2 capsules (e.g., each comprising about 8×10¹⁰ total cells) are administered, e.g., once or twice daily to a subject. In some embodiments, 4 capsules (e.g., each comprising about 8×10¹⁰ total cells) are administered, e.g., once or twice daily to a subject. In some embodiments, 10 capsules (e.g., each comprising about 8×10¹⁰ total cells) are administered, e.g., once or twice daily to a subject. In some embodiments, 1 capsule (e.g., comprising about 1.6×10¹¹ total cells) is administered, e.g., once or twice daily to a subject. In some embodiments, 2 capsules (e.g., each comprising about 1.6×10¹¹ total cells) are administered, e.g., once or twice daily to a subject. In some embodiments, 5 capsules (e.g., each comprising about 1.6×10¹¹ total cells) are administered, e.g., once or twice daily to a subject. In some embodiments, the Prevotella bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, the solid dosage form comprises a capsule. In some embodiments, the capsule is an enteric coated capsule (e.g., HPMC coated). In some embodiments, the capsule comprises about 8×10¹⁰ total cells of the Prevotella bacteria (e.g., total dose of a capsule or plurality of capsules). In some embodiments, the capsule comprises about 1.6×10¹¹ total cells of the Prevotella bacteria (e.g., total dose of a capsule or plurality of capsules). In some embodiments, the capsule comprises about 3.2×10¹¹ total cells of the Prevotella bacteria (e.g., total dose of a capsule or plurality of capsules). In some embodiments, the capsule comprises about 8×10¹¹ total cells of the Prevotella bacteria (e.g., total dose of a capsule or plurality of capsules). In some embodiments, the Prevotella bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, the solid dosage form comprises a tablet. In some embodiments, the tablet is an enteric coated tablet. In some embodiments, the enteric coated tablet is from 5 mm to 17 mm in diameter. In some embodiments, the tablet comprises about 8×10¹⁰ total cells of the Prevotella bacteria (e.g., total dose of a tablet or plurality of tablets). In some embodiments, the tablet comprises about 1.6×10¹¹ total cells of the Prevotella bacteria (e.g., total dose of a tablet or plurality of tablets). In some embodiments, the tablet comprises about 3.2×10¹¹ total cells of the Prevotella bacteria (e.g., total dose of a tablet or plurality of tablets). In some embodiments, the tablet comprises about 8×10¹¹ total cells of the Prevotella bacteria (e.g., total dose of a tablet or plurality of tablets). In some embodiments, the Prevotella bacteria in the tablet are lyophilized.

In certain embodiments, provided herein are solid dosage forms comprising the Prevotella bacteria. In some embodiments, the solid dosage form is a tablet, e.g., an enteric coated tablet. In some embodiments, the enteric coating comprises HPMC. In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P). In some embodiments, each tablet comprises about 8×10¹⁰ total cells of the Prevotella bacteria. In some embodiments, 1, 2, 3, 4, 5, 6, 7, 8, 9, or 10 tablets are administered, e.g., once or twice daily to a subject. In some embodiments, 1 tablet (e.g., comprising about 8×10¹⁰ total cells) is administered, e.g., once or twice daily to a subject. In some embodiments, 2 tablets (e.g., each comprising about 8×10¹⁰ total cells) are administered, e.g., once or twice daily to a subject. In some embodiments, 4 tablets (e.g., each comprising about 8×10¹⁰ total cells) are administered, e.g., once or twice daily to a subject. In some embodiments, 10 tablets (e.g., each comprising about 8×10¹⁰ total cells) are administered, e.g., once or twice daily to a subject. In some embodiments, each tablet comprises about 1.6×10¹¹ total cells of the Prevotella bacteria. In some embodiments, 1, 2, 3, 4, 5, 6, 7, 8, 9, or 10 tablets are administered, e.g., once or twice daily to a subject. In some embodiments, 1 tablet (e.g., comprising about 1.6×10¹¹ total cells) is administered, e.g., once or twice daily to a subject. In some embodiments, 2 tablets (e.g., each comprising about 1.6×10¹¹ total cells) are administered, e.g., once or twice daily to a subject. In some embodiments, 5 tablets (e.g., each comprising about 1.6×10¹¹ total cells) are administered, e.g., once or twice daily to a subject. In some embodiments, the Prevotella bacteria in the tablet are lyophilized (e.g., in a powder). In some embodiments, the Prevotella bacteria in the tablet are lyophilized in a powder, and the powder further comprises mannitol, magnesium stearate, and/or colloidal silicon dioxide.

In some embodiments, the solid dosage form comprises a mini-tablet. In some embodiments, the mini-tablet is enteric coated. In some embodiments, the mini-tablet is from 1 mm to 4 mm in diameter. In some embodiments, the mini-tablet (e.g., enteric coated mini-tablet) is a 1 mm mini-tablet, 1.5 mm mini-tablet, 2 mm mini-tablet, 3 mm mini-tablet, or 4 mm mini-tablet. In some embodiments, the solid dosage form comprises mini-tablets that comprise about 8×10¹⁰ total cells of the Prevotella bacteria (e.g., total dose of a plurality of mini-tablets). In some embodiments, the solid dosage form comprises mini-tablets that comprise about 1.6×10¹¹ total cells of the Prevotella bacteria (e.g., total dose of a plurality of mini-tablets). In some embodiments, the solid dosage form comprises mini-tablets that comprise about 3.2×10¹¹ total cells of the Prevotella bacteria (e.g., total dose of a plurality of mini-tablets). In some embodiments, the solid dosage form comprises mini-tablets that comprise about 8×10¹¹ total cells of the Prevotella bacteria (e.g., total dose of a plurality of mini-tablets). In some embodiments, the Prevotella bacteria in the mini-tablets are lyophilized.

In some embodiments, the mini-tablets (e.g., enteric coated mini-tablets) are contained in a capsule. In some embodiments, the capsule is a size 00, size 0, size 1, size 2, size 3, size 4, or size 5 capsule. In some embodiments, the capsule comprises (e.g., is coated with) a non-enteric coating (e.g., gelatin). In some embodiments, the capsule comprises a non-enteric coating. In some embodiments, the capsule comprises gelatin. In some embodiments, the mini-tablets (e.g., enteric coated mini-tablets) that comprise about 8×10¹¹ total cells of the Prevotella bacteria are contained in a capsule(s), wherein optionally the capsule comprises gelatin.

In some embodiments, the bacterial composition comprising Prevotella bacteria is prepared as a powder (e.g., for resuspension or for use in a solid dose form (such as a capsule)) or as a solid dose form, such as a tablet, a mini-tablet, a capsule, a pill, or a powder; or a combination of these forms (e.g., mini-tablets comprised in a capsule). The powder can comprise lyophilized bacteria. In some embodiments, the powder further comprises mannitol, magnesium stearate, and/or colloidal silicon dioxide.

In some embodiments, the bacterial composition is administered orally. In some embodiments, the administration to the subject once daily. In some embodiments, the bacterial composition is administered in 2 or more doses (e.g., 3 or more, 4 or more or 5 or more doses). In some embodiments, the administration to the subject of the two or more doses are separated by at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days.

In some embodiments, the bacterial composition is administered once daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In some embodiments, the bacterial composition is administered once daily for 1, 2, 3, 4, 5, 6, 7, 8, 9, 10, 11, 12, 13, 14, 15, 16, or 20 weeks. In some embodiments, the bacterial composition is administered once daily for 8 weeks. In some embodiments, the bacterial composition is administered once daily for 12 weeks. In some embodiments, the bacterial composition is administered once daily for 16 weeks. In some embodiments, the bacterial composition is administered once daily for 20 weeks. In some embodiments, the bacterial composition is administered once daily for at least 1, 2, 3, 4, 5, 6, 7, 8, 9, 10, 11, 12, 13, 14, 15, 16, or 20 weeks. In some embodiments, the bacterial composition is administered once daily for at least 8 weeks. In some embodiments, the bacterial composition is administered once daily for at least 12 weeks. In some embodiments, the bacterial composition is administered once daily for at least 16 weeks. In some embodiments, the bacterial composition is administered once daily for at least 20 weeks.

In some embodiments, the bacterial composition comprises lyophilized Prevotella bacteria. In certain embodiments, the lyophilized Prevotella bacteria are formulated into a solid dose form, such as a tablet, a mini-tablet, a capsule, a pill, or a powder. In some embodiments, the lyophilized Prevotella bacteria are contained in a capsule. In some embodiments, the powder further comprises mannitol, magnesium stearate, and/or colloidal silicon dioxide. In some embodiments, the lyophilized Prevotella bacteria are resuspended in a solution.

In some embodiments, the bacterial composition is formulated as a capsule or a tablet. In some embodiments, the bacterial formulation (e.g., composition) comprises an enteric coating or micro encapsulation. In some embodiments, the capsule is an enteric coated capsule. In some embodiments, the enteric coating allows the bacterial composition to be released in the upper small intestine, e.g., duodenum. In some embodiments, the enteric coating comprises HPMC.

In some embodiments, the subject is a mammal. In some embodiments, the subject is a human. In some embodiments, the subject is a non-human mammal (e.g., a dog, a cat, a cow, a horse, a pig, a donkey, a goat, a camel, a mouse, a rat, a guinea pig, a sheep, a llama, a monkey, a gorilla or a chimpanzee).

In some embodiments, the human subject is at least 18 years old. In some embodiments, the human subject is no more than 60 years old. In certain embodiments, the human subject has a body mass index of at least 18 kg/m². In some embodiments, the human subject has a body mass index of no more than 35 kg/m². In some embodiments, the human subject has not received live attenuated vaccination within 10 weeks prior to dosing. In some embodiments, the human subject does not require treatment with an anti-inflammatory drug. In some embodiments, the human subject does not have an active infection. In some embodiments, the human subject has not had an infection requiring antibiotic treatment within 6 weeks prior to dosing. In some embodiments, the human subject does not have renal or liver impairment. In some embodiments, the human subject does not have neoplastic disease or a history of neoplastic disease within 5 years prior to dosing. In some embodiments, the human subject has not had a major surgery within 4 weeks prior to dosing. In some embodiments, the human subject does not have impaired cardiac function or clinically significant cardiac diseases. In some embodiments, the human subject does not have a known history of human immunodeficiency virus (HIV), active hepatitis A, hepatitis B, or hepatitis C, and/or is not known to be positive for HCV ribonucleic acid and/or HBV surface antigen. In some embodiments, the human subject does not have an active central nervous system (CNS) malignancy. In some embodiments, the human subject does not have GI tract disease. In some embodiments, the human subject does not have a history of hypersensitivity or allergies to Prevotella (or Prevotella-containing probiotics) including, e.g., any associated excipients. In some embodiments, the human subject does not have a history of hypersensitivity or allergies to placebo capsule (magnesium stearate and cellulose) and/or to the hard capsule shells (hydroxyl propyl methyl cellulose and titanium dioxide). In some embodiments, the human subject does not have a significant history of drug abuse or regular use of illicit drugs or a history of alcohol abuse within 1 year prior to dosing. In some embodiments, the human subject does not have a clinically significant illness other than the immunoinflammatory disorder.

In some embodiments, the human subject has psoriasis. In some embodiments, the human subject has a confirmed diagnosis of mild to moderate plaque-type psoriasis for at least 6 months involving no more than 10% of body surface area (BSA) (excluding the scalp). In some embodiments, the human subject has a minimum of 2 psoriatic lesions. In some embodiments, the subject has not received systemic non-biologic psoriasis therapy (methotrexate [MTX], steroids, cyclophosphamide) or psoralen plus ultraviolet A (PUVA)/ultraviolet A (UVA) phototherapy within 4 weeks prior to dosing. In some embodiments, subject has not received treatment with biologic agents within 12 months prior to first dose. In some embodiments, the subject is not continuing use of topical or oral pharmacologically active agents 2 weeks prior to the start of dosing. In some embodiments, the human subject has a documented diagnosis of plaque psoriasis for ≥6 months.

In some embodiments, the human subject has had mild to moderate plaque psoriasis with plaque covering BSA of ≥3% and ≤10% and meet both of the following additional criteria: (i) PASI score of ≥6 and ≤15, and (ii) PGA score of 2 or 3.

In some embodiments, the method decreases the PASI (Psoriasis Area and Severity Index) score in the subject, e.g., after 16 weeks of treatment (e.g., as compared to the subject's PASI score prior to the commencement of treatment). In some embodiments, the method decreases the PASI (Psoriasis Area and Severity Index) score in the subject in the 8, 12, 16, 20, 24, or 28 week post-dosing period. In some embodiments, the method decreases the PASI (Psoriasis Area and Severity Index) score in the subject in the 24 week post-dosing period.

In some embodiments, the method increases a PASI percentage response rate (e.g., PASI-50, PASI-75, PASI-90, or PASI-100), e.g., as described herein. For example, the percentage of subjects who achieve a 75% or greater reduction in PASI score from baseline is represented by the PASI-75 value, e.g., after 16 weeks of treatment.

In some embodiments, the method decreases the LSS (Lesion Severity Score) in the subject, e.g., after 16 weeks of treatment (e.g., as compared to the subject's LSS prior to the commencement of treatment), e.g., as described herein.

In some embodiments, the method decreases the PGA (Physician's Global Assessment) score in the subject, e.g., after 16 weeks of treatment (e.g., as compared to the subject's PGA score prior to the commencement of treatment), e.g., as described herein.

In some embodiments, the method decreases the percent of BSA (Body Surface Area) involvement in the subject, e.g., after 16 weeks of treatment (e.g., as compared to the subject's percent involvement prior to the commencement of treatment), e.g., as described herein.

In some embodiments, the method decreases the mNAPSI (Modified Nail Psoriasis Severity Index) score in the subject, e.g., after 16 weeks of treatment (e.g., as compared to the subject's mNAPSI score prior to the commencement of treatment), e.g., as described herein.

In some embodiments, the method improves the DLQI (Dermatology Life Quality Index) score in the subject, e.g., after 16 weeks of treatment (e.g., as compared to the subject's DLQI score prior to the commencement of treatment), e.g., as described herein.

In some embodiments, the method improves the PSI (Psoriasis Symptom Inventory) score in the subject, e.g., after 16 weeks of treatment (e.g., as compared to the subject's PSI score prior to the commencement of treatment), e.g., as described herein.

In some embodiments, the method decreases pain in the subject, e.g., after 16 weeks of treatment (e.g., as compared to the subject's pain prior to the commencement of treatment), e.g., as described herein. For example, pain can be assessed by the SF-36 Bodily Pain Scale (SF-36 BPS) or the VAS Pain.

In some embodiments, the method decreases fatigue in the subject, e.g., after 16 weeks of treatment (e.g., as compared to the subject's fatigue prior to the commencement of treatment), e.g., as described herein.

In embodiments, the human subject has atopic dermatitis. In some embodiments, the human subject has a confirmed diagnosis of mild to moderate atopic dermatitis for at least 6 months involving a minimum of 3% to a maximum of 15% body surface area. In some embodiments, the subject has had a confirmed diagnosis of mild to moderate atopic dermatitis with an IGA score of 2 or 3. In some embodiments, the subject has at least 2 atopic dermatitis lesions with at least 1 in a site suitable for biopsy. In some embodiments, the subject is not receiving systemic non-biologic atopic dermatitis therapy (methotrexate (MTX), steroids, cyclophosphamide) or has received therapy within 4 weeks prior to dosing. In some embodiments, wherein the human subject is not receiving treatment with biologic agents within 12 months prior to first dose. In some embodiments, wherein the human subject is not continuing to use topical or oral pharmacologically active agents 2 weeks prior to the start of dosing.

In some embodiments, the method decreases the EASI (Eczema Area and Severity Index) score in the subject, e.g., after 16 weeks of treatment (e.g., as compared to the subject's EASI score prior to the commencement of treatment). In some embodiments, the method decreases the IGA (Investigator's Global Assessment) score in the subject, e.g., after 16 weeks of treatment (e.g., as compared to the subject's IGA score prior to the commencement of treatment). In some embodiments, the method decreases the SCORAD (SCORing Atopic Dermatitis) score in the subject, e.g., after 16 weeks of treatment (e.g., as compared to the subject's SCORAD score prior to the commencement of treatment).

In some aspects, the disclosure provides a bacterial composition described herein (e.g., in an amount described herein) for use in treating psoriasis (e.g., mild to moderate psoriasis) and/or atopic dermatitis (e.g., mild to moderate atopic dermatitis).

In certain embodiments, provided herein are methods of decreasing inflammation in a subject (e.g., a subject who has psoriasis (e.g., mild to moderate psoriasis) and/or atopic dermatitis (e.g., mild to moderate atopic dermatitis)) comprising administering to the subject a bacterial composition described herein, wherein administration of the bacterial composition decreases IL-23 levels (e.g., protein or RNA levels) in the subject. In some embodiments, the method decreases IL-23 RNA levels. In some embodiments, the method decreases IL-23 levels (e.g., protein or RNA levels) in the skin of the subject, e.g., in a skin lesion of the subject. For example, the lesion can be associated with psoriasis or atopic dermatitis.

In certain embodiments, provided herein are methods of decreasing inflammation in a subject (e.g., a subject who has psoriasis (e.g., mild to moderate psoriasis) and/or atopic dermatitis (e.g., mild to moderate atopic dermatitis)) comprising administering to the subject a bacterial composition described herein, wherein administration of the bacterial composition decreases IL-12b levels (e.g., protein or RNA levels) in the subject. In some embodiments, the method decreases IL-12b RNA levels. In some embodiments, the method decreases IL-12b levels (e.g., protein or RNA levels) in the skin of the subject, e.g., in a skin lesion of the subject. For example, the lesion can be associated with psoriasis or atopic dermatitis.

In certain embodiments, provided herein are methods of decreasing inflammation in a subject (e.g., a subject who has psoriasis (e.g., mild to moderate psoriasis) and/or atopic dermatitis (e.g., mild to moderate atopic dermatitis)) comprising administering to the subject a bacterial composition described herein, wherein administration of the bacterial composition decreases IL-17 levels (e.g., protein or RNA levels) in the subject. In some embodiments, the method decreases IL-17 RNA levels. In some embodiments, the method decreases IL-17 levels (e.g., protein or RNA levels) in the skin of the subject, e.g., in a skin lesion of the subject. For example, the lesion can be associated with psoriasis or atopic dermatitis.

In certain embodiments, provided herein are methods of decreasing inflammation in a subject (e.g., a subject who has psoriasis (e.g., mild to moderate psoriasis) and/or atopic dermatitis (e.g., mild to moderate atopic dermatitis)) comprising administering to the subject a bacterial composition described herein, wherein administration of the bacterial composition decreases IL-6 levels (e.g., protein or RNA levels) in the subject. In some embodiments, the method decreases IL-6 levels (e.g., protein or RNA levels) in PBMCs of the subject.

In certain embodiments, provided herein are methods of decreasing inflammation in a subject (e.g., a subject who has psoriasis (e.g., mild to moderate psoriasis) and/or atopic dermatitis (e.g., mild to moderate atopic dermatitis)) comprising administering to the subject a bacterial composition described herein, wherein administration of the bacterial composition decreases IL-8 levels (e.g., protein or RNA levels) in the subject. In some embodiments, the method decreases IL-8 levels (e.g., protein or RNA levels) in PBMCs of the subject.

In certain embodiments, provided herein are methods of decreasing inflammation in a subject (e.g., a subject who has psoriasis (e.g., mild to moderate psoriasis) and/or atopic dermatitis (e.g., mild to moderate atopic dermatitis)) comprising administering to the subject a bacterial composition described herein, wherein administration of the bacterial composition decreases TNF (TNFa) levels (e.g., protein or RNA levels) in the subject. In some embodiments, the method decreases TNF levels (e.g., protein or RNA levels) in PBMCs of the subject.

In some embodiments, the subject (e.g., a human subject) has Th1-mediated inflammation.

In some embodiments, the subject (e.g., a human subject) has Th2-mediated inflammation.

In some embodiments, the subject (e.g., a human subject) has Th17-mediated inflammation.

In certain embodiments, administration of the pharmaceutical composition results in decreased levels of the cytokine after 16 weeks of dosing in the subject. The decrease can be determined by measuring the decrease in the level of the cytokine after 16 weeks of dosing than the level of the cytokine at baseline.

### BRIEF DESCRIPTION OF THE DRAWINGS

FIG. 1 is three waterfall plots illustrating the decrease in IL-6, IL-8, and TNF (TNFa) production by subjects after 16 weeks of treatment with Prevotella histicola Strain B (Prevotella) or placebo. IL-6: left panel; IL-8: center panel; TNF: right panel.

FIG. 2 is a graph illustrating reduction of IL-23, IL-12b, and IL-17 RNA levels in skin biopsies from patients with PASI50+ responses following 16 weeks of treatment with Prevotella histicola Strain B. Baseline: open boxes and circles; 16 weeks of treatment: grey boxes and circles.

### DETAILED DESCRIPTION

Biomarker analysis supports gut-restricted action of Prevotella histicola Strain B to resolve inflammation throughout the body.

### Definitions

The term “about” when used before a numerical value indicates that the value may vary within a reasonable range, such as within +10%, +5%, or +1% of the stated value.

“Adjuvant” or “adjuvant therapy” broadly refers to an agent that affects an immunological or physiological response in a patient or subject. For example, an adjuvant might increase the presence of an antigen over time or help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition to a subject. Examples of routes of administration include oral administration, rectal administration, topical administration, inhalation (nasal) or injection. Administration by injection includes intravenous (IV), intramuscular (IM), and subcutaneous (SC) administration. The bacterial compositions described herein can be administered in any form by any effective route, including but not limited to oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In preferred embodiments, the bacterial compositions described herein are administered orally, rectally, topically, intravesically, by injection into or adjacent to a draining lymph node, intravenously, by inhalation or aerosol, or subcutaneously. In some preferred embodiments, the bacterial compositions described herein are administered orally.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

“Cellular augmentation” broadly refers to the influx of cells or expansion of cells in an environment that are not substantially present in the environment prior to administration of a composition and not present in the composition itself. Cells that augment the environment include immune cells, stromal cells, bacterial and fungal cells.

“Clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity. “Operational taxonomic units,” “OTU” (or plural, “OTUs”) refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. In 16S embodiments, OTUs that share ≥97% average nucleotide identity across the entire 16S or some variable region of the 16S are considered the same OTU (see e.g. Claesson M J, Wang Q, O'Sullivan O, Greene-Diniz R, Cole J R, Ros R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940.). In embodiments involving the complete genome, MLSTs, specific genes, or sets of genes OTUs that share ≥95% average nucleotide identity are considered the same OTU (see e.g. Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940.). OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Such characterization employs, e.g., WGS data or a whole genome sequence.

A “combination” of two or more monoclonal microbial strains includes the physical co-existence of the two monoclonal microbial strains, either in the same material or product or in physically connected products, as well as the temporal co-administration or co-localization of the monoclonal microbial strains.

The term “decrease” or “deplete” means a qualitative or quantitative difference between a reference and a value that is less than the reference, such that the difference is, depending on circumstances, at least 10%, 20%, 30%, 40%, 50%, 60%, 70%, 80%, or 90% of the reference, or such that the value is 1/100, 1/1000, 1/10,000, 1/100,000, 1/1,000,000 of the reference, or such that the value is undetectable after a treatment when compared to a reference representative of a pre-treatment state. Properties that may be decreased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size (e.g., in an animal tumor model)).

As used herein, “engineered bacteria” are any bacteria that have been genetically altered from their natural state by human intervention and the progeny of any such bacteria. Engineered bacteria include, for example, the products of targeted genetic modification, the products of random mutagenesis screens and the products of directed evolution.

The term “epitope” means a protein determinant capable of specific binding to an antibody or T cell receptor. Epitopes usually consist of chemically active surface groupings of molecules such as amino acids or sugar side chains. Certain epitopes can be defined by a particular sequence of amino acids to which an antibody is capable of binding.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

As used herein, the term “immune disorder” refers to any disease, disorder or disease symptom caused by an activity of the immune system, including autoimmune diseases, inflammatory diseases and allergies. Immune disorders include, but are not limited to, autoimmune diseases (e.g., Lupus, Scleroderma, hemolytic anemia, vasculitis, type one diabetes, Grave's disease, rheumatoid arthritis, multiple sclerosis, Goodpasture's syndrome, pernicious anemia and/or myopathy), inflammatory diseases (e.g., acne vulgaris, asthma, celiac disease, chronic prostatitis, glomerulonephritis, inflammatory bowel disease, pelvic inflammatory disease, reperfusion injury, rheumatoid arthritis, sarcoidosis, transplant rejection, vasculitis and/or interstitial cystitis), and/or an allergies (e.g., food allergies, drug allergies and/or environmental allergies).

“Immunotherapy” is treatment that uses a subject's immune system to treat disease (e.g., immune disease) and includes, for example, checkpoint inhibitors, cytokines, cell therapy, CAR-T cells, and dendritic cell therapy.

The term “increase” means a qualitative or quantitative difference between a reference and a value that is more than the reference, such that the difference is, depending on circumstances, at least 10%, 20%, 30%, 40%, 50%, 60%, 70%, 80%, 90%, 2-fold, 4-fold, 10-fold, 100-fold, 10\hat{ }3 fold, 10\hat{ }4 fold, 10\hat{ }5 fold, 10\hat{ }6 fold, and/or 10\hat{ }7 fold of the reference, e.g., where the difference is between a reference representative of a pre-treatment state and a value that is representative of a post-treatment state. Properties that may be increased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size (e.g., in an animal tumor model).

“Innate immune agonists” or “immuno-adjuvants” are small molecules, proteins, or other agents that specifically target innate immune receptors including Toll-Like Receptors (TLR), NOD receptors, RLRs, C-type lectin receptors, STING-cGAS Pathway components, inflammasome complexes. For example, LPS is a TLR-4 agonist that is bacterially derived or synthesized and aluminum can be used as an immune stimulating adjuvant. Immuno-adjuvants are a specific class of broader adjuvant or adjuvant therapy. Examples of STING agonists include, but are not limited to, 2′3′-cGAMP, 3′3′-cGAMP, c-di-AMP, c-di-GMP, 2′2′-cGAMP, and 2′3′-cGAM(PS)2 (Rp/Sp) (Rp, Sp-isomers of the bis-phosphorothioate analog of 2′3′-cGAMP). Examples of TLR agonists include, but are not limited to, TLR1, TLR2, TLR3, TLR4, TLR5, TLR6, TLR7, TLR8, TLR9, TLR10 and TLR11. Examples of NOD agonists include, but are not limited to, N-acetylmuramyl-L-alanyl-D-isoglutamine (muramyldipeptide (MDP)), gamma-D-glutamyl-meso-diaminopimelic acid (iE-DAP), and desmuramylpeptides (DMP).

The term “isolated” or “enriched” encompasses a microbe, bacteria or other entity or substance that has been (1) separated from at least some of the components with which it was associated when initially produced (whether in nature or in an experimental setting), and/or (2) produced, prepared, purified, and/or manufactured by the hand of man. Isolated microbes may be separated from at least about 10%, about 20%, about 30%, about 40%, about 50%, about 60%, about 70%, about 80%, about 90%, or more of the other components with which they were initially associated. In some embodiments, isolated microbes are more than about 80%, about 85%, about 90%, about 91%, about 92%, about 93%, about 94%, about 95%, about 96%, about 97%, about 98%, about 99%, or more than about 99% pure, e.g., substantially free of other components. The terms “purify,” “purifying,” and “purified” refer to a microbe or other material that has been separated from at least some of the components with which it was associated either when initially produced or generated (e.g., whether in nature or in an experimental setting), or during any time after its initial production. A microbe or a microbial population may be considered purified if it is isolated at or after production, such as from a material or environment containing the microbe or microbial population, and a purified microbe or microbial population may contain other materials up to about 10%, about 20%, about 30%, about 40%, about 50%, about 60%, about 70%, about 80%, about 90%, or above about 90% and still be considered “isolated.” In some embodiments, purified microbes or microbial population are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components thereof are generally purified from residual habitat products.

“Metabolite” as used herein refers to any and all molecular compounds, compositions, molecules, ions, co-factors, catalysts or nutrients used as substrates in any cellular or microbial metabolic reaction or resulting as product compounds, compositions, molecules, ions, co-factors, catalysts or nutrients from any cellular or microbial metabolic reaction.

“Microbe” refers to any natural or engineered organism characterized as a bacterium, fungus, microscopic alga, protozoan, and the stages of development or life cycle stages (e.g., vegetative, spore (including sporulation, dormancy, and germination), latent, biofilm) associated with the organism.

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state microbiome. The microbiome may be native to the subject or patient, or components of the microbiome may be modulated, introduced, or depleted due to changes in health state or treatment conditions (e.g., antibiotic treatment, exposure to different microbes). In some aspects, the microbiome occurs at a mucosal surface. In some aspects, the microbiome is a gut microbiome.

A “microbiome profile” or a “microbiome signature” of a tissue or sample refers to an at least partial characterization of the bacterial makeup of a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present or absent in a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present in a sample. In some embodiments, the microbiome profile indicates the relative or absolute amount of each bacterial strain detected in the sample.

“Modified” in reference to a bacteria broadly refers to a bacteria that has undergone a change from its wild-type form. Examples of bacterial modifications include genetic modification, gene expression, phenotype modification, formulation, chemical modification, and dose or concentration. Examples of improved properties are described throughout this specification and include, e.g., attenuation, auxotrophy, homing, or antigenicity. Phenotype modification might include, by way of example, bacteria growth in media that modify the phenotype of a bacterium that increase or decrease virulence.

Unless specifically stated or obvious from context, as used herein, the term “or” is understood to be inclusive (i.e., a composition “comprising elements A, B, or C” would not exclude a composition containing both elements A and B, a composition containing both elements B and C, a composition containing both elements A and C, or a composition containing all of elements A, B, and C). Unless specifically stated or obvious from context, as used herein, the terms “a,” “an,” and “the” are understood to be singular or plural.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), micro RNA (miRNA), silencing RNA (siRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 165, OTUs that share ≥97% average nucleotide identity across the entire 16S or some variable region of the 16S are considered the same OTU. See e.g. Claesson M J, Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940. For complete genomes, MLSTs, specific genes, other than 16S, or sets of genes OTUs that share ≥95% average nucleotide identity are considered the same OTU. See e.g., Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940. OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic clade are provided herein.

As used herein, a substance is “pure” if it is substantially free of other components. The terms “purify,” “purifying” and “purified” refer to a microbe or other material that has been separated from at least some of the components with which it was associated either when initially produced or generated (e.g., whether in nature or in an experimental setting), or during any time after its initial production. A microbe may be considered purified if it is isolated at or after production, such as from one or more other bacterial components, and a purified microbe or microbial population may contain other materials up to about 10%, about 20%, about 30%, about 40%, about 50%, about 60%, about 70%, about 80%, about 90%, or above about 90% and still be considered “purified.” In some embodiments, purified microbes are more than about 80%, about 85%, about 90%, about 91%, about 92%, about 93%, about 94%, about 95%, about 96%, about 97%, about 98%, about 99%, or more than about 99% pure. Bacterial compositions and the microbial components thereof are, e.g., purified from residual habitat products.

“Residual habitat products” refers to material derived from the habitat for microbiota within or on a subject. For example, microbes live in feces in the gastrointestinal tract, on the skin itself, in saliva, mucus of the respiratory tract, or secretions of the genitourinary tract (i.e., biological matter associated with the microbial community). Substantially free of residual habitat products means that the microbial composition no longer contains the biological matter associated with the microbial environment on or in the human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms. Substantially free of residual habitat products may also mean that the microbial composition contains no detectable cells from a human or animal and that only microbial cells are detectable. In one embodiment, substantially free of residual habitat products may also mean that the microbial composition contains no detectable viral (including microbial viruses (e.g., phage)), fungal, mycoplasmal contaminants. In another embodiment, it means that fewer than 1×10⁻²%, 1×10⁻³%, 1×10⁻⁴%, 1×10⁻⁵%, 1×10⁻⁶%, 1×10⁻⁷%, 1×10⁻⁸% of the viable cells in the microbial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹) such as through multiple 10-fold serial dilutions. This can further be confirmed by showing that multiple isolated colonies have similar cell shapes and Gram staining behavior. Other methods for confirming adequate purity include genetic analysis (e.g., PCR, DNA sequencing), serology and antigen analysis, enzymatic and metabolic analysis, and methods using instrumentation such as flow cytometry with reagents that distinguish desired constituents from contaminants.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein). Alternatively, specific binding applies more broadly to a two component system where one component is a protein, lipid, or carbohydrate or combination thereof and engages with the second component which is a protein, lipid, carbohydrate or combination thereof in a specific way.

The terms “subject” or “patient” refers to any animal. A subject or a patient described as “in need thereof” refers to one in need of a treatment for a disease. Mammals (i.e., mammalian animals) include humans, laboratory animals (e.g., primates, rats, mice), livestock (e.g., cows, sheep, goats, pigs), and household pets (e.g., dogs, cats, rodents). For example, the subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

As used herein, the term “treating” a disease in a subject or “treating” a subject having or suspected of having a disease refers to subjecting the subject to a pharmaceutical treatment, e.g., the administration of one or more agents, such that at least one symptom of the disease is decreased or prevented from worsening. Thus, in one embodiment, “treating” refers inter alia to delaying progression, expediting remission, inducing remission, augmenting remission, speeding recovery, increasing efficacy of or decreasing resistance to alternative therapeutics, or a combination thereof.

### Bacteria

In certain aspects, provided herein are bacterial compositions (e.g., pharmaceutical compositions) comprising Prevotella histicola useful for the treatment and/or prevention of inflammation (e.g., Th1-, Th2-, or Th17-mediated inflammation) and methods of using such bacterial compositions (e.g., for the treatment of inflammation), e.g., in a subject, e.g., in a human subject. In certain aspects, provided herein are bacterial compositions (e.g., pharmaceutical compositions) comprising Prevotella histicola useful for the treatment and/or prevention of psoriasis (e.g., mild to moderate psoriasis) and/or atopic dermatitis (e.g., mild to moderate atopic dermatitis) and methods of using such bacterial compositions (e.g., for the treatment of psoriasis, for the treatment of atopic dermatitis), e.g., in a subject, e.g., in a human subject. In some embodiments, the bacterial compositions comprise whole Prevotella histicola bacteria (e.g., live bacteria, killed bacteria, and/or attenuated bacteria). In some embodiments, the Prevotella histicola bacteria are non-viable. In some embodiments, the bacterial composition (e.g., pharmaceutical composition) comprises only one strain of bacteria, e.g., Prevotella histicola.

In some embodiments, the Prevotella histicola is Prevotella Strain B 50329 (NRRL accession number B 50329, also referred to as “Prevotella histicola Strain B” or “Prevotella Strain B”). In some embodiments, the Prevotella strain is a strain comprising at least at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain B 50329.

Prevotella histicola Strain B can be cultured according to methods known in the art. For example, Prevotella histicola can be grown in ATCC Medium 2722, ATCC Medium 1490, or other medium using methods disclosed, for example in Caballero et al., 2017. “Cooperating Commensals Restore Colonization Resistance to Vancomycin-Resistant Enterococcus faecium” Cell Host & Microbe 21:592-602, which is hereby incorporated by reference in its entirety.

In some embodiments, the bacterial compositions comprise whole Prevotella histicola bacteria (e.g., live bacteria, killed bacteria, and/or attenuated bacteria). In some embodiments, the Prevotella histicola bacteria are non-viable.

In some embodiments, the bacterial composition comprises about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², and/or 1×10¹³ total cells of Prevotella histicola.

In some embodiments, the bacterial composition comprises at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², or 1×10¹³ total cells of Prevotella histicola.

In some embodiments, the bacterial composition comprises at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², or 1×10¹³ total cells of Prevotella histicola.

In some embodiments, the bacterial composition comprises from about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ to about 8×10¹¹ total cells of Prevotella histicola.

In some embodiments, the bacterial composition comprises from about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10′⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹, 2.7×10¹¹, 2.8×10¹¹, 2.9×10¹¹, 3×10¹, 3.1×10¹¹, 3.2×10¹¹, 3.3×10¹¹, 3.4×10¹¹, 3.5×10¹, 3.6×10¹¹, 3.7×10¹¹, 3.8×10¹¹, 3.9×10¹¹, 4×10¹¹ to about 1×10¹² total cells of Prevotella histicola.

In some embodiments, the bacterial composition comprises about 1.6×10¹⁰ total cells of Prevotella histicola, e.g., of Prevotella Strain B 50329.

In some embodiments, the bacterial composition comprises about 8×10¹⁰ total cells of Prevotella histicola, e.g., of Prevotella Strain B 50329.

In some embodiments, the bacterial composition comprises about 1.6×10¹¹ total cells of Prevotella histicola, e.g., of Prevotella Strain B 50329.

In some embodiments, the bacterial composition comprises about 3.2×10¹¹ total cells of Prevotella histicola, e.g., of Prevotella Strain B 50329.

In some embodiments, the bacterial composition comprises about 8×10¹¹ total cells of Prevotella histicola, e.g., of Prevotella Strain B 50329.

In some embodiments, the bacterial composition comprises about 1.6×10¹⁰ to about 8×10¹¹ total cells of Prevotella histicola, e.g., of Prevotella Strain B 50329.

In some embodiments, the bacterial composition comprises about 1.6×10¹⁰ to about 1.6×10¹¹ total cells of Prevotella histicola, e.g., of Prevotella Strain B 50329.

In some embodiments, the bacterial composition comprises about 1.6×10¹¹ to about 8×10¹¹ total cells of Prevotella histicola, e.g., of Prevotella Strain B 50329.

In some embodiments, the bacterial composition comprises about 8×10¹⁰ to about 8×10¹¹ total cells of Prevotella histicola, e.g., of Prevotella Strain B 50329.

In some embodiments, the Prevotella bacteria may be quantified based on total cells, e.g., total cell count (TCC) (e.g., determined by Coulter counter).

In some embodiments, the bacterial composition comprises at least 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola.

In some embodiments, the bacterial composition comprises at most 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola.

In some embodiments, the bacterial composition comprises about 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola.

In some embodiments, the bacterial composition comprises about 1 g, 2 g, 2.5 g, 2.6 g, 2.61 g, 2.62 g, 2.63 g, 2.64 g, 2.65 g, 2.66 g, 2.67 g, 2.68 g, 2.69 g, 2.70 g, 2.71 g, 2.72 g, 2.73 g, 2.74 g, 2.75 g, 2.76 g, 2.77 g, 2.78 g, 2.79 g, 2.80, 2.81 g, 2.82 g, 2.83 g, 2.84 g, 2.85 g, 2.86 g, 2.87 g, 2.88 g, 2.89 g, 2.90 g, 3 g, 4 g, 5 g, 10 g, 20 g, 30 g, 40 g, or 50 g of Prevotella histicola.

In some embodiments, the bacterial composition is administered orally. In some embodiments, the administration to the subject once daily. In some embodiments, the bacterial composition is administered in 2 or more doses (e.g., 3 or more, 4 or more or 5 or more doses). In some embodiments, the administration to the subject of the two or more doses are separated by at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days.

In some embodiments, the bacterial composition is administered once daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

In some embodiments, the bacterial composition is administered once daily for 1, 2, 3, 4, 5, 6, 7, 8, 9, 10, 11, 12, 13, 14, 15, 16, or 20 weeks. In some embodiments, the bacterial composition is administered once daily for at least 1, 2, 3, 4, 5, 6, 7, 8, 9, 10, 11, 12, 13, 14, 15, 16, or 20 weeks.

In some embodiments, the bacterial composition is formulated as a capsule or a tablet. In some embodiments, the bacterial formulation (e.g., composition) comprises an enteric coating or micro encapsulation. In some embodiments, the capsule is an enteric coated capsule. In some embodiments, the enteric coating allows release of the bacterial composition in the small intestine, e.g., in the upper small intestine, e.g., in the duodenum. In some embodiments, the enteric coating comprises HTPMC.

In some embodiments, the subject is a mammal. In some embodiments, the subject is a human. In some embodiments, the subject is a non-human mammal (e.g., a dog, a cat, a cow, a horse, a pig, a donkey, a goat, a camel, a mouse, a rat, a guinea pig, a sheep, a llama, a monkey, a gorilla, or a chimpanzee).

In some embodiments, the Prevotella histicola bacteria are of a strain of Prevotella bacteria comprising one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more) proteins listed in Table 1 and/or one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more) genes encoding proteins listed in Table 1. In some embodiments, the Prevotella bacteria comprises all of the proteins listed in Table 1 and/or all of the genes encoding the proteins listed in Table 1.

TABLE 1
Exemplary Prevotella proteins

| Seq. | | | |
|---|---|---|---|
| ID. No. | Name | Uniprot ID | Amino Acid Sequence |
| 1 | Cluster: | G6ADE1 | MNLKTFTKTVLCFALFAVSAITAKAADHLAIVGE |
| | Uncharacterized | | AVWGGWDLVKATAMVKSPNNPDVFMATVHLN |
| | protein | | AGKGFKFLTEREWGKLEYRSGASDVVLKSGIRYK |
| | | | LYASIGASEDGKFKVSESANYEIICDLARKTVEVK |
| | | | KVAYQAKEIRYAALWMIGDATAGDWDYNNGVL |
| | | | LSQDSGNPTCYTATVELKEGEFKFTTNKQWGYD |
| | | | HSVYIFRDVNDQNKIVFGGEDNKWRITEDGMYN |
| | | | VTVDVPTKTISIKQIDDPAGHKPQFGNDVILVGDA |
| | | | TIAGWNLDNAIYLEHTGQAGRVFKTTTYLEAGKG |
| | | | FKFLSMLSYDDIDYRPANNTVLNPGVPGTFVPSLP |
| | | | SSTDTKFSVERSGNYDIVCNMNNRTVVVTLSENQ |
| | | | VLVNYPALWLIGSATSAGWNPGKAVELKRSEAD |
| | | | PAVYTARVQLKKGEFKILTSKNVGFDQPTYYRDS |
| | | | TNEHRIVFGVDGDEVAKKDCKWTLSENAEGTYD |
| | | | VTVDIEAMTIFCDKVNMDEPSVESTDKELILIGDA |
| | | | TYSAWDLPKSIVMTPVGPTTFKAVTHLEAGKEFK |
| | | | FLTELAWKRYEYRAESLRKELQEGSMSMLVPYR |
| | | | YTNDKDDKDHDFKFVVKESGNYEIVCDLYIPALII |
| | | | RKVRYQDTPVTYSSLWIVGSATPGGWTIERGIKM |
| | | | TQDENYPTKFTAKANLVPGELKFATNKFADFTQD |
| | | | FFFRGKDDYTAVLGGNDNKWNITEAGTYSVTIDV |
| | | | ASKRVTITKPARNAPTGISTVDSSDEAPAEYFTLN |
| | | | GIKVTTPSSGIYIKRQGGRTTKVVMK |
| 2 | Nicotinamide_ | P24520 | MDTYQILDIIGCIVGLIYIYQEYKASIWLWMTGIIM |
| | riboside_ | | PVIYMFVYYEAGLYADFGMQIYYTLAAIYGYLY |
| | transporter_PnuC | | WKLGKKKGTEDKEIPITHFPRRYIIPAIIVFFVLWIA |
| | | | LYYILICFTNSTVPVLDSFGNALSFIGLWALAKKY |
| | | | LEQWWIWIVVDAELSALYIYKGIPFTAMLYALYT |
| | | | VIAVAGYFKWRRYIKQQK |
| 3 | Pectate_ | Q8GCB2 | MRVRLYKNILLFLFLWVNTLACVSADTSRTVESQ |
| | trisaccharide-lyase | | PIENGLIITESKGWLETIYAKWKPVAEADGYYVY |
| | | | VKGGQYADYSKVDSELIRVYNGYVRVDIPGLKA |
| | | | GTYSLKIVAVKGGKETQSSEVTGLKVLNYVREGF |
| | | | AHKNYSGVGAYNDDGTLKSGAVVIYVNKDNAK |
| | | | TVSAHLGKTTFIGLQAILNAYQKGNITTPLSVRILG |
| | | | LLRNGDTDTFGSSTEGIQIKGKQADSEMNITIEGIG |
| | | | EDASIYGFGFLVRNAKSVEFRNLGIMRAMDDGVS |
| | | | LDTNNSNIWIHHMDLFYGKASGGDHIKGDGSIDV |
| | | | KTDSKYVTIDNCHFWDTGKTSMCGMKKETGPNY |
| | | | ITYHHNWFDHSDSRHARVRTMSVHLWNNYYDG |
| | | | CAKYGIGATMGCSVFSENNYFRATKNPILISKQGS |
| | | | DAKGTGKFSGEPGGMVKEYGSLFTEKGAESTYTP |
| | | | ISYADNNSSFDFYHAISRNEKVPASVKTLNGGNIY |
| | | | NNFDTDAALMYSYTPDATALVPSQVTGFYGAGR |
| | | | LNHGSLQFKFNNAVEDTNSTPIPALEALIDAYSGK |
| 4 | Glycosyl- | Q9AET5 | MKYNIAYCIEGFYNHGGMERILSVCANLLSDIYSI |
| | transferase_Gtf1 | | TIIVANQRGREHAYNLAQNVNVVDLGVSCKNYK |
| | | | EEYKKSLTRYLQDHQFSVVISLAGLELFFLPQIKD |
| | | | GSKKVMWFHFAFDVSKMFLSERFHGWKLNLLYY |
| | | | IHTIRRIYFAKKFDTIVVLSKSDCDSWSRFCNNVK |
| | | | YIYNPITIDRKVISNLSEESVIAVGRLGWQKGFDFL |
| | | | IDSWVLVDDKHPDWHLDIFGEGPDRLELQHQIDR |
| | | | KGLHDKVRLCGVTKQIEEEYGKHSIYVMSSRAEG |
| | | | FPLALLEASSCGLPMISFNCHQGPNEIIQEGENGFL |
| | | | VDKVGDIYTLSDRICKLIEDNNLRNMMGKKALDS |
| | | | SFRFEGEVIKKDWISLLKQLI |
| 5 | Cluster: Protein | A0A096B759 | MKRLFFMFLFLGTITMNSLAQEEKPIKYETKNFSL |
| | TonB | | PDKMPLYPGGDGALRAFLSLNLHYPEKAQAFGVE |
| | | | GRSLMKFCVSSDGSIKDISAVDCKITNYNRTEFNK |
| | | | LPLSKQESLKKECAKAFAKEAARVIRLMPKWEPA |
| | | | ELNGKKMNVYYSLPFTFKLR |
| 6 | Cluster: | G6AEN6 | MNYPLFIARKIYNGGDRTRKVSKPAIRIATIGVAIG |
| | Uncharacterized | | LAVMIISVGVVLGFKHTIRNKVVGFGSDITVANFL |
| | protein | | TLQSSEQYPIQITDSLVKSLQITPGIKHVQRYDYTQ |
| | | | GILKTDNDFLGVLLKGVGPDFDSTFIHENMVEGSL |
| | | | PHFHDNESQQKIVISKTIADKLNLKVGQRIFAYFIN |
| | | | KQGVRTRKFTITGIYATNMKQFDSQICFTDIYTTN |
| | | | KLNGWEPDQYSGAELQVDNFSQLTPISMRVLNKV |
| | | | KNTVDHYGGTYSSENIIEQNPQIFSWLDLMDMNV |
| | | | WIILALMISVAGVTMISGLLIIILERTQMIGILKALG |
| | | | SRNRQIRHIFLWFATFIIGKGLLWGNIIGLGCILFQS |
| | | | WTGLVKLDPQTYYVNTVPVEINIPLIIALNMVTML |
| | | | VCLVILIAPSYLISHIHPAKSMHYE |
| 7 | Bifunctional_(p) | P9WHG9 | MEDKFIYTDKERKLSYQILDELKDTLDKSFLENDL |
| | ppGpp_synthase/ | | PMLQVQLKDSVAKNTIHRNVFGLNPILCSLQTAAI |
| | hydrolase RelA | | AVKDIGLKRDSVIAILLHQSVQDGYITLEDIDNRF |
| | | | GKSVAKIIHGLIRIQTLYQKNPIIESENFRNLLLSFA |
| | | | EDMRVILIMIADRVNLMRQIRDAEDKEAQHKVAE |
| | | | EASYLYAPLAHKLGLYQLKRELEDLSLKYLEHDA |
| | | | YYLIKDKLNATKASRDAYINQFIAPVRERLTAGGL |
| | | | RFHIKGRTKSIHSIWQKMKKQKCGFEGIYDLFAIRI |
| | | | ILDAPLEKEKIQCWQAYSIVTDMYQPNPKRLRDW |
| | | | LSVPKSNGYECLHITVLGPEKKWVEVQIRTERMD |
| | | | EIAEHGLAAHWRYKGIKEEGGLDDWLASIRAALE |
| | | | AGDNLEVMDQFKSDLYEKEIYVFTPKGDLLKFPK |
| | | | GATILDFAYHIHSKVGNQCVGGKINAKNVSLRTE |
| | | | LHSGDTVEILTSATQKPKAEWLKIVKSSRAKAKIR |
| | | | LALKETQIKDGLYAKELLERRFKNKKIEIEESTMG |
| | | | HLLRKLGFKEVSEFYKQVADEKLDPNYIIEEYQK |
| | | | VYNHDHNLNQPKETESAENFEFENPTNEFLKKND |
| | | | DVLVIDKNLKGLDFSLAKCCHPIYGDPVFGFVTV |
| | | | NGGIKIHRTDCPNAPEMRKRFGYRIVKARWSGKG |
| | | | SSQYAITLRVIGNDDIGIVSNITNVISKDEKIVMRSI |
| | | | NIDSHDGLFSGNLVVLLDDNSKLNMLIKKLRTVK |
| | | | GVKQVTRI |
| 8 | Vitamin_B12_ | P06609 | MKRRIFLFVALSVSIVILFGLNLIIGSVHIPLSDILTI |
| | import_system_ | | LSGSFTGKESWRFIIWDSRLPQALTAMLCGSSLAV |
| | permease_protein_ | | CGLMLQTAFRNPLAGPDVFGISSGASLGVALVML |
| | BtuC | | LLGGTVETSMFTASGFLAILIVAFAGAILVTAFILF |
| | | | LSSVVRNSVLLLIVGIMVGYVASSAVTLLNFFSSE |
| | | | DGVKGYIVWGMGNFGGVSMSHIPLFAFLCLAGII |
| | | | ASFLLVKPLNILLLGPQYAESLGISIRRIRNILLVVV |
| | | | GILTAVTTAFCGPISFIGLAAPHVARLLFRTENHQK |
| | | | LLPGTLLVGTVVALLCNLICFLPRESGMIPLNAVT |
| | | | PLIGAPIIIYVIMKRH |
| 9 | NADH- | P33599 | MKLENKEFGFDSFATEMARLKNEKHFDYLVTVV |
| | quinone_ | | GEDFGTEEGLGCIYILENTSTHERCSVKQLAKKVG |
| | oxidoreductase_ | | EEFVIPSVIKLWADADLLEREVYDFYGIKFLGHPD |
| | subunit_C/D | | MRRLFLRNDFKGYPLRKDYDMDPAKNMYTTED |
| | | | DVELDTTTEWNLDKNGELVGTQHALFTDDNFVV |
| | | | NIGPQHPSTHGVLRLQTVLDGETVTNIYPHLGYIH |
| | | | RGIEKLCEQFTYPQTLALTDRMNYLSAMMNRHA |
| | | | LVGVIEEGMGIELSERILYIRTIMDELQRIDNHLLY |
| | | | TACCAQDLGALTAFLYGMRDREHVLNVMEETTG |
| | | | GRLIQNYYRIGGLQADIDPNFVSNVKELCKYLRP |
| | | | MIQEYVDVFGDNVITHQRFEGVGVMDEKDCISYG |
| | | | VTGPAGRASGWKNDVRKYHPYAMYDKVNFEEIT |
| | | | LTNGDSMDRYFCHIKEIYQSLNIIEQLIDNIPEGEFY |
| | | | IKQKPIIKVPEGQWYFSVEGASGEFGAYLDSRGDK |
| | | | TAYRLKFRPMGLTLVGAMDKMLRGQKIADLVTT |
| | | | GAALDFVIPDIDR |
| 10 | FKBP- | P45523 | MRTSTQSKDMGKKQEYKLRNEEFLHNISKKDSIK |
| | type_peptidyl- | | TLPHGIFYEIIKEGSGEGTVQPRSIVICNYRGSLISG |
| | prolyl_cis- | | QVFDDSWQKPTPEAFRLNELITGLQIALCAMHKG |
| | trans_isomerase | | DSWRIYIPYQEGYGSKRNADIPAFSTLIFDIELINIA |
| 11 | Putative_ | P9WKJ3 | MADNKIAKESVKREVIAGERLYTLLVYSENVAGV |
| | acetolactate_ | | LNQIAAVFTRRQVNIESLNVSASSIEGIHKYTITAW |
| | synthase_small_ | | SDAATIEKITKQVEKKIDVIKADYYEDSDLFIHEV |
| | subunit | | GLYKIATPILLENAEVSRAIRKRNARMMEVNPTYS |
| | | | TVLLAGMTDEVTALYHDLKNFDCLLQYSRSGRV |
| | | | AVTRGFSEPVSDFLKSEEESSVL |
| 12 | Serine/threonine_ | P0AGE4 | MKKKVKIGLLPRVIIAILLGIFFGYFMPTPLARVFL |
| | transporter_SstT | | TFNGIFSQFLGFMIPLIIIGLVTPAIADIGKGAGKLL |
| | | | LVTVIIAYVDTVVAGGLAYGTGLCLFPSMIASTGG |
| | | | AMPHIDKATELAPYFSINIPAMADVMSGLVFSFML |
| | | | GLGIAYGGLTATKNIFNEFKYVIEKVIAKAIIPLLPL |
| | | | YIFGVFLNMAHNGQAQQILLVFSQIIIVILVLHVFIL |
| | | | VYQFCIAGAIIRRNPFRLLWNMMPAYLTALGTSSS |
| | | | AATIPVTLEQTMKNGVGKEIAGFVVPLCATIHLSG |
| | | | SAMKITACALTICLLVGLPHDPALFIYFILMLSIIM |
| | | | VAAPGVPGGAIMAALAPLASILGFNSEAQALMIA |
| | | | LYIAMDSFGTACNVTGDGAIALVVNKMFGKKER |
| 13 | Cluster: | G6AJ07 | MKKLLLLVCAAVMSLSASAQAGDKALGAQLVFG |
| | Uncharacterized | | SETNSLGFGVKGQYYFTDHIRGEGSFDYFLKNKGI |
| | protein | | SMWDINANVHYLFDVADKFKVYPLAGLGYTNW |
| | | | SYKYEYAGAPVVEGSDGRLAVNLGGGVEYELTK |
| | | | NLNVNAEAKYQIISNYNQLVLGVGVAYKF |
| 114 | Heterocyst_differe | P22638 | MHFYCTKSSLDTMSERYVKRMIAKLASQGKTVIS |
| | ntiation_ATP- | | IAHRFSTIMDAKHIILLAKGKVVAEGTHQELLKTS |
| | binding_protein | | EDYRKLWSDQNDEID |
| 15 | UDP-2,3- | Q9I2V0 | MKNVYFLSDAHLGSLAIAHRRTQERRLVRFLDSI |
| | diacylglucosamine_ | | KHKASAVYLLGDMFDFWDEYKYVVPKGFTRFLG |
| | hydrolase | | KVSELTDMGVEVHFFTGNHDLWTYGYLEEECGV |
| | | | ILHRKPVTMEIYGKVFYLAHGDGLGDPDPMFQFL |
| | | | RKVFHNRVCQRLLNFFHPWWGMQLGLNWAKKS |
| | | | RLKRADGKEMPYLGEDKEYLVRYTKDYMRSHK |
| | | | DIDYYIYGHRHIELDLTLSGKVRMLILGDWIWQFT |
| | | | YAVFDGEHMFLEEYIEGESKP |
| 16 | Anaerobic_glycerol- | P0A9C0 | MNSKQNDNYDVIIIGGGITGAGTARDCALRGLKV |
| | 3- | | LLVEKFDFTNGATGRNHGLLHSGARYAVTDPESA |
| | phosphate_ | | TECIKENMVLRRIAKHCIEETDGLFITLPEDDINYQ |
| | dehydrogenase | | KTFVEACARAGISANIISPEEALRLDPSVNPDLLGA |
| | | | VRVPDASVDPFHLTTANVLDARQHGADVLTYHE |
| | | | VVAILTSNGRVEGVRLRNNHTGEEIEKHAVLVIN |
| | | | AAGIWGHDIAKMADIKINMFPAKGTLLVFGHRVN |
| | | | KMVINRCRKPANADILVPDDAVCVIGTTSDRVPY |
| | | | DTVDNLKITSEEVDTLIREGEKLAPSLATTRILRAY |
| | | | AGVRPLVAADNDPTGRSISRGIVCLDHEKRDGLT |
| | | | GMITITGGKMMTYRLMAEQATDLACKKLGINKT |
| | | | CETATTPLPGTAGKDSDNPHHTYSTAHKAAKGRQ |
| | | | GNRVKEIDERTEDDRALICECEEVSVGEAKYAIEE |
| | | | LHVHDLLNLRRRTRVGMGTCQGELCACRAAGV |
| | | | MCENGVKVDKAMTDLTKFINERWKGMRPVAWG |
| | | | STLDEAQLTTIIYQGLCGLGI |
| 17 | Anaerobic_glycerol- | P13033 | MRYDTIIIGGGLSGLTAGITLAKAGQKVCIVSAGQ |
| | 3- | | SSLHFHSGSFDLLGYDADGEVVTHPLQAIADLKA |
| | phosphate_ | | EHPYSKIGISNIEHLASQAKTLLCEAGISVMGNYE |
| | dehydrogenase | | QNHYRVTPLGTLKPAWLTTEGYAMIDDPEILPWK |
| | | | KVELLNIQGFMDFPTQFIAENLRMMGVECQIKTFT |
| | | | TDELSTARQSPTEMRATNIAKVLANKDALSKVSE |
| | | | RINAISGDPDALLLPAVLGFSNAESLDEMKQWIKK |
| | | | PVQYIATLPPSVSGVRTTILLKRLFAQAGGTLLIGD |
| | | | SATTGQFSGNHLVSITTDHLPDEKLYADHFILASG |
| | | | SFMSHGIRSNYAGVYEPVFKLDVDAAEKRDDWS |
| | | | VTNAFEAQPYMEFGVHTDKDFHATKDGKNIENL |
| | | | YAIGSVLSGHNSIKHADGTGVSLLTALYVAKKITG |
| | | | KG |
| 18 | Anaerobic_glycerol- | P0A996 | MAEGIQLKNISGNNLEQCLKCSICTAYCPVSAVEP |
| | 3- | | KYPGPKQSGPDQERYRLKDSKFFDEALKMCLNC |
| | phosphate_ | | KRCEVACPSGVRIADIIQASRITYSTHRPIPRDIMLA |
| | dehydrogenase | | NTDFVGTMANMVAPIVNATLGLKPVKAVLHGV |
| | | | MGIDKHRTFPAYSSQKFETWYKRMAAKKQDSYS |
| | | | KHVSYFHGCYVNYNFPQLGKDLVKIMNAVGYGV |
| | | | HLLEKEKCCGVALIANGLSGQARRQGKVNIRSIR |
| | | | KAAEQNRIVLTTSSTCTFTMRDEYEHLLDIKTDDV |
| | | | RENITLATRFLYRLIEKGDIKLAFRKDFKMRTAYH |
| | | | SACHMEKMGWIIYSTELLKMIPGLELIMLDSQCC |
| | | | GIAGTYGFKKENYQRSQEIGEGLFKQIKELNPDCV |
| | | | STDCETCKWQIEMSTGYEVKNPISILADALDVEET |
| | | | IKLNQ |
| 19 | Glycerol_uptake_ | P18156 | MMIKNIVLSIPISLIIYLNHLIMEYSMTTQFLMELIG |
| | facilitator_protein | | TLILVLFGDGVCACVTLNKSKGQKAGWVVITIAW |
| | | | GLAVCMGVLVAGPYTGAHLNPAVSIGLAVAGMF |
| | | | PWSSVPYYIVAQMIGGFLGGLLVWFFYKDHYDA |
| | | | TDDEAAKLGTFCTSPAIRNYKMNFLSEVIATLVLV |
| | | | FIIISFSVDGNTGDAEHFKFGLAALGPIPVTLLIIAL |
| | | | GMSLGGTTGYAMNPARDLSPRLAHAVCMKGDN |
| | | | DWSYSWIPVLGPIIGAIIAGFCGAALLLV |
| 20 | Serine/threonine- | Q97PA9 | MSEKIIPSNEPAQAASEPIKASYTEYTVIPSQGYCQ |
| | protein_kinase_ | | FVKCKKGDQPVVLKGLKEAYRERVLLRNALKRE |
| | StkP | | FKQCQRLNHPGIVRYQGLVDVEGYGLCIEEEYVD |
| | | | GRTLQAYLKESHTDDEKITIVNQIADALRYAHQQ |
| | | | GVAHRNLKPSNILITKQGDHVKLIDFNVLSLDDVK |
| | | | PTADTTRFMAPELKDETMTADGTADIYSLGTIMK |
| | | | VMGLTLAYSEVIKRCCAFKRSDRYSDIDEFLADEN |
| | | | HDGSSFSMPKIGKGTVVIGFIAVVVIALAALAYNY |
| | | | GGALVDQVGKIDVTSIFKSDAETAPEDSAMVKSV |
| | | | EQNNNDSVADEAPATGKLAFMNTMKPALYKDLD |
| | | | RLFAKHSDDRAKLNRAIKVYYRGLIQANDTLDNE |
| | | | QRAELDRVFGNYVKQKKAALK |
| 21 | Cluster: D-alanyl- | G6AHI1 | MLVAQLFVGVLQAQKPVQNRRQAVGQSMERQG |
| | D-alanine | | LVNVKAVVPSIKVALMYARTDNFCHRMALS |
| | dipeptidase | | |
| 22 | Anaerobic_C4- | P0ABN5 | MITGLVIIQLLIVLALIFIGARVGGIGLGIYGMIGVFI |
| | dicarboxylate_ | | LVYGFGLAPGSAPIDVMMIIVAVITAASALQASGG |
| | transporter_DcuA | | LEYLVGVAAKFLQKHPDHITYFGPITCWLFCVVA |
| | | | GTAHTSYSLMPIIAEIAQTNKIRPERPLSLSVIAASL |
| | | | GITCSPVSAATAALISQDLLGAKGIELGTVLMICIP |
| | | | TAFISILVAAFVENHIGKELEDDPEYKRRVAAGLI |
| | | | NPEAACEEVQKAENEHDPSAKHAVWAFLFGVAL |
| | | | VILFGFLPQLRPEGVSMSQTIEMIMMSDAALILLV |
| | | | GKGKVGDAVNGNIFKAGMNAVVAIFGIAWMGN |
| | | | TFYVGNEKILDAALSSMISSTPILFAVALFLLSIMLF |
| | | | SQAATVTTLYPVGIALGINPLLLIAMFPACNGYFF |
| | | | LPNYPTEVAAIDFDRTGTTRVGKYVINHSFQIPGFI |
| | | | TTIVSILLGVLMVQFFR |
| 23 | L-asparaginase_2 | P00805 | MRILKITFVTVLALVMSTVVFAQKPKIRIIATGGTI |
| | | | AGVSASATSSAYGAGQVGVQTLIDAVPQIKDIAD |
| | | | VSGEQLVNIGSQDMNDEVWLKLAKRINDLLNKE |
| | | | GYDGVLITHGTDTMEETAYFLSLTVHTDKPVVM |
| | | | VGSMRPSTAISADGPANLYNGICTLVDPSSKGHG |
| | | | VMVCMNNELFEAKSVIKTHTTDVSTFKGGLYGE |
| | | | MGYVYNGKPYFLHKPVAKQGLTSEFNVDNLTSL |
| | | | PKVGIVYGYANCSPLPIQAFVNAKFDGIVLAGVG |
| | | | DGNFYKDVFDVALKAQNSGIQIVRSSRVPFGPTNL |
| | | | NGEVDDAKYHFVASLNLNPQKARVLLMLALTKT |
| | | | KDWQKIQQYFNEY |
| 24 | Trehalose_ | P9WQ19 | MALACAMTMSASAQMGTNPKWLGDAIFYQIYPS |
| | synthase/amylase_ | | SYMDTDGNGIGDLPGITQKLDYIKSLGVNAIWLN |
| | TreS | | PVFESGWFDGGYDVIDFYKIDPRFGTNTDMVNLV |
| | | | KEAHKRGIKVCLDLVAGHTSTKCPWFKESANGD |
| | | | RNSRYSDYFIWTDSISEADKKEIAERHKEANPASS |
| | | | THGRYVEMNAKRGKYYEKNFFECQPALNYGFAK |
| | | | PDPNQPWEQPVTAPGPQAVRREMRNIMAFWFDK |
| | | | GVDGFRVDMASSLVKNDWGKKEVSKLWNEMRE |
| | | | WKDKNYPECVLISEWSDPAVAIPAGFNIDFMIHFG |
| | | | IKGYPSLFFDRNTPWGKPWPGQDISKDYKFCYFD |
| | | | KAGKGEVKEFVDNFSEAYNATKNLGYIAIPSANH |
| | | | DYQRPNIGTRNTPEQLKVAMTFFLTMPGVPFIYY |
| | | | GDEIGMKYQMDLPSKEGSNERAGTRTPMQWTSG |
| | | | PTAGFSTCNPSQLYFPVDTEKGKLTVEAQQNDPR |
| | | | SLLNYTRELTRLRHSQPALRGNGEWILVSKESQPY |
| | | | PMVYKRTSGGETVVVAINPSDKKVSANIAHLGKA |
| | | | KSLIMTGKASYKTGKTEDAVELNGVSAAVFKIAE |
| 25 | Ribitol-5- | Q720Y7 | MNIAVIFAGGSGLRMHTKSRPKQFLDLNGKPIIIYT |
| | phosphate_ | | LELFDNHPGIDAIVVACIESWIPFLEKQLRKFEINK |
| | cytidylyltransferase | | VVKIVPGGESGQASIYNGLCAAEAYIKSKNVASE |
| | | | DTTVLIHDGVRPLITEETITDNINKVAEVGSCITCIP |
| | | | ATETLVVKQHDGSLEIPSRADSLIARAPQSFLLSDI |
| | | | LTAHRRAIDEKKNDFIDSCTMMSHYGYRLGTIIGP |
| | | | MENIKITTPTDFFVLRAMVKVHEDQQIFGL |
| 26 | UDP-Glc: alpha-D- | B5L3F2 | MTEKKSVSIVLCTYNGTKYLQEQLDSILAQTYPLH |
| | GlcNAc- | | EIIIQDDGSTDNTWQILEKYEEKYPLIHIYHNEGTH |
| | diphos- | | GVNANFLSAMHRTTGDFIAIADQDDIWETDKIAN |
| | phoundecaprenol | | QMTTIGNKLLCSGLTRPFSSDGSFAYFDNRPRNVS |
| | | | IFRMMFLGLPGHTMLFRRELLRMMPPVTHSFFNV |
| | | | SLYDAALSILAASHDSIAFCNKVLVNFRRHADATT |
| | | | YNDYSRSLPSWQNGLYELLWGLRHYHQARSIALP |
| | | | IYRGKLALMEGITTNYHDFIEAKAIMRLETQKGL |
| | | | WAFLRLQYLLTKNHQRLFQTSGGSFIKMIRAWLY |
| | | | PVMQLYMYHHALRRCK |
| 27 | UDP-N- | P33038 | MESFIIEGGHRLSGTIAPQGAKNEALEVICATLLTT |
| | acetylglucosamine | | EEVIIRNIPNILDVNNLIKLLQDIGVKVKKLGANDF |
| | | | SFQADEVKLDYLESIDFVKKCSSLRGSVLMIGPLL |
| | | | GRFGKATIAKPGGDKIGRRRLDTHFLGFKNLGAR |
| | | | FVRIEDRDVYEIQADKLVGDYMLLDEASVTGTAN |
| | | | IIMSAVMAEGTTTIYNAACEPYIQQLCHLLNAMG |
| | | | AKITGIASNLITIEGVTSLHGAEHRILPDMIEVGSFI |
| | | | GMAAMVGDGVRIKDVSIPNLGLILDTFRRLGVQII |
| | | | EDEDDLIIPRQDHYVIDSFIDGTIMTISDAPWPGLT |
| | | | PDLISVLLVVATQAQGSVLFHQKMFESRLFFVDK |
| | | | LIDMGAQIILCDPHRAVVVGHDHAKKLRAGRMSS |
| | | | PDIRAGIALLIAALTAEGTSRIDNIAQIDRGYENIEG |
| | | | RLNALGAKVQRVEIC |
| 28 | Sensor_protein_ | P30855 | MERSGNFYKAIRLGYILISILIGCMAYNSLYEWQEI |
| | EvgS | | EALELGNKKIDELRKEINNINIQMIKFSLLGETILE |
| | | | WNDKDIEHYHARRMAMDSMLCRFKATYPAERID |
| | | | SVRHLLEDKERQMCQIVQILEQQQAINDKITSQVP |
| | | | VIVQKSVQEQPKKSKRKGFLGIFGKKEEAKPTVTT |
| | | | TMHRSFNRNMRTEQQAQSRRLSVHADSLAARNA |
| | | | ELNRQLQGLVVQIDGKVQTDLQKREAEITAMRER |
| | | | SFIQIGGLTGFVILLLVISYIIIHRNANRIKRYKQETA |
| | | | DLIERLQQMAKRNEALITSRKKAVHTITHELRTPL |
| | | | TAITGYAGLIQKNFNADKTGMYIRNIQQSSDRMR |
| | | | EMLNTLLSFFRLDDGKEQPNFSTCRISSIAHTLESE |
| | | | FMPIAINKGLALTVTNHTDAVVLTDKERILQIGNN |
| | | | LLSNAIKFTENGAVSLTMGYDNGMLKLIVKDTGS |
| | | | GMTEEEQQRVFGAFERLSNAAAKDGFGLGLSIVQ |
| | | | RIVTMLGGTIQLKSEKGKGSRFTVEIPMQSAEELP |
| | | | ERINKTQIHHNRTLHDIVAIDNDKVLLLMLKEMY |
| | | | AQEGIHCDTCTNAAELMEMIRRKEYSLLLTDLNM |
| | | | PDINGFELLELLRTSNVGNSRIIPIIVTTASGSCNRE |
| | | | ELLERGFSDCLLKPFSISELMEVSDKCAMKGKQN |
| | | | EKPDFSSLLSYGNESVMLDKLIAETEKEMQSVRD |
| | | | GEQRKDFQELDALTHHLRSSWEILRADQPLRELY |
| | | | KQLHGSAVPDYEALNNAVTAVLDKGSEIIRLAKE |
| | | | ERRKYENG |
| 29 | Phosphate- | Q7A5Q2 | MKRSRFYITVGLILSLTLLMSACGQKKAKDGRTD |
| | binding protein_ | | TPTSGTIKFASDESFSPIVEELLQNYQFRYPQAHLL |
| | PstS | | PIYTDDNTGMKLLLDQKVNLFITSHAMTKGEDAI |
| | | | LRGKGPIPEVFPIGYDGIAFIVNRSNPDSCITVDDV |
| | | | KKILQGKIAKWNQLNPKNNRGSIEVVFDNKASAT |
| | | | LHYVVDSILGGKNIKSENIVAAKNSKSVIDYVNKT |
| | | | PNAIGVIGSNWLNDHRDTTNTTFKKDVTVASISK |
| | | | ATVASPSNSWQPYQAYLLDGRYPFVRTIYALLAD |
| | | | PHKALPYAFANYIANPIGQMIIFKAGLLPYRGNINI |
| | | | REVEVKNQ |
| 30 | Bifunctional_ | P9WHM7 | MAGTKRIKTALISVFHKDGLDDLLKKLDEEGVQF |
| | purine_ | | LSTGGTQQFIESLGYECQKVEDVTSYPSILGGRVK |
| | biosynthesis_ | | TLHPKIFGGILARRDNEEDQKQMVEYTIPAIDLVIV |
| | protein_PurH | | DLYPFEQTVASGASAQDIIEKIDIGGISLIRAGAKN |
| | | | FKDVVIVPSKAEYPVLLQLLNTKGAETEIEDRKMF |
| | | | AERAFGVSSHYDTAIHSWFAAE |
| 31 | Multidrug_efflux_ | P0AE06 | MEEEKGGRIGQRPYILKIITERNYIIIIDMKKAKILL |
| | pump_subunit_ | | FVTALVAVLTSCGGGQKGLPTSDEYPVITIGASNA |
| | AcrA | | QLKTTYPATIKGVQDVEVRPKVSGFITKLNIHEGE |
| | | | YVHAGQVLFVIDNSTYQAAVRQAQAQVNSAQSA |
| | | | VAQAKANVVQANASLNSANAQAATSRLTYNNSQ |
| | | | NLYNNKVIGDYELQSAKNTYETAQASVRQAQSGI |
| | | | ASAQAAVKQAEAGVRQAQAMLSTAKDNLGFCY |
| | | | VKSPASGYVGSLPFKEDALVSASSAQPVTTISNTS |
| | | | TIEVYFSMTEADVLKLSRTDDGLSNAIKKFPAVSL |
| | | | LLADGSTYNHEGAIVKTSGMIDATTGTINVIARFP |
| | | | NPEHLLKSGGSGKIVIAKNNNRALLIPQEAVTQVQ |
| | | | NKMFVYKVDAKDKVHYSEITVDPQNDGINYIVTS |
| | | | GLKMGERIVSKGVSSLEDGAKIKALTPAEYEEAIK |
| | | | KAEKLGENQSSASGFLKTMKGDSK |
| 32 | Cell_division_ | Q81X30 | MAKRRNKARSHHSLQVVTLCISTAMVLILIGMVV |
| | protein_FtsX | | LTVFTSRNLSSYVKENLTVTMILQPDMSTEESAAL |
| | | | CQRIRSLHYINSLNFISKEQALKEGTRELGANPAEF |
| | | | AGQNPFTGEIELQLKANYANNDSIKNIERELRTYR |
| | | | GVSDITYPQNLVESVNHTLGKISLVLLVIAILLTIVS |
| | | | FSLMNNTIRLSIYARRFSIHTMKLVGASWGFIRAPF |
| | | | LRRAVMEGLVSALLAIAVLGVGLCLLYDYEPDIT |
| | | | KVLSWDVLVITAGVMLAFGVLIATFCSWLSVNKF |
| | | | LRMKAGDLYKI |
| 33 | Fe(2+)_transporter_ | Q9PMQ9 | MKLSDLKTGETGVIVKVLGHGGFRKRIIEMGFIQG |
| | FeoB | | KQVEVLLNAPLRDPVKYKIMGYEVSLRHSEADQI |
| | | | EVISAEEARQLEQAKADNEPQQGALSNNIPDESDH |
| | | | ALTPFELTDAANRKSKVINVALVGNPNCGKTSLF |
| | | | NFASGAHERVGNYSGVTVDAKVGRANYEGYEFH |
| | | | LVDLPGTYSLSAYSPEELYVRKQLVEKTPDVVINV |
| | | | IDASNLERNLYLTTQLIDMHVRMVCALNMFDETE |
| | | | QRGDNIDYQKISELFGIPMVPTVFTNGRGVKELFH |
| | | | QVIAVYEGKEDETSQFRHIHINHGHELEGGIKNIQ |
| | | | EHLRAYPDICQRYSTRYLAIKLLEHDKDVEELIKP |
| | | | LKDSDEIFKHRDIAAQRVKEETGNESETAIMDAK |
| | | | YGFIHGALEEADYSTGQKKDTYQTTHFIDQILINK |
| | | | YFGFPIFFLILFIMFTATFVIGQYPMDWIDGGVSWL |
| | | | GDFISSNMPDGPVKDMLVDGIIGGVGAVIVFLPQI |
| | | | LILYFFISYMEDSGYMARAAFIMDKLMHKMGLH |
| | | | GKSFIPLIMGFGCNVPAVMATRTIESRRSRLVTMLI |
| | | | LPLMSCSARLPIYVMITGSFFALKYRSLAMLSLYV |
| | | | IGILMSVIMSRVFSRFLVKGEDTPFVMELPPYRFPT |
| | | | WKAIGRHTWEKGKQYLKKMGGIILVASIIVWALG |
| | | | YFPLPDKPDMGQQERQEHSFIGQIGHAVEPVFRPQ |
| | | | GFNWKLDVGLLAGVGAKEIVASTMGVLYSNDDS |
| | | | FKDDNSFSSEGGKYVKLHKQITQDVANLHGVSYN |
| | | | EAEPIATLTAFCFLLFVLLYFPCIATIAAIKGETGS |
| | | | WGWALFAAGYTTLLAWVVSAIVFQVGMLFIG |
| 34 | Pneumolysin | Q04IN8 | MKKNLLKAVLPASLALFAVTFGSCSQDGQLTGTK |
| | | | EDTGERVLDNTREIQNYLRTLPLAPMMSRASDPV |
| | | | PSDDGTTVPVDEGTSKTEEKGVLNGIPGSWVKTT |
| | | | RRYKMTQAFDESFLFDPTSDIVYPGCVLKGGTIAN |
| | | | GTYAIITSHETGDVTFSINLSPANPQEARETSATVH |
| | | | NIRKSEYQEVWNKWANMQWKESPITTIESVEKIN |
| | | | SQEELATKLGVAVNSPVANGSLNFGFNFNKKKNH |
| | | | ILARLIQKYFSVSTDAPKKGNIFESIDKEALDGYQP |
| | | | VYISNINYGRIIYLSVESDEDEKVVDEAINFAMNQI |
| | | | KGVDVSVSADQSLHYRKVLANCDIRITVLGGGQT |
| | | | IQKEVLKGDIDSFQRFLNADIPMEQMSPISFSLRYA |
| | | | VDNSQARVVTSNEFTVTQRDFVPEFKKVRMQLQ |
| | | | VLGFSGTNTGPFPNLDREAGLWGSISLSLNGQDNE |
| | | | LVKISQSNPFFFNYREKKETMHPIGFGGIVTVEFD |
| | | | KDPNESLEDFVDHQKMTFVSDLHSTRSIYNYNFG |
| | | | RTTFTHTLGTLYTKYKGDDPIFVLESNNKNVKIHT |
| | | | YVKVLDMKFFN |
| 35 | Cluster: | G6AG77 | MTKFIYAMSLFLLAAISIKAQPIQKTSGCLLHGSV |
| | Uncharacterized | | VSSTDATAIAGATVRLYQLKKLVGGTVSDASGNF |
| | protein | | DVKCPSSGSLQLRITAVGFKEVDTTLNVPTVTPLSI |
| | | | YMRAGKHAMDEVTVTASEKRGMTSTTVIGQTA |
| | | | MEHLQPSSFADLLALLPGGMTKIPALGSANVITLR |
| | | | EAGPPSSQYATSSLGTKFVIDGQAIGTDANMQYIA |
| | | | GSFQGDADNSRNHVSYGVDMREIPTDNIEKVEVV |
| | | | RGIPSVKYGELTSGLINITRKRSQSPLLLRLKADEY |
| | | | GKLVSVGKGFLLSGKWNLNVDGGLLDARKEPRN |
| | | | RFETYRRLTFSARLRRKWNLGERYVLEWSGATD |
| | | | YSLNIDNVKTDPEIQIHREDSYRSSYLKMGMNHR |
| | | | LLLRRKALVGLQSVSLAYSASLASDRIHQTEAVA |
| | | | LQRDYVVPLAYEGGEYDGLFLPMQYLCDYRVEG |
| | | | KPFYSTLRGETEWLARTSFISHHITAGGEFLLNKN |
| | | | YGRGQIFDITKPLHASTARRPRSYKDIPATDILSFY |
| | | | AEDKATMPIGKHQLTVMAGLRTTQMLNIPASYA |
| | | | VHGKLFTDTRVNVQWDFPSFLGFKSFVSGGLGM |
| | | | MTKMPTVLDLYPDYVYKDITEMNYWDIRPAYKR |
| | | | IHIRTYKLNQVNPDLRPARNKKWEIRLGMDKGAH |
| | | | HFSVTYFHEDMKDGFRSTTTMRPFIYKRYDTSVIN |
| | | | PSALTGPPSLASLPVVTDTLLDGYGRTENGSRITK |
| | | | QGIEFQYSSPRIPVIQTRITVNGAWFRTLYENSIPLF |
| | | | RSAPNVVVGTVAIADRYAGYYMSTDKYDKQIFTS |
| | | | NFIFDSYVDKLGLILSATAECFWMSNTKRPATSST |
| | | | PMGYMDITGTVHPYVEADQSDPYLRWLVLTGTA |
| | | | GQDMDYRERSYMLVNFKATKRFGRHLSLSFFAD |
| | | | RVFYVAPDYEVNGFIVRRTFSPYFGMEIGLKI |
| 36 | Cell_division_ | P0A9R7 | MLIDFKKVNIYQDERLILKDIDFQATEGEFIYLIGR |
| | ATP- | | VGSGKSSLLKTFYGELDIDQEDAEKAEVLGESVL |
| | binding_protein_ | | DIKQKRIPALRRQMGIIFQDFQLLHDRSVAKNLKF |
| | FtsE | | VLQATGWKDKEKIKQRIKEVLEQVGMIDKAAKM |
| | | | PSELSGGEQQRIAIARAFLNNPKIILADEPTGNLDP |
| | | | ETASNIVSILKDTCKNGTTVIMSTHNINLLSQFPGK |
| | | | VYRCMEQALVPVTNEAQTKDLEEDSTSVEPLIEP |
| | | | VLEEEAQAEDSKE |
| 37 | Di- | P0C2U3 | MFENQPKALYALALANTGERFGYYTMIAVFALFL |
| | /tripeptide_ | | RANFGLEPGTAGLIYSIFLGLVYFLPLIGGIMADKF |
| | transporter | | GYGKMVTIGIIVMFAGYLFLSVPLGGGTVAFGAM |
| | | | LAALLLISFGTGLFKGNLQVMVGNLYDTPELASK |
| | | | RDSAFSIFYMAINIGALFAPTAAVKIKEWAETSLG |
| | | | YAGNDAYHFSFAVACVSLIVSMGIYYAFRSTFKH |
| | | | VEGGTKKTEKAAAAAVEELTPQQTKERIVALCLV |
| | | | FAVVIFFWMAFHQNGLTLTYFADEFVSPTSTGVQ |
| | | | SMAFDVVNLVMIVFIVYSIMALFQSKTTKAKGIAC |
| | | | AVILAAIAVLAYKYMNVNGQVEVSAPIFQQFNPF |
| | | | YVVALTPISMAIFGSLAAKGKEPSAPRKIAYGMIV |
| | | | AGCAYLLMVLASQGLLTPHEQKLAKAAGETVPF |
| | | | ASANWLIGTYLVLTFGELLLSPMGISFVSKVAPPK |
| | | | YKGAMMGGWFVATAIGNILVSVGGYLWGDLSLT |
| | | | VVWTVFIVLCLVSASFMFLMMKRLEKVA |
| 38 | Calcium- | Q47910 | MKKILIFVAGLCMSLAASAQIQRPKLVVGLVVDQ |
| | transporting_ | | MRWDYLYYYYNEYGTDGLRRLVDNGFSFENTHI |
| | ATPase | | NYAPTVTAIGHSSVYTGSVPAITGIAGNYFFQDDK |
| | | | NVYCCEDPNVKSVGSDSKEGQMSPHRLLASTIGD |
| | | | ELQISNDFRSKVIGVALKDRASILPAGHAADAAY |
| | | | WWDTSAGHFVTSTFYTDHLPQWVIDFNEKNHTA |
| | | | PNFNIKTSTQGVTMTFKMAEAALKNENLGKGKET |
| | | | DMLAVSISSTDAIGHVYSTRGKENHDVYMQLDK |
| | | | DLAHFLKTLDEQVGKGNYLLFLTADHGAAHNYN |
| | | | YMKEHRIPAGGWDYRQSVKDLNGYLQGKFGIAP |
| | | | VMAEDDYQFFLNDSLIAASGLKKQQIIDESVEYLK |
| | | | KDPRYLYVFDEERISEVTMPQWIKERMINGYFRG |
| | | | RSGEIGVVTRPQVFGAKDSPTYKGTQHGQPFPYD |
| | | | THIPFLLYGWNVKHGATTQQTYIVDIAPTVCAML |
| | | | HIQMPNGCIGTARNMALGN |
| 39 | Poly-beta-1,6-N- | Q5HKQ0 | MDRQVFQTDSRQRWNRFKWTLRVLITIAILLGVV |
| | acetyl-D- | | FVAMFALEGSPQMPFRHDYRSVVSASEPLLKDNK |
| | glucosamine_ | | RAEVYKSFRDFFKEQKMHSNYAKVAARQHRFVG |
| | synthase | | HTDNVTQKYIKEWTDPRMGIRSAWYVNWDKHA |
| | | | YISLKNNLKNLNMVLPEWYFINPKTDRIEARIDQR |
| | | | ALKLMRRAHIPVLPMLTNNYNSAFRPEAIGRIMR |
| | | | DSTKRMGMINELVAACKHNGFAGINLDLEELNIN |
| | | | DNALLVTLVKDFARVFHANGLYVTQAVAPFNED |
| | | | YDMQELAKYDDYLFLMAYDEYNAGSQAGPVSS |
| | | | QRWVEKATDWAAKNVPNDKIVLGMATYGYNW |
| | | | AQGQGGTTMSFDQTMATALNAGAKVNFNDDTY |
| | | | NLNFSYQDEDDGTLHQVFFPDAVTTFNIMRFGAT |
| | | | YHLAGFGLWRLGTEDSRIWKYYGKDLSWESAAR |
| | | | MPIAKIMQLSGTDDVNFVGSGEVLNVTSEPHAGRI |
| | | | GIVLDKDNQLIIEERYLSLPATYTVQRLGKCKEKQ |
| | | | LVLTFDDGPDSRWTPKVLSILKHYKVPAAFFMVG |
| | | | LQIEKNIPIVKDVFNQGCTIGNHTFTHHNMIENSD |
| | | | RRSFAELKLTRMLIESITGQSTILFRAPYNADADPT |
| | | | DHEEIWPMIIASRRNYLFVGESIDPNDWQQGVTA |
| | | | DQIYKRVLDGVHQEYGHIILLHDAGGDTREPTVT |
| | | | ALPRIIETLQREGYQFISLEKYLGMSRQTLMPPIKK |
| | | | GKEYYAMQANLSLAELIYHISDFLTALFLVFLVLG |
| | | | FMRLVFMYVLMIREKRAENRRNYAPIDPLTAPAV |
| | | | SIIVPAYNEEVNIVRTISNLKEQDYPSLKIYLVDDG |
| | | | SKDNTLQRVREVFENDDKVVIISKKNGGKASALN |
| | | | YGIAACSTDYIVCVDADTQLYKDAVSKLMKHFIA |
| | | | DKTGKLGAVAGNVKVGNQRNMLTYWQAIEYTT |
| | | | SQNFDRMAYSNINAITVIPGAIGAFRKDVLEAVGG |
| | | | FTTDTLAEDCDLTMSINEHGYLIENENYAVAMTE |
| | | | APESLRQFIKQRIRWCFGVMQTFWKHRASLFAPS |
| | | | KGGFGMWAMPNMLIFQYIIPTFSPIADVLMLFGLF |
| | | | SGNASQIFIYYLIFLLVDASVSIMAYIFEHESLWVL |
| | | | LWIIPQRFFYRWIMYYVLFKSYLKAIKGELQTWG |
| | | | VLKRTGHVKGAQTIS |
| 40 | ATP_synthase_ | P29707 | MSQINGRISQIIGPVIDVYFDTKGENPEKVLPNIYD |
| | subunit_beta, | | ALRVKKADGQDLIIEVQQQIGEDTVRCVAMDNTD |
| | sodium_ion_specific | | GLQRGLEVVPTGSPIVMPAGEQIKGRMMNVIGQPI |
| | | | DGMSALQMEGAYPIHREAPKFEDLSTHKEMLQT |
| | | | GIKVIDLLEPYMKGGKIGLFGGAGVGKTVLIMELI |
| | | | NNIAKGHNGYSVFAGVGERTREGNDLIRDMLESG |
| | | | VIRYGEKFRKAMDEGKWDLSLVDSEELQKSQAT |
| | | | LVYGQMNEPPGARASVALSGLTVAEEFRDHGGK |
| | | | NGEAADIMFFIDNIFRFTQAGSEVSALLGRMPSAV |
| | | | GYQPTLASEMGAMQERITSTKHGSITSVQAVYVP |
| | | | ADDLTDPAPATTFTHLDATTELSRKITELGIYPAV |
| | | | DPLGSTSRILDPLIVGKEHYDCAQRVKQLLQKYN |
| | | | ELQDIIAILGMDELSDDDKLVVNRARRVQRFLSQP |
| | | | FTVAEQFTGVKGVMVPIEETIKGFNAILNGEVDDL |
| | | | PEQAFLNVGTIEDVKEKAKQLLEATKA |
| 41 | Cluster: | G6AGX5 | MNPIYKIITSILFCVLSINTMAQDLTGHVTSKADDK |
| | Uncharacterized | | PIAYATVILKENRLYAFTDEKGNYTIKNVPKGKY |
| | protein | | TVVFSCMGYASQTVVVMVNAGGATQNVRLAED |
| | | | NLQLDEVQVVAHRKKDEITTSYTIDRKTLDNQQI |
| | | | MTLSDIAQLLPGGKSVNPSLMNDSKLTLRSGTLER |
| | | | GNASFGTAVEVDGIRLSNNAAMGETAGVSTRSVS |
| | | | ASNIESVEVVPGIASVEYGDLTNGVVKVKTRRGSS |
| | | | PFIVEGSINQHTRQIALHKGVDLGGNVGLLNFSIE |
| | | | HARSFLDAASPYTAYQRNVLSLRYMNVFMKKSL |
| | | | PLTLEVGLNGSIGGYNSKADPDRSLDDYNKVKDN |
| | | | NVGGNIHLGWLLNKRWITNVDLTAAFTYADRLS |
| | | | ESYTNESSNATQPYIHTLTEGYNIAEDYDRNPSAN |
| | | | IILGPTGYWYLRGFNDSKPLNYSLKMKANWSKAF |
| | | | GKFRNRLLVGGEWTSSMNRGRGTYYADMRYAPS |
| | | | WREYRYDALPSLNNIAIYAEDKLSMDVNERQNAE |
| | | | LTAGIREDITSIPGSEYGSVGSFSPRMNARYVFRFG |
| | | | QNSWLNSMTLHAGWGRSVKIPSFQVLYPSPSYRD |
| | | | MLAFASTSDADNRSYYAYYTYPSMARYNANLK |
| | | | WQRADQWDLGVEWRTKIADVSLSFFRSKVSNPY |
| | | | MATDVYTPFTYKYTSPAMLQRSGIAVADRRFSID |
| | | | PQTGIVTVSDASGVKSPVTLGYEERNTYVTNTRY |
| | | | VNADALQRYGLEWIVDFKQIKTLRTQVRLDGKY |
| | | | YHYKAQDETLFADVPVGLNTRQSDGRLYQYVGY |
| | | | YRGGAATTTNYTANASASNGSVSGQVDLNATITT |
| | | | HIPKIRLIVALRLESSLYAFSRATSSRGYVVSSGNE |
| | | | YFGVPYDDKTENQTVIVYPEYYSTWDAPDVLIPF |
| | | | AEKLRWAETNDRGLFNDLAQLVVRTNYPYTLNP |
| | | | NRLSAYWSANLSVTKEIGRHVSVSFYANNFFNTL |
| | | | SQVHSTQTGLETSLFGSGYVPSFYYGLSLRLKI |

In some embodiments, the Prevotella bacteria are of a strain of Prevotella bacteria free or substantially free of one or more (e.g., 1, 2, 3, 4, 5, 6, 7, 8, 9, 10, 11, 12, 13, 14, 15, 16, 17, 18, 19, 20, 21, 22, 23, 24, 25, or more) proteins listed in Table 2 and/or one or more (e.g., 1, 2, 3, 4, 5, 6, 7, 8, 9, 10, 11, 12, 13, 14, 15, 16, 17, 18, 19, 20, 21, 22, 23, 24, 25, or more) genes encoding proteins listed in Table 2. In some embodiments, Prevotella bacteria are free of all of the proteins listed in Table 2 and/or all of the genes encoding the proteins listed in Table 2.

TABLE 2
Other Prevotella proteins

| Seq. | | | |
|---|---|---|---|
| ID. No. | Name | Uniprot ID | Amino Acid Sequence |
| 42 | UDP-Gal: alpha-D- | Q03084 | MERIDISVLMAVYKKDNPAFLRESLESIFSQTVEA |
| | GlcNAc- | | AEVVLLEDGPLTDALYDVIKSYEAIYSTLKVVSYP |
| | diphosphoundecap | | ENRGLGKTLNDGLLLCKYNLVARMDADDICKPN |
| | renol | | RLEMEYNWLKSHEDYDVIGSWVDEFTDNKTRVK |
| | | | SIRKVPEAYDEIKNYAQYRCPINHPTAMYRKAAV |
| | | | LAVGGYLTEYFPEDYFLWLRMLNNGSKFYNIQES |
| | | | LLWFRYSEETVAKRGGWAYACDEVRILVRMLKM |
| | | | GYIPFHVFCQSVVIRFTTRVMPLPIRQRLYNLIRKT |
| 43 | ATP_synthase_ | A1B8P0 | MSQINGRISQIIGPVIDVYFDTKGENPEKVLPKIHD |
| | subunit_beta | | ALRVKRANGQDLIIEVQQHIGEDTVRCVAMDNTD |
| | | | GLQRNLEVVPTGSPIVMPAGDQIKGRMMNVIGQP |
| | | | IDGMEALSMEGAYPIHREAPKFEDLSTHKEMLQT |
| | | | GIKVIDLLEPYMKGGKIGLFGGAGVGKTVLIMELI |
| | | | NNIAKGHNGYSVFAGVGERTREGNDLIRDMLESG |
| | | | VIRYGEKFRKAMDEGKWDLSLVDQEELQKSQAT |
| | | | LVYGQMNEPPGARASVALSGLTVAEEFRDHGGK |
| | | | NGEAADIMFFIDNIFRFTQAGSEVSALLGRMPSAV |
| | | | GYQPTLASEMGTMQERITSTKHGSITSVQAVYVP |
| | | | ADDLTDPAPATTFTHLDATTELSRKITELGIYPAV |
| | | | DPLGSTSRILDPLIVGKDHYECAQRVKQLLQHYN |
| | | | ELQDIIAILGMDELSDEDKLVVNRARRVQRFLSQP |
| | | | FTVAEQFTGVKGVMVPIEETIKGFNAILNGEVDDL |
| | | | PEQAFLNVGTIEDVKEKAKRLLEATK |
| 44 | Cell_division_ | O05779 | MPIGNGQKYQLTIINHTEIIMLIDYKKVNIYQDERL |
| | ATP- | | ILKDVDFQAETGEFIYLIGRVGSGKSSLLKTIYGEL |
| | binding_protein_ | | DIDSEDAEKAVVLDESMPNIKRSRIPALRKQMGIIF |
| | FtsE | | QDFQLLHDRSVAKNLKFVLQATGWTSKQKIERRI |
| | | | EEVLAQVGMTDKKNKMPSELSGGEQQRIAIARAL |
| | | | LNTPKIIIADEPTGNLDPETAANIVSILKDSCQAGT |
| | | | TVIMSTHNINLIDQFPGKVYRCHEGELHQLTDKKE |
| | | | VSELAEETAPVETIDEPEQND |
| 45 | Hemin_transport_ | Q56992 | MKRNILLFICLATSILLLFGLNLTTGSVQIPFADILD |
| | system_permease_ | | ILCGRFIGKESWEYIILENRLPQTLTAILCGASLSVC |
| | protein_HmuU | | GLMLQTAFRNPLAGPDVFGISSGAGLGVALVMLL |
| | | | LGGTVSTSIFTVSGFLAILTAAFVGAIAVTALILFLS |
| | | | TLVRNSVLLLIVGIMVGYVSSSAVSLLNFFASEEG |
| | | | VKSYMVWGMGNFGAVSMNHIPLFSILCLIGIIASF |
| | | | LLVKPLNILLLGPQYAESLGISTRQIRNILLVVVGL |
| | | | LTAITTAFCGPISFIGLAIPHIARLLFRTENHQILLPG |
| | | | IVLSGAAIALLCNFICYLPGESGIIPLNAVTPLIGAPI |
| | | | IIYVIIQRR |
| 46 | Hexuronate_ | O34456 | MKKYYPWVLVALLWFVALLNYMDRQMLSTMQ |
| | transporter | | EAMKVDIAELNHAEAFGALMAVFLWIYGIVSPFA |
| | | | GIIADRVNRKWLVVGSIFVWSAVTYLMGYAESFD |
| | | | QLYWLRAFMGISEALYIPAALSLIADWHEGKSRSL |
| | | | AIGIHMTGLYVGQAVGGFGATLAAMFSWHAAFH |
| | | | WFGIIGIVYSLVLLLFLKENPKHGQKSVLQGETKP |
| | | | SKNPFRGLSIVFSTWAFWVILFYFAVPSLPGWATK |
| | | | NWLPTLFANSLDIPMSSAGPMSTITIAVSSFIGVIM |
| | | | GGVISDRWVQRNLRGRVYTSAIGLGLTVPALMLL |
| | | | GFGHSLVSVVGAGLCFGIGYGMFDANNMPILCQF |
| | | | ISSKYRSTAYGIMNMTGVFAGAAVTQVLGKWTD |
| | | | GGNLGNGFAILGGIVVLALVLQLSCLKPTTDNME |
| 47 | 1,4-alpha- | P9WN45 | MVTKKTTTKKAPVKKTSAKTTKVKEPSHIGLVKN |
| | glucan_branching_ | | DAYLAPYEDAIRGRHEHALWKMNQLTQNGKLTL |
| | enzyme_GlgB | | SDFANGHNYYGLHQTADGWVFREWAPNATEIYL |
| | | | VGDFNGWNEQEAYQCHRIEGTGNWELTLPHDAM |
| | | | QHGQYYKMRVHWEGGEGERIPAWTQRVVQDEA |
| | | | SKIFSAQVWAPAEPYVWEKKTFKPQTSPLLIYECH |
| | | | IGMAQDEEKVGTYNEFREKVLPRIIKDGYNAIQIM |
| | | | AIQEHPYYGSFGYHVSSFFAASSRFGTPEELKALID |
| | | | EAHKNGIAVIMDIVHSHAVKNEVEGLGNLAGDPN |
| | | | QYFYPGERHEHPAWDSLCFDYGKDEVLHFLLSNC |
| | | | KYWLEEYHFDGFRFDGVTSMLYYSHGLGEAFCN |
| | | | YADYFNGHQDDNAICYLTLANCLIHEVNKNAVTI |
| | | | AEEVSGMPGLAAKFKDGGYGFDYRMAMNIPDY |
| | | | WIKTIKELPDEAWKPSSIFWEIKNRRSDEKTISYCE |
| | | | SHDQALVGDKTIIFRLVDADMYWHFRKGDETEM |
| | | | THRGIALHKMIRLATIAAINGGYLNFMGNEFGHPE |
| | | | WIDFPREGNGWSHKYARRQWNLVDNEELCYHLL |
| | | | GDFDRKMLEVITSEKKFNETPIQEIWHNDGDQILA |
| | | | FSRGELVFVFNFSPSHSYSDYGFLVPEGSYNVVLN |
| | | | TDAREFGGFGFADDTVEHFTNSDPLYEKDHKGW |
| | | | LKLYIPARSAVVLRKK |
| 48 | Cluster: YihY | D9RW24 | MKIDIERIKYFLTVGMFMKTEHSSKRRNMLIRQFQ |
| | family protein | | KFYLTVKFFFVRDHAASTAQLSFSTIMAIVPIASMI |
| | | | FAIANGFGFGQFLEKQFREMLSAQPEAATWLLKL |
| | | | TQSYLVHAKTGLFIGIGLMIMLYSVFSLIRTVETTF |
| | | | DNIWQVKDSRPISRIVIDYTALMFLVPISIIILSGLSI |
| | | | YFYSFVENLNGLRFLGTIASFSLRYLVPWAILTLM |
| | | | FIVLYVFMPNAKVKITKTVAPAMIASIAMLCLQA |
| | | | VYIHGQIFLTSYNAIYGSFAALPLFMLWILASWYI |
| | | | CLFCAELCYFNQNLEYYECLIDTEDICHNDLLILC |
| | | | ATVLSHICQRFANDQKPQTALQIKTETHIPIRVMT |
| | | | DILYRLKEVNLISENFSPTSDEVTYTPTHDTNNITV |
| | | | GEMIARLESTPASDFALLGFSPKKAWNHDIYDRV |
| | | | GSIREIYLNELKSINIKELISYSEN |
| 49 | Capsule_ | P19579 | MMKRPSIARVVKVIICLLTPILLSFSGIGDNDIDKK |
| | biosynthesis_ | | KSTSKEVDDTLRIVITGDLLLDRGVRQKIDMAGV |
| | protein_CapA | | DALFSPTIDSLFHSSNYVIANLECPVTKIRERVFKR |
| | | | FIFRGEPEWLPTLRRHGITHLNLANNHSIDQGRNG |
| | | | LLDTQEQIKKAGMIPIGAGKNMEEAAEPVLISTSP |
| | | | RHVWVISSLRLPLENFLYLPQKPCVSQESIDSLIMR |
| | | | VKRLRATDKNCYILLILHWGWEHHFRATPQQRED |
| | | | AHKLIDAGADAIVGHHSHTLQTIETYRGKPIYYGI |
| | | | GNFIFDQRKPMNSRACLVELSITAEKCKAKALPIEI |
| | | | KNCTPYLSK |
| 50 | Peptidoglycan_ | B5ZA76 | MILLSFDTEEFDVPREHGVDFSLEEGMKVSIEGTN |
| | deacetylase | | RILDILKANNVCATFFCTGNFAELAPEVMERIKNE |
| | | | GHEVACHGVDHWQPKPEDVFRSKEIIERVTGVKV |
| | | | AGYRQPRMFPVSDEDIEKAGYLYNSSLNPAFIPGR |
| | | | YMHLTTSRTWFMQGKVMQIPASVSPHLRIPLFWL |
| | | | SMHNFPEWFYLRLVRQVLRHDGYFVTYFHPWEF |
| | | | YDLKSHPEFKMPFIIKNHSGHELEQRLDRFIKAMK |
| | | | ADKQEFITYVDFVNRQKK |
| 51 | Fumarate_reductase_ | P0AC47 | MAKNISFTIKYWKQNGPQDQGHFDTHEMKNIPD |
| | iron- | | DTSFLEMLDILNEELIAAGDEPFVFDHDCREGICG |
| | sulfur_subunit | | MCSLYINGTPHGKTERGATTCQLYMRRFNDGDVI |
| | | | TVEPWRSAGFPVIKDCMVDRTAFDKIIQAGGYTTI |
| | | | RTGQAQDANAILISKDNADEAMDCATCIGCGACV |
| | | | AACKNGSAMLFVSSKVSQLALLPQGKPEAAKRA |
| | | | KAMVAKMDEVGFGNCTNTRACEAVCPKNEKIAN |
| | | | IARLNREFIKAKFAD |
| 52 | Serine/threonine- | P9WI71 | MSENKLSTNEQAQTADAPVKASYTEYKVIPSQGY |
| | protein_kinase_ | | CMIVKCRKGDQTVVLKTLKEEYRERVLLRNALK |
| | PknH | | REFKQCQRLNHSGIVRYQGLVEVDGYGLCIEEEY |
| | | | VEGRTLQAYLKENHTDDEKIAIINQIADALRYAHQ |
| | | | QGVIHRNLKPSNVLVTTQGDYVKLIDFSVLSPEDV |
| | | | KPTAETTRFMAPEMKDETLTADATADIYSLGTIM |
| | | | KVMGLTLAYSEVIKRCCAFKRSDRYSNVDELLAD |
| | | | LNNEGSSFSMPKIGKGTVVLGLIIAVVIGIGALLYN |
| | | | YGGALIDQVGKIDVSSVFSSDAETAPEDTVKVNT |
| | | | AEQSDSLSTEAEAPAIGKLAFMNRMKPALYKDLD |
| | | | NIFEKNSADKAKLTKAIKTYYRGLIQANDTLDNE |
| | | | QRAEVDRVFGDYVKQKKAALN |
| 53 | Carboxy- | O34666 | MRKYICLLLFYLFTFLPLSAQQGNDSPLRKLQLAE |
| | terminal_processing_ | | MAIKNFYVDSVNEQKLVEDGIRGMLEKLDPHSTY |
| | protease_CtpA | | TDAKETKAMNEPLQGDFEGIGVQFNMIEDTLVVI |
| | | | QPVVNGPSQKVGILAGDRIVSVNDSTIAGVKMARI |
| | | | DIMKMLRGKKGTKVKLGVVRRGVKGVLTFVVTR |
| | | | AKIPVHTINASYMIRPNVGYIRIESFGMKTHDEFM |
| | | | SAVDSLKKKGMKTLLLDLQDNGGGYLQSAVQIS |
| | | | NEFLKNNDMIVYTEGRRARRQNFKAIGNGRLQD |
| | | | VKVYVLVNELSASAAEIVTGAIQDNDRGTVVGRR |
| | | | TFGKGLVQRPFDLPDGSMIRLTIAHYYTPSGRCIQ |
| | | | KPYTKGDLKDYEMDIEKRFKHGELTNPDSIQFSDS |
| | | | LKYYTIRKHRVVYGGGGIMPDNFVPLDTTKFTRY |
| | | | HRMLAAKSIIINAYLKYADANRQALKAQYSSFDA |
| | | | FNKGYVVPQSLLDEIVAEGKKEKIEPKDAAELKA |
| | | | TLPNIALQIKALTARDIWDMNEYFRVWNTQSDIV |
| | | | NKAVALATGK |
| 54 | Cluster: | D9RRG3 | MKLTEQRSSMLHGVLLITLFACAAFYIGDMGWV |
| | Uncharacterized | | KALSLSPMVVGIILGMLYANSLRNNLPDTWVPGI |
| | protein | | AFCGKRVLRFGIILYGFRLTFQDVVAVGFPAIIVD |
| | | | AIIVSGTILLGVLVGRLLKMDRSIALLTACGSGICG |
| | | | AAAVLGVDGAIRPKPYKTAVAVATVVIFGTLSMF |
| | | | LYPILYRAGIFDLSPDAMGIFAGSTIHEVAHVVGA |
| | | | GNAMGAAVSNSAIIVKMIRVMMLVPVLLVIAFFV |
| | | | AKNVAERDDEAGGSRKINIPWFAILFLVVIGENSL |
| | | | NLLPKELVDFINTLDTFLLTMAMSALGAETSIDKF |
| | | | KKAGFKPFLLAAILWCWLIGGGYCLAKYLVPVLG |
| | | | VAC |
| 55 | Cluster: Cna | X6Q2J4 | MNKQFLLAALWLSPLGLYAHKANGIGAVTWKNE |
| | protein B-type | | APKERMIRGIDEDKTHQRFTLSGYVKDRNGEPLIN |
| | domain protein | | ATIYDLTTRQGTMTNAYGHFSLTLGEGQHEIRCS |
| | | | YVGYKTLIETIDLSANQNHDIILQNEAQLDEVVVT |
| | | | TDLNSPLLKTQTGKLSLSQKDIKTEYALLSSPDVIK |
| | | | TLQRTSGVADGMELASGLYVHGGNGDENLFLLD |
| | | | GTPLYHTNHSLGLFSSFNADVVKNVDFYKSGFPA |
| | | | RYGGRLSSVIDVRTADGDLYKTHGSYRIGLLDGA |
| | | | FHIGGPIRKGKTSYNFGLRRSWMDLLTRPAFAIMN |
| | | | HKSDNEDKLSMSYFFHDLNFKLTNIFNERSRMSLS |
| | | | VYSGEDRLDAKDEWHSNNSSGYNDVDIYVNRFH |
| | | | WGNFNAALDWNYQFSPKLFANFTAVYTHNRSTV |
| | | | SSSDEWRFTRPGEKEQLTLTSHGYRSSIDDIGYRA |
| | | | AFDFRPSPRHHIRFGQDYTYHRFQPQTYNRFDNY |
| | | | QTNSEAKADTIATHSYNKNVAHQLTFYAEDEMTL |
| | | | NEKWSLNGGVNADVFHISGKTFATLSPRLSMKFQ |
| | | | PTERLSLKASYTLMSQFVHKIANSFLDLPTDYWVP |
| | | | TTARLHPMRSWQVAAGAYMKPNKHWLLSLEAY |
| | | | YKRSSHILQYSSWAGLEPPAANWDYMVMEGDGR |
| | | | SYGVELDADYNVSNLTLHGSYTLSWTQKKFDDF |
| | | | YDGWYYDKFDNRHKLTLTGRWNITKKIAAFAAW |
| | | | TFRTGNRMTIPTQYIGLPDVPAQEQGGLTFNSSDD |
| | | | NTLNFAYEKPNNVILPAYHRLDIGFDFHHTTKKG |
| | | | HERIWNLSFYNAYCHLNSLWVRVKIDSNNQMKIR |
| | | | NIAFIPVIPSFSYTFKF |
| 56 | Poly-beta-1,6-N- | P75905 | MSKQVFQTDSRQRWSYFKWTLRVILTILSLLGIVE |
| | acetyl-D- | | LAMFALEGSPQMPFRHDYRNAVTAASPYTKDNK |
| | glucosamine_ | | TAKLYKSFRDFFKEKKMHNNYAKATIKKQRFIGK |
| | synthase | | ADSVTQKYFREWDDPRIGVRSAWYVNWDKHAYI |
| | | | SLKNNIKHLNMVLPEWFFINPKTDKVEYRIDKQA |
| | | | LRLMRRTGIPVLPMLTNNYNSDFHPEAIGRIMRDE |
| | | | KKRMALINEMVRTCRHYGFAGINLDLEELNIQDN |
| | | | DLLVELLKDFSRVFHANGLYVTQAVAPFNEDYN |
| | | | MQELAKYNDYLFLMAYDEHNIESQPGAVSSQRW |
| | | | VEKATDWAAKNVPNDKIVLGMATYGYDWANGE |
| | | | GGTTVSFDQTMAIAQDADAKVKFDDDTYNVNFS |
| | | | YQNTDDGKIHHVFFTDAATTFNIMRFGAEYHLAG |
| | | | YGLWRLGTEDKRIWRFYGKDMSWENVARMSVA |
| | | | KLMQLNGTDDVNFVGSGEVLEVTTEPHPGDISIRI |
| | | | DKDNRLISEEYYRALPSTYTIQRLGKCKDKQLVIT |
| | | | FDDGPDSRWTPTVLSTLKKYNVPAAFFMVGLQM |
| | | | EKNLPLVKQVYEDGHTIGNHTFTHHNMIENSDRR |
| | | | SYAELKLTRMLIESVTGHSTILFRAPYNADADPTE |
| | | | HEEIWPMIVASRRNYLFVGESIDPNDWEPNVTSD |
| | | | QIYQRVIDGVHHEDGHIILLHDAGGSSRKPTLDAL |
| | | | PRIIETLQHEGYQFISLEQYLGMGKQTLMPEINKG |
| | | | KAYYAMQTNLWLAEMIYHVSDFLTALFLVFLAL |
| | | | GMMRLIFMYVLMIREKRAENRRNYAPIDAATAPA |
| | | | VSIIVPGYNEEVNIVRTITTLKQQDYPNLHIYFVDD |
| | | | GSKDHTLERVHEAFDNDDTVTILAKKNGGKASAL |
| | | | NYGIAACRSEYVVCIDADTQLKNDAVSRLMKHFI |
| | | | ADTEKRVGAVAGNVKVGNQRNMLTYWQAIEYT |
| | | | SSQNFDRMAYSNINAITVVPGAIGAFRKEVIEAVG |
| | | | GFTTDTLAEDCDLTMSINEHGYIIENENYAVALTE |
| | | | APETLRQFVKQRIRWCFGVMQAFWKHRSSLFAPS |
| | | | KKGFGLWAMPNMLIFQYIIPTFSPLADVLMLIGLF |
| | | | TGNALQIFFYYLIFLVIDASVSIMAYIFEGERLWVL |
| | | | LWVIPQRFFYRWIMYYVLFKSYLKAIKGELQTWG |
| | | | VLKRTGHVKG |
| 57 | Cell_division_ | O34876 | MAKKRNKARSRHSLQVVTLCISTAMVLMLIGIVV |
| | protein_FtsX | | LTGFTSRNLSSYVKENLTITMILQPDMNTEESAAL |
| | | | CERIRTLHYINSLNFISKEQALKDGTKELGANPAEF |
| | | | AGENPFTGEIEVQLKANYANNDSIRNIVQQLRTYR |
| | | | GVSDITYPQSLVESVNQTLGKISLVLLVIAVLLTIIS |
| | | | FSLINNTIRLSIYAHRFSIHTMKLVGGSWSFIRAPFL |
| | | | RRAVLEGLVSALLAIAVLGIGICLLYEKEPEITKLL |
| | | | SWDALIITAIVMLAFGVIIATFCAWLSVNKFLRMK |
| | | | AGDLYKI |
| 58 | UDP-2,3- | P44046 | MKNIYFLSDAHLGSLAIDHRRTHERRLVRFLDSIK |
| | diacylglucosamine_ | | HKAAAVYLLGDMFDFWNEYKYVVPKGFTRFLG |
| | hydrolase | | KISELTDMGVEVHFFTGNHDLWTYGYLEKECGVI |
| | | | LHRKPITTEIYDKVFYLAHGDGLGDPDPMFRFLRK |
| | | | VFHNRFCQRLLNFFHPWWGMQLGLNWAKRSRL |
| | | | KRKDGKEVPYLGEDKEYLVQYTKEYMSTHKDID |
| | | | YYIYGHRHIELDLTLSRKARLLILGDWIWQFTYAV |
| | | | FDGEHMFLEEYVEGESKP |
| 59 | Poly-beta-1,6-N- | P75905 | MVGLDVLCYFIHAKGREKECYFERIIYQITCHSRT |
| | acetyl-D- | | KCYLCNIMKYSIIVPVFNRPDEVEELLESLLSQEEK |
| | glucosamine_ | | DFEVVIVEDGSQIPCKEVCDKYADKLDLHYYSKE |
| | synthase | | NSGPGQSRNYGAERAKGEYLLILDSDVVLPKGYI |
| | | | CAVSEELKREPADAFGGPDCAHESFTDTQKAISYS |
| | | | MTSFFTTGGIRGGKKKLDKFYPRSFNMGIRRDVY |
| | | | QELGGFSKMRFGEDIDFSIRIFKAGKRCRLFPEAW |
| | | | VWHKRRTDFRKFWKQVYNSGIARINLYKKYPESL |
| | | | KLVHLLPMVFTVGTALLVLMILFGLFLQLFPIINVF |
| | | | GSVFIMMGLMPLVLYSVIICVDSTMQNNSLNIGLL |
| | | | SIEAAFIQLTGYGCGFISAWWKRCVCGMDEFAAY |
| | | | EKNFYK |
| 60 | Enolase | Q8DTS9 | MKIEKVHAREIMDSRGNPTVEVEVTLENGVMGR |
| | | | ASVPSGASTGENEALELRDGDKNRFLGKGVLKAV |
| | | | ENVNNLIAPALKGDCVLNQRAIDYKMLELDGTPT |
| | | | KSKLGANAILGVSLAVAQAAAKALNIPLYRYIGG |
| | | | ANTYVLPVPMMNIINGGAHSDAPIAFQEFMIRPVG |
| | | | APSEKEGIRMGAEVFHALAKLLKKRGLSTAVGDE |
| | | | GGFAPKFDGIEDALDSIIQAIKDAGYEPGKDVKIA |
| | | | MDCAASEFAVCEDGKWFYDYRQLKNGMPKDPN |
| | | | GKKLSADEQIAYLEHLITKYPIDSIEDGLDENDWE |
| | | | NWVKLTSAIGDRCQLVGDDLFVTNVKFLEKGIK |
| | | | MGAANSILIKVNQIGSLTETLEAIEMAHRHGYTTV |
| | | | TSHRSGETEDTTIADIAVATNSGQIKTGSMSRTDR |
| | | | MAKYNQLIRIEEELGACAKYGYAKLK |
| 61 | Outer_membrane_ | Q8G0Y6 | MKKLFTIAMLLGVTLGIHAQEVYSLQKCRELALQ |
| | efflux_protein_ | | NNRQLKVSRMTVDVAENTRKAAKTKYLPRVDAL |
| | BepC | | AGYQHFSREISLLSDDQKNAFSNLGTNTFGQLGG |
| | | | QIGQNLTSLAQQGILSPQMAQQLGQLFSNVATPLT |
| | | | QVGNNIGQSINDAFRSNTKNVYAGGIVVNQPIYM |
| | | | GGAIKAANDMAAIGEQVAQNNISLKRQLVLYGV |
| | | | DNAYWLAISLKKKEALAIRYRDLAQKLNEDVKK |
| | | | MIREGVATRADGLKVEVAVNTADMQIARIQSGVS |
| | | | LAKMALCELCGLELNGDIPLSDEGDADLPPTPSTQ |
| | | | FDNYTVSSSDTTGLNEARPELRLLQNAVDLSIQNT |
| | | | KLIRSLYMPHVLLTAGYSVSNPNLFNGFQKRFTDL |
| | | | WNIGITVQVPVWNWGENKYKVRASKTATTIAQL |
| | | | EMDDVRKKIDLEIEQNRLRLKDANKQLATSQKN |
| | | | MAAAEENLRCANVGFKEGVMTVTEVMAAQTAW |
| | | | QTSRMAIIDAEISVKLAQTGLQKALGGL |
| 62 | Phosphoethanolamine_ | Q7CPC0 | MKRTFVTKMVKPIEENSLFFMFMLLVGAFTNVSH |
| | transferase_ | | RNVFGYIELIADVYIICFLLSLCQRTIRQGLVIMLSS |
| | CptA | | VIYVVAIIDTCCKTLFDTPITPTMLLLAQETTGREA |
| | | | TEFFLQYLNLKLFFSAADIILFLAFCHIVMAVKKM |
| | | | KFSTSYLKQPFVAFVLMFTIFVGMALSIYDKVQLY |
| | | | TVKNLSGLEVAVINGFAHLYHPVERIVYGLYSNH |
| | | | LIAKQVDGVIMANQQIKVDSCSFTSPTIVLVIGESA |
| | | | NRHHSQLYGYPLPTTPYQLAMKNGKDSLAVFTN |
| | | | VVSPWNLTSKVFKQIFSLQSVDEKGDWSKYVLFP |
| | | | AVFKKAGYHVSFLSNQFPYGINYTPDWTNNLVG |
| | | | GFFLNHPQLNKQMFDYRNVTIHNYDEDLLNDYK |
| | | | EIISYKKPQLIIFHLLGQHFQYSLRCKSNMKKFGIK |
| | | | DYKRMDLTDKEKQTIADYDNATLYNDFVLNKIV |
| | | | EQFRNKDAIIVYLSDHGEDCYGKDVNMAGRLTE |
| | | | VEQINLKKYHEEFEIPFWIWCSPIYKQRHRKIFTET |
| | | | LMARNNKFMTDDLPHLLLYLAGIKTKDYCEERN |
| | | | VISPSFNNNRRRLVLKTIDYDKALYQ |
| 63 | Dipeptide_and_ | P36837 | MFKNHPKGLLQAAFSNMGERFGYYIMNAVLALF |
| | tripeptide_permease_ | | LCSKFGLSDETSGLIASLFLAAIYVMSLVGGVIAD |
| | B | | RTQNYQRTIESGLVVMALGYVALSIPVLATPENNS |
| | | | YLLAFTIFALVLIAVGNGLFKGNLQAIVGQMYDD |
| | | | FETEAAKVSPERLKWAQGQRDAGFQIFYVFINLG |
| | | | ALAAPFIAPVLRSWWLGRNGLTYDAALPQLCHK |
| | | | YINGTIGDNLGNLQELATKVGGNSADLASFCPHY |
| | | | LDVFNTGVHYSFIASVVTMLISLIIFMSSKKLFPMP |
| | | | GKKEQIVNVEYTDEEKASMAKEIKQRMYALFAV |
| | | | LGISVFFWFSFHQNGQSLSFFARDFVNTDSVAPEI |
| | | | WQAVNPFFVISLTPLIMWVFAYFTKKGKPISTPRK |
| | | | IAYGMGIAGFAYLFLMGFSLVHNYPSAEQFTSLEP |
| | | | AVRATMKAGPMILILTYFFLTVAELFISPLGLSFVS |
| | | | KVAPKNLQGLCQGLWLGATAVGNGFLWIGPLMY |
| | | | NKWSIWTCWLVFAIVCFISMVVMFGMVKWLERV |
| | | | TKS |
| 64 | C4- | Q914F5 | MQKKIKIGLLPRVIIAILLGLFLGYYLPDPAVRVFL |
| | dicarboxylate_ | | TFNSIFSQFLGFMIPLIIIGLVTPAIAGIGKGAGKLLL |
| | transport_protein_2 | | ATVAIAYVDTIVAGGLSYGTGTWLFPSMIASTGG |
| | | | AIPHIDKATELTPYFTINIPAMVDVMSSLVFSFIAG |
| | | | LGIAYGGLRTMENLFNEFKTVIEKVIEKAIIPLLPL |
| | | | YIFGVFLSMTHNGQARQVLLVFSQIIIVILVLHVLI |
| | | | LIYEFCIAGAIVKHNPFRLLWNMLPAYLTALGTSS |
| | | | SAATIPVTLKQTVKNGVSEEVAGFVVPLCATIHLS |
| | | | GSAMKITACALTICMLTDLPHDPGLFIYFILMLAII |
| | | | MVAAPGVPGGAIMAALAPLSSILGFNEEAQALMI |
| | | | ALYIAMDSFGTACNVTGDGAIALAVNKFFGKKKE |
| | | | TSILS |
| 65 | Inner_membrane_ | P76090 | MISVYSIKPQFQRVLTPILELLHRAKVTANQITLW |
| | protein_YnbA | | ACVLSLVIGILFWFAGDVGTWLYLCLPVGLLIRM |
| | | | ALNALDGMMARRYNQITRKGELLNEVGDVVSDT |
| | | | IIYFPLLKYHPESLYFIVAFIALSIINEYAGVMGKVL |
| | | | SAERRYDGPMGKSDRAFVLGLYGVVCLFGINLSG |
| | | | YSVYIFGVIDLLLVLSTWIRIKKTLKVTRNSQTPE |
| 66 | 2′,3′-cyclic- | P08331 | MKLSTILLSIMLGLSSSTMAQQKDVTIKLIETTDV |
| | nucleotide | | HGSFFPYDFITRKPKSGSMARVYTLVEELRKKDG |
| | | | KDNVYLLDNGDILQGQPISYYYNYVAPEKTNIAA |
| | | | SVLNYMGYDVATVGNHDIETGHKVYDKWFKEL |
| | | | KFPILGANIIDTKTNKPYILPYYTIKKKNGIKVCVIG |
| | | | MLTPAIPNWLKESIWSGLRFEEMVSCAKRTMAEV |
| | | | KTQEKPDVIVGLFHSGWDGGIKTPEYDEDASKKV |
| | | | AKEVPGFDIVFFGHDHTPHSSIEKNIVGKDVICLDP |
| | | | ANNAQRVAIATLTLRPKTVKGKRQYTVTKATGEL |
| | | | VDVKELKADDAFIQHFQPEIDAVKAWSDQVIGRF |
| | | | ENTIYSKDSYFGNSAFNDLILNLELEITKADIAFNA |
| | | | PLLFNASIKAGPITVADMFNLYKYENNLCTMRLT |
| | | | GKEIRKHLEMSYDLWCNTMKSPEDHLLLLSSTQN |
| | | | DAQRLGFKNFSFNFDSAAGIDYEVDVTKPDGQKV |
| | | | RILRMSNGEPFDENKWYTVAVNSYRANGGGELL |
| | | | TKGAGIPRDSLKSRIIWESPKDQRHYLMEEIKKAG |
| | | | VMNPQPNHNWKFIPETWTVPAAARDRKLLFGE |
| 67 | Fe(2+)_transporter_ | P33650 | MKLSELKTGETGVIVKVSGHGGFRKRIIEMGFIKG |
| | FeoB | | KTVEVLLNAPLQDPVKYKIMGYEVSLRHSEADQI |
| | | | EVLSDVKTHSVGNEEEQEDNQLEMDSTTYDSTDK |
| | | | ELTPEKQSDAVRRKNHTINVALVGNPNCGKTSLF |
| | | | NFASGAHERVGNYSGVTVDAKVGRAEFDGYVEN |
| | | | LVDLPGTYSLSAYSPEELYVRKQLVDKTPDVVIN |
| | | | VIDSSNLERNLYLTTQLIDMHIRMVCALNMFDETE |
| | | | QRGDHIDAQKLSELFGVPMIPTVFTNGRGVKELFR |
| | | | QIIAVYEGKEDESLQFRHIHINHGHEIENGIKEMQE |
| | | | HLKKYPELCHRYSTRYLAIKLLEHDKDVEQLVSP |
| | | | LGDSIEIFNHRDTAAARVKEETGNDSETAIMDAK |
| | | | YGFINGALKEANFSTGDKKDTYQTTHVIDHVLTN |
| | | | KYFGFPIFFLVLLVMFTATFVIGQYPMDWIEAGVG |
| | | | WLGEFISKNMPAGPVKDMIVDGIIGGVGAVIVFLP |
| | | | QILILYFFISYMEDCGYMSRAAFIMDRLMHKMGL |
| | | | HGKSFIPLIMGFGCNVPAVMATRTIESRRSRLITML |
| | | | ILPLMSCSARLPIYVMITGSFFALKYRSLAMLSLYII |
| | | | GVLMAVAMSRLFSAFVVKGEDTPFVMELPPYRFP |
| | | | TWKAIGRHTWEKGKQYLKKMGGIILVASIIVWAL |
| | | | GYFPLPDDPNMDNQARQEQSYIGRIGKAVEPVFR |
| | | | PQGFNWKLDVGLLSGMGAKEIVASTMGVLYSND |
| | | | GSFSDDNGYSSETGKYSKLHNLITKDVATMHHIS |
| | | | YEEAEPIATLTAFSFLLFVLLYFPCVATIAAIKGET |
| | | | GSWGWALFAAGYTTALAWIVSAVVFQVGMLFM |
| 68 | UDP-N- | P9WJM1 | MESFIIEGGHQLSGTIAPQGAKNEALEVICATLLTS |
| | acetylglucosamine | | EEVIIRNVPDILDVNNLIKLLQDIGVKVKKLAPNEF |
| | | | SFQADEVNLDYLESSDFVKKCSSLRGSVLMIGPLL |
| | | | GRFGKATIAKPGGDKIGRRRLDTHFLGFKNLGAH |
| | | | FGRVEDRDVYEIQADKLVGTYMLLDEASITGTAN |
| | | | IIMAAVLAEGTTTIYNAACEPYIQQLCKMLNAMG |
| | | | AKISGIASNLITIEGVKELHSADHRILPDMIEVGSFI |
| | | | GIAAMIGDGVRIKDVSVPNLGLILDTFHRLGVQIIV |
| | | | DNDDLIIPRQDHYVIDSFIDGTIMTISDAPWPGLTP |
| | | | DLISVLLVVATQAQGSVLFHQKMFESRLFFVDKLI |
| | | | DMGAQIILCDPHRAVVVGHDNAKKLRAGRMSSP |
| | | | DIRAGIALLIAALTAQGTSRIDNIVQIDRGYENIEG |
| | | | RLNALGAKIQRAEVC |
| 69 | Ribitol-5- | Q8RKI9 | MNIAVIFAGGSGLRMHTKSRPKQFLDLNGKPIIIYT |
| | phosphate_ | | LELFDNHPNIDAIVVACIESWIPFLEKQLRKFEINK |
| | cytidylyltransferase | | VVKIIPGGKSGQESIYKGLCAAEEYAQSKGVSNEE |
| | | | TTVLIHDGVRPLITEETITDNIKKVEEVGSCITCIPA |
| | | | TETLIVKQADDALEIPSRADSFIARAPQSFRLIDIIT |
| | | | AHRRSLAEGKADFIDSCTMMSHYGYKLGTIIGPM |
| | | | ENIKITTPTDFFVLRAMVKVHEDQQIFGL |

In some embodiments, the Prevotella bacteria are from a strain of Prevotella bacteria comprising one or more of the proteins listed in Table 1 and that is free or substantially free of one or more proteins listed in Table 2. In some embodiments, the Prevotella bacteria are from a strain of Prevotella bacteria that comprises all of the proteins listed in Table 1 and/or all of the genes encoding the proteins listed in Table 1 and that is free of all of the proteins listed in Table 2 and/or all of the genes encoding the proteins listed in Table 2.

In some embodiments, the engineered Prevotella bacteria described herein are modified to improve Prevotella bacterial (e.g., higher oxygen tolerance, stability, improved freeze-thaw tolerance, shorter generation times). For example, in some embodiments, the engineered Prevotella bacteria described include bacteria harboring one or more genetic changes, such change being an insertion, deletion, translocation, or substitution, or any combination thereof, of one or more nucleotides contained on the bacterial chromosome or endogenous plasmid and/or one or more foreign plasmids, wherein the genetic change may results in the overexpression and/or underexpression of one or more genes. The engineered microbe(s) may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, or any combination thereof.

In some embodiments, the Prevotella bacteria described herein are modified such that they comprise, are linked to, and/or are bound by a therapeutic moiety.

### Bacterial Compositions

In certain embodiments, the methods provided herein comprise use of bacterial compositions (e.g., pharmaceutical compositions) comprising Prevotella bacteria provided herein.

In some embodiments, the bacterial compositions comprise whole Prevotella histicola bacteria (e.g., live bacteria, killed bacteria, and/or attenuated bacteria). In some embodiments, the Prevotella histicola bacteria are non-viable. In some embodiments, the Prevotella histicola bacteria has been gamma irradiated (e.g., according to a method described herein). In some embodiments, the Prevotella histicola bacteria are live.

In some embodiments, the bacterial composition (e.g., pharmaceutical composition) comprises only one strain of bacteria, e.g., Prevotella histicola.

In some embodiments, the Prevotella histicola is Prevotella Strain B 50329 (NRRL accession number B 50329). In some embodiments, the Prevotella strain is a strain comprising at least at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain B 50329.

In some embodiments, the bacterial compositions comprise whole Prevotella histicola bacteria (e.g., live bacteria, killed bacteria, attenuated bacteria). In some embodiments, the Prevotella histicola bacteria are non-viable.

In some embodiments, the bacterial composition comprises about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², and/or 1×10¹³ total cells of Prevotella histicola.

In some embodiments, the bacterial composition comprises at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 0.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², or 1×10¹³ total cells of Prevotella histicola.

In some embodiments, the bacterial composition comprises at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10′⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², or 1×10¹³ total cells of Prevotella histicola.

In some embodiments, the bacterial composition comprises from about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ to about 8×10¹¹ total cells of Prevotella histicola.

In some embodiments, the bacterial composition comprises from about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹, 2.7×10¹¹, 2.8×10¹¹, 2.9×10¹¹, 3×10¹, 3.1×10¹¹, 3.2×10¹¹, 3.3×10¹¹, 3.4×10¹¹, 3.5×10¹, 3.6×10¹¹, 3.7×10¹¹, 3.8×10¹¹, 3.9×10¹¹, 4×10¹¹ to about 1×10¹² total cells of Prevotella histicola.

In some embodiments, the bacterial composition comprises about 1.6×10¹⁰ total cells of Prevotella histicola, e.g., of Prevotella Strain B 50329.

In some embodiments, the bacterial composition comprises about 8×10¹⁰ total cells of Prevotella histicola, e.g., of Prevotella Strain B 50329.

In some embodiments, the bacterial composition comprises about 1.6×10¹¹ total cells of Prevotella histicola, e.g., of Prevotella Strain B 50329.

In some embodiments, the bacterial composition comprises about 3.2×10¹¹ total cells of Prevotella histicola, e.g., of Prevotella Strain B 50329.

In some embodiments, the bacterial composition comprises about 8×10¹¹ total cells of Prevotella histicola, e.g., of Prevotella Strain B 50329.

In some embodiments, the bacterial composition comprises about 1.6×10¹⁰ to about 8×10¹¹ total cells of Prevotella histicola, e.g., of Prevotella Strain B 50329.

In some embodiments, the bacterial composition comprises about 1.6×10¹⁰ to about 1.6×10¹¹ total cells of Prevotella histicola, e.g., of Prevotella Strain B 50329.

In some embodiments, the bacterial composition comprises about 1.6×10¹¹ to about 8×10¹¹ total cells of Prevotella histicola, e.g., of Prevotella Strain B 50329.

In some embodiments, the bacterial composition comprises about 8×10¹⁰ to about 8×10¹¹ total cells of Prevotella histicola, e.g., of Prevotella Strain B 50329.

In some embodiments, the Prevotella bacteria may be quantified based on total cells, e.g., total cell count (TCC) (e.g., determined by Coulter counter).

In some embodiments, the bacterial composition comprises at least 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola.

In some embodiments, the bacterial composition comprises at most 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola.

In some embodiments, the bacterial composition comprises about 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola.

In some embodiments, the bacterial composition is administered orally. In some embodiments, the administration to the subject once daily. In some embodiments, the bacterial composition is administered in 2 or more doses (e.g., 3 or more, 4 or more or 5 or more doses). In some embodiments, the administration to the subject of the two or more doses are separated by at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days.

In some embodiments, the bacterial composition is administered once daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

In some embodiments, the bacterial composition is administered once daily for 1, 2, 3, 4, 5, 6, 7, 8, 9, 10, 11, 12, 13, 14, 15, 16, or 20 weeks. In some embodiments, the bacterial composition is administered once daily for at least 1, 2, 3, 4, 5, 6, 7, 8, 9, 10, 11, 12, 13, 14, 15, 16, or 20 weeks.

In some embodiments, the bacterial composition is formulated as a capsule or a tablet. In some embodiments, the bacterial formulation (e.g., composition) comprises an enteric coating or micro encapsulation. In some embodiments, the capsule is an enteric coated capsule (e.g., HPMC coated). In some embodiments, the enteric coating allows release of the bacterial composition in the small intestine, e.g., in the upper small intestine, e.g., in the duodenum. In some embodiments, the enteric coating comprises HPMC.

In some embodiments, the subject is a mammal. In some embodiments, the subject is a human. In some embodiments, the subject is a non-human mammal (e.g., a dog, a cat, a cow, a horse, a pig, a donkey, a goat, a camel, a mouse, a rat, a guinea pig, a sheep, a llama, a monkey, a gorilla, or a chimpanzee).

In some embodiments, to quantify the numbers of Prevotella histicola bacteria present in a bacterial sample, electron microscopy (e.g., EM of ultrathin frozen sections) can be used to visualize the bacteria and count their relative numbers. Alternatively, combinations of nanoparticle tracking analysis (NTA), Coulter counting, and dynamic light scattering (DLS) or a combination of these techniques can be used. NTA and the Coulter counter count particles and show their sizes. DLS gives the size distribution of particles, but not the concentration. Bacteria frequently have diameters of 1-2 μm. The full range is 0.2-20 μm. Combined results from Coulter counting and NTA can reveal the numbers of bacteria in a given sample. Coulter counting reveals the numbers of particles with diameters of 0.7-10 m. NTA reveals the numbers of particles with diameters of 50-1400 nm. For most bacterial samples, the Coulter counter alone can reveal the number of bacteria in a sample.

In some embodiments, the bacterial composition comprises an enteric coating or micro encapsulation. In certain embodiments, the enteric coating or micro encapsulation improves targeting to a desired region of the gastrointestinal tract. For example, in certain embodiments, the bacterial composition comprises an enteric coating and/or microcapsules that dissolves at a pH associated with a particular region of the gastrointestinal tract. In some embodiments, the enteric coating and/or microcapsules dissolve at a pH of about 5.5-6.2 to release in the duodenum, at a pH value of about 7.2-7.5 to release in the ileum, and/or at a pH value of about 5.6-6.2 to release in the colon. Exemplary enteric coatings and microcapsules are described, for example, in U.S. Pat. Pub. No. 2016/0022592, which is hereby incorporated by reference in its entirety. In some embodiments, the enteric coating comprises HPMC.

In certain aspects, provided are bacterial compositions for administration subjects. In some embodiments, the bacterial compositions are combined with additional active and/or inactive materials in order to produce a final product, which may be in single dosage unit or in a multi-dose format. In some embodiments, the bacterial compositions is combined with an adjuvant such as an immuno-adjuvant (e.g., STING agonists, TLR agonists, NOD agonists).

In some embodiments the composition comprises at least one carbohydrate. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

In some embodiments the composition comprises at least one lipid. As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some embodiments the composition comprises at least one modified lipid, for example a lipid that has been modified by cooking.

In some embodiments the composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments the composition comprises at least one supplemental vitamin. The at least one vitamin can be fat-soluble or water-soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments the composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments the excipient is a buffering agent. Non-limiting examples of suitable buffering agents include sodium citrate, magnesium carbonate, magnesium bicarbonate, calcium carbonate, and calcium bicarbonate.

In some embodiments the excipient comprises a preservative. Non-limiting examples of suitable preservatives include antioxidants, such as alpha-tocopherol and ascorbate, and antimicrobials, such as parabens, chlorobutanol, and phenol.

In some embodiments the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments the composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments the composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments the composition is a food product for animals, including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto.

### Dose Forms

Dose forms comprising Prevotella histicola bacteria are also provided herein, e.g., for use in methods to treat or prevent inflammation (such as Th1-, Th2-, or Th17-mediated inflammation) in a subject (e.g., a human subject). Dose forms comprising Prevotella histicola bacteria are also provided herein, e.g., for use in methods to treat or prevent atopic dermatitis and/or psoriasis in a subject (e.g., a human subject). A bacterial composition (e.g., pharmaceutical composition) comprising Prevotella histicola bacteria can be formulated as a solid dose form, e.g., for oral administration. The solid dose form can comprise one or more excipients, e.g., pharmaceutically acceptable excipients. The Prevotella histicola bacteria in the solid dose form can be isolated Prevotella histicola bacteria. Optionally, the Prevotella histicola bacteria in the solid dose form can be lyophilized. Optionally, the Prevotella histicola bacteria in the solid dose form are live. Optionally, the Prevotella histicola bacteria in the solid dose form are non-viable. Optionally, the Prevotella histicola bacteria in the solid dose form are gamma irradiated. The solid dose form can comprise a tablet, a minitablet, a capsule, a pill, or a powder; or a combination of these forms (e.g., minitablets comprised in a capsule).

The Prevotella histicola bacteria in the solid dose form can be in a powder (e.g., the powder comprises lyophilized Prevotella histicola bacteria). In some embodiments, the powder further comprises mannitol, magnesium stearate, and/or colloidal silicon dioxide. In some embodiments, the powder further comprises mannitol, magnesium stearate, and colloidal silicon dioxide.

In some embodiments, the lyophilized Prevotella bacteria are resuspended in a solution.

In certain embodiments, the bacterial composition (e.g., pharmaceutical composition) provided herein is prepared as a solid dosage form comprising Prevotella histicola bacteria and a pharmaceutically acceptable carrier.

In some embodiments, the solid dosage form comprises a capsule. The capsule can comprise an enteric coating. The capsule can be a size 00, size 0, size 1, size 2, size 3, size 4, or size 5 capsule. The capsule can comprise Prevotella histicola bacteria powder (e.g., lyophilized Prevotella histicola bacteria). In some embodiments, the powder further comprises mannitol, magnesium stearate, and/or colloidal silicon dioxide. In some embodiments, the powder further comprises mannitol, magnesium stearate, and colloidal silicon dioxide.

In some embodiments, the solid dosage form described herein can be, e.g., a tablet or a mini-tablet. In some embodiments, a plurality of mini-tablets can be in (e.g., loaded into) a capsule.

In some embodiments, the solid dosage form comprises a tablet (>4 mm) (e.g., 5 mm-17 mm). For example, the tablet is a 5 mm, 6 mm, 7 mm, 8 mm, 9 mm, 10 mm, 11 mm, 12 mm, 13 mm, 14 mm, 15 mm, 16 mm or 17 mm tablet. The size refers to the diameter of the tablet, as is known in the art. As used herein, the size of the tablet refers to the size of the tablet prior to application of an enteric coating.

In some embodiments, the solid dosage form comprises a mini-tablet. The mini-tablet can be in the size range of 1 mm-4 mm range. E.g., the mini-tablet can be a 1 mm mini-tablet, 1.5 mm mini-tablet, 2 mm mini-tablet, 3 mm mini-tablet, or 4 mm mini-tablet. The size refers to the diameter of the mini-tablet, as is known in the art. As used herein, the size of the minitablet refers to the size of the mini-tablet prior to application of an enteric coating.

The mini-tablets can be in a capsule. The capsule can be a size 00, size 0, size 1, size 2, size 3, size 4, or size 5 capsule. The capsule that contains the mini-tablets can comprise a single layer coating, e.g., a non-enteric coating such as gelatin. The mini-tablets can be inside a capsule: the number of mini-tablets inside a capsule will depend on the size of the capsule and the size of the mini-tablets. As an example, a size 0 capsule can contain 31-35 (an average of 33) mini-tablets that are 3 mm mini-tablets.

The solid dosage form (e.g., tablet or mini-tablet or capsule) described herein can be enterically coated. In some embodiments, the enteric coating comprises HPMC (hydroxyl propyl methyl cellulose). In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P).

The solid dose form can comprise a coating. The solid dose form can comprise a single layer coating, e.g., enteric coating, e.g., a Eudragit-based coating, e.g., EUDRAGIT L30 D-55, triethylcitrate, and talc. The solid dose form can comprise two layers of coating. For example, an inner coating can comprise, e.g., EUDRAGIT L30 D-55, triethylcitrate, talc, citric acid anhydrous, and sodium hydroxide, and an outer coating can comprise, e.g., EUDRAGIT L30 D-55, triethylcitrate, and talc. EUDRAGIT is the brand name for a diverse range of polymethacrylate-based copolymers. It includes anionic, cationic, and neutral copolymers based on methacrylic acid and methacrylic/acrylic esters or their derivatives. Eudragits are amorphous polymers having glass transition temperatures between 9 to >150° C. Eudragits are non-biodegradable, nonabsorbable, and nontoxic. Anionic Eudragit L dissolves at pH >6 and is used for enteric coating, while Eudragit S, soluble at pH >7 is used for colon targeting. Eudragit RL and RS, having quaternary ammonium groups, are water insoluble, but swellable/permeable polymers which are suitable for the sustained release film coating applications. Cationic Eudragit E, insoluble at pH ≥5, can prevent drug release in saliva.

The solid dose form (e.g., a capsule) can comprise a single layer coating, e.g., a non-enteric coating such as gelatin.

A bacterial composition (e.g., pharmaceutical composition) comprising Prevotella histicola bacteria can be formulated as a suspension, e.g., for oral administration or for injection. Administration by injection includes intravenous (IV), intramuscular (IM), and subcutaneous (SC) administration. For a suspension, Prevotella histicola bacteria can be in a buffer, e.g., a pharmaceutically acceptable buffer, e.g., saline or PBS. The suspension can comprise one or more excipients, e.g., pharmaceutically acceptable excipients. The suspension can comprise, e.g., sucrose or glucose. The Prevotella bacteria in the suspension can be isolated Prevotella histicola bacteria. Optionally, the Prevotella histicola bacteria in the suspension can be lyophilized. Optionally, the Prevotella histicola bacteria in the solid dose form are live. Optionally, the Prevotella histicola bacteria in the solid dose form are non-viable. Optionally, the Prevotella histicola bacteria in the suspension can be gamma irradiated.

### Dosage

For oral administration to a human subject, the dose of Prevotella histicola bacteria can be, e.g., about 2×10⁶- about 2×10¹⁶ particles. The dose can be, e.g., about 1×10⁷- about 1×10¹⁵, about 1×10⁸- about 1×10¹⁴, about 1×10⁹- about 1×10¹³, about 1×10¹⁰- about 1×10¹⁴, or about 1×10⁸- about 1×10¹² particles. The dose can be, e.g., about 2×10⁶, about 2×10⁷, about 2×10⁸, about 2×10⁹, about 1×10¹⁰, about 2×10¹⁰, about 2×10¹¹, about 2×10¹², about 2×10¹³, about 2×10¹⁴, or about 1×10¹⁵ particles. The dose can be, e.g., about 2×10¹⁴ particles. The dose can be, e.g., about 2×10¹² particles. The dose can be, e.g., about 2×10¹⁰ particles. The dose can be, e.g., about 1×10¹⁰ particles. Particle count can be determined, e.g., by NTA.

For oral administration to a human subject, the dose of Prevotella histicola bacteria can be, e.g., based on total protein. The dose can be, e.g., about 5 mg to about 900 mg total protein. The dose can be, e.g., about 20 mg to about 800 mg, about 50 mg to about 700 mg, about 75 mg to about 600 mg, about 100 mg to about 500 mg, about 250 mg to about 750 mg, or about 200 mg to about 500 mg total protein. The dose can be, e.g., about 10 mg, about 25 mg, about 50 mg, about 75 mg, about 100 mg, about 150 mg, about 200 mg, about 250 mg, about 300 mg, about 400 mg, about 500 mg, about 600 mg, or about 750 mg total protein. The dose can be, e.g., about 10 mg total protein. Total protein can be determined, e.g., by Bradford assay or by the BCA assay.

For administration by injection (e.g., intravenous administration) to a human subject, the dose of Prevotella histicola bacteria can be, e.g., about 1×10⁶- about 1×10¹⁶ particles. The dose can be, e.g., about 1×10⁷- about 1×10¹⁵, about 1×10⁸- about 1×10¹⁴, about 1×10⁹- about 1×10¹³, about 1×10¹⁰- about 1×10¹⁴, or about 1×10⁸- about 1×10¹² particles. The dose can be, e.g., about 2×10⁶, about 2×10⁷, about 2×10⁸, about 2×10⁹, about 1×10¹⁰, about 2×10¹⁰, about 2×10¹¹, about 2×10¹², about 2×10¹³, about 2×10¹⁴, or about 1×10¹⁵ particles. The dose can be, e.g., about 1×10¹⁵ particles. The dose can be, e.g., about 2×10¹⁴ particles. The dose can be, e.g., about 2×10¹³ particles. Particle count can be determined, e.g., by NTA.

For administration by injection (e.g., intravenous administration), the dose of Prevotella histicola bacteria can be, e.g., about 5 mg to about 900 mg total protein. The dose can be, e.g., about 20 mg to about 800 mg, about 50 mg to about 700 mg, about 75 mg to about 600 mg, about 100 mg to about 500 mg, about 250 mg to about 750 mg, or about 200 mg to about 500 mg total protein. The dose can be, e.g., about 10 mg, about 25 mg, about 50 mg, about 75 mg, about 100 mg, about 150 mg, about 200 mg, about 250 mg, about 300 mg, about 400 mg, about 500 mg, about 600 mg, or about 750 mg total protein. The dose can be, e.g., about 700 mg total protein. The dose can be, e.g., about 350 mg total protein. The dose can be, e.g., about 175 mg total protein. Total protein can be determined, e.g., by Bradford assay or by the BCA assay.

In certain embodiments, the bacterial composition (e.g., pharmaceutical composition) comprises (e.g., comprises a total dose administered, e.g., once or twice daily, of) at least 1×10¹⁰ total cells (e.g., at least 1×10¹⁰ total cells, at least 2×10¹⁰ total cells, at least 3×10¹⁰ total cells, at least 4×10¹⁰ total cells, at least 5×10¹⁰ total cells, at least 6×10¹⁰ total cells, at least 7×10¹⁰ total cells, at least 8×10¹⁰ total cells, at least 9×10¹⁰ total cells, at least 1×10¹¹ total cells of the Prevotella histicola bacteria. In some embodiments, the pharmaceutical composition comprises no more than 9×10¹¹ total cells (e.g., no more than 1×10¹⁰ total cells, no more than 2×10¹⁰ total cells, no more than 3×10¹⁰ total cells, no more than 4×10¹⁰ total cells, no more than 5×10¹⁰ total cells, no more than 6×10¹⁰ total cells, no more than 7×10¹⁰ total cells, no more than 8×10¹⁰ total cells, no more than 9×10¹⁰ total cells, no more than 1×10¹⁰ total cells, no more than 2×10¹¹ total cells, no more than 3×10¹¹ total cells, no more than 4×10¹¹ total cells, no more than 5×10¹¹ total cells, no more than 6×10¹¹ total cells, no more than 7×10¹¹ total cells, no more than 8×10¹¹ total cells) of the Prevotella histicola bacteria. In some embodiments, the bacterial composition (e.g., pharmaceutical composition) comprises about 6×10⁹ total cells of the Prevotella histicola bacteria. In some embodiments, the bacterial composition (e.g., pharmaceutical composition) comprises about 1.6×10¹⁰ total cells of the Prevotella histicola bacteria. In some embodiments, the bacterial composition (e.g., pharmaceutical composition) comprises about 8×10¹⁰ total cells of the Prevotella histicola bacteria. In some embodiments, the bacterial composition (e.g., pharmaceutical composition) comprises about 1.6×10¹¹ total cells the Prevotella histicola bacteria. In some embodiments, the bacterial composition (e.g., pharmaceutical composition) comprises about 3.2×10¹¹ total cells the Prevotella histicola bacteria. In some embodiments, the bacterial composition (e.g., pharmaceutical composition) comprises about 8×10¹¹ total cells of the Prevotella histicola bacteria. In some embodiments, the bacterial composition (e.g., pharmaceutical composition) comprises about 1.6×10¹⁰ to about 8×10¹¹ total cells of the Prevotella histicola bacteria. In some embodiments, the bacterial composition (e.g., pharmaceutical composition) comprises about 1.6×10¹⁰ to about 1.6×10¹¹ total cells of the Prevotella histicola bacteria. In some embodiments, the bacterial composition (e.g., pharmaceutical composition) comprises about 8×10¹⁰ to about 8×10¹¹ total cells of the Prevotella histicola bacteria. In some embodiments, the bacterial composition (e.g., pharmaceutical composition) comprises about 1.6×10¹¹ to about 8×10¹¹ total cells of the Prevotella histicola bacteria.

In some embodiments, the Prevotella histicola bacteria may be quantified based on total cells, e.g., total cell count (TCC) (e.g., determined by Coulter counter).

In certain embodiments, provided herein are solid dosage forms comprising the Prevotella histicola bacteria. In some embodiments, the solid dosage form comprises an enteric coating. In some embodiments, the solid dosage form is a capsule, e.g., an enteric coated capsule. In some embodiments, each capsule comprises about 8×10¹⁰ total cells of the Prevotella histicola bacteria. In some embodiments, 1, 2, 3, 4, 5, 6, 7, 8, 9, or 10 capsules are administered, e.g., once or twice daily to a subject. In some embodiments, 1 capsule (e.g., comprising about 8×10¹⁰ total cells) is administered, e.g., once or twice daily to a subject. In some embodiments, 2 capsules (e.g., each comprising about 8×10¹⁰ total cells) are administered, e.g., once or twice daily to a subject. In some embodiments, 4 capsules (e.g., each comprising about 8×10¹⁰ total cells) are administered, e.g., once or twice daily to a subject. In some embodiments, 10 capsules (e.g., each comprising about 8×10¹⁰ total cells) are administered, e.g., once or twice daily to a subject. In some embodiments, the Prevotella histicola bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, the solid dosage form comprises a capsule. In some embodiments, the capsule is an enteric coated capsule. In some embodiments, the capsule comprises about 8×10¹⁰ total cells of the Prevotella histicola bacteria (e.g., total dose of a capsule or plurality of capsules). In some embodiments, the capsule comprises about 1.6×10¹¹ total cells of the Prevotella histicola bacteria (e.g., total dose of a capsule or plurality of capsules). In some embodiments, the capsule comprises about 3.2×10¹¹ total cells of the Prevotella histicola bacteria (e.g., total dose of a capsule or plurality of capsules). In some embodiments, the capsule comprises about 8×10¹¹ total cells of the Prevotella histicola bacteria (e.g., total dose of a capsule or plurality of capsules). In some embodiments, the Prevotella histicola bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, the solid dosage form comprises a tablet. In some embodiments, the tablet is an enteric coated tablet. In some embodiments, the enteric coated tablet is from 5 mm to 17 mm in diameter. In some embodiments, the tablet comprises about 8×10¹⁰ total cells of the Prevotella histicola bacteria (e.g., total dose of a tablet or plurality of tablets). In some embodiments, the tablet comprises about 1.6×10¹¹ total cells of the Prevotella histicola bacteria (e.g., total dose of a tablet or plurality of tablets). In some embodiments, the tablet comprises about 3.2×10¹¹ total cells of the Prevotella histicola bacteria (e.g., total dose of a tablet or plurality of tablets). In some embodiments, the tablet comprises about 8×10¹¹ total cells of the Prevotella histicola bacteria (e.g., total dose of a tablet or plurality of tablets). In some embodiments, the Prevotella histicola bacteria in the tablet are lyophilized (e.g., in a powder).

In some embodiments, the solid dosage form comprises a mini-tablet. In some embodiments, the mini-tablet is enteric coated. In some embodiments, the mini-tablet is from 1 mm to 4 mm in diameter. In some embodiments, the mini-tablet (e.g., enteric coated mini-tablet) is a 1 mm mini-tablet, 1.5 mm mini-tablet, 2 mm mini-tablet, 3 mm mini-tablet, or 4 mm mini-tablet. In some embodiments, the solid dosage form comprises mini-tablets that comprise about 8×10¹⁰ total cells of the Prevotella histicola bacteria (e.g., total dose of a plurality of mini-tablets). In some embodiments, the solid dosage form comprises mini-tablets that comprise about 1.6×10¹¹ total cells of the Prevotella histicola bacteria (e.g., total dose of a plurality of mini-tablets). In some embodiments, the solid dosage form comprises mini-tablets that comprise about 3.2×10¹¹ total cells of the Prevotella histicola bacteria (e.g., total dose of a plurality of mini-tablets). In some embodiments, the solid dosage form comprises mini-tablets that comprise about 8×10¹¹ total cells of the Prevotella histicola bacteria (e.g., total dose of a plurality of mini-tablets). In some embodiments, the Prevotella histicola bacteria in the mini-tablets are lyophilized (e.g., in a powder). In some embodiments, the mini-tablets (e.g., enteric coated mini-tablets) are contained in a capsule. In some embodiments, the capsule is a size 00, size 0, size 1, size 2, size 3, size 4, or size 5 capsule. In some embodiments, the capsule comprises (e.g., is coated with) a non-enteric coating (e.g., gelatin). In some embodiments, the capsule comprises a non-enteric coating. In some embodiments, the capsule comprises gelatin. In some embodiments, the mini-tablets (e.g., enteric coated mini-tablets) that comprise about 8×10¹¹ total cells of the Prevotella histicola bacteria are contained in a capsule(s), wherein optionally the capsule comprises gelatin.

### Gamma-Irradiation

Powders (e.g., of Prevotella histicola bacteria) can be gamma-irradiated at 17.5 kGy radiation unit at ambient temperature.

Frozen biomasses (e.g., of Prevotella histicola bacteria) can be gamma-irradiated at 25 kGy radiation unit in the presence of dry ice.

### Therapeutic Agents

In certain aspects, the methods provided herein include the administration to a subject of a bacterial composition described herein either alone or in combination with an additional therapeutic. In some embodiments, the additional therapeutic is an immunosuppressant, or a steroid.

In some embodiments the Prevotella histicola bacteria are administered to the subject before the therapeutic is administered (e.g., at least 1, 2, 3, 4, 5, 6, 7, 8, 9, 10, 11, 12, 13, 14, 15, 16, 17, 18, 19, 20, 21, 22, 23, or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days before). In some embodiments the Prevotella histicola bacteria are administered to the subject after the therapeutic is administered (e.g., at least 1, 2, 3, 4, 5, 6, 7, 8, 9, 10, 11, 12, 13, 14, 15, 16, 17, 18, 19, 20, 21, 22, 23, or 24 hours after or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after). In some embodiments, the Prevotella histicola bacteria and the therapeutic are administered to the subject simultaneously or nearly simultaneously (e.g., administrations occur within an hour of each other). In some embodiments, the subject is administered an antibiotic before the Prevotella bacteria are administered to the subject (e.g., at least 1, 2, 3, 4, 5, 6, 7, 8, 9, 10, 11, 12, 13, 14, 15, 16, 17, 18, 19, 20, 21, 22, 23, or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days before). In some embodiments, the subject is administered an antibiotic after the Prevotella bacteria are administered to the subject (e.g., at least 1, 2, 3, 4, 5, 6, 7, 8, 9, 10, 11, 12, 13, 14, 15, 16, 17, 18, 19, 20, 21, 22, 23, or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after). In some embodiments, the Prevotella bacteria and the antibiotic are administered to the subject simultaneously or nearly simultaneously (e.g., administrations occur within an hour of each other).

In some aspects, antibiotics can be selected based on their bactericidal or bacteriostatic properties. Bactericidal antibiotics include mechanisms of action that disrupt the cell wall (e.g., P-lactams), the cell membrane (e.g., daptomycin), or bacterial DNA (e.g., fluoroquinolones). Bacteriostatic agents inhibit bacterial replication and include sulfonamides, tetracyclines, and macrolides, and act by inhibiting protein synthesis. Furthermore, while some drugs can be bactericidal in certain organisms and bacteriostatic in others, knowing the target organism allows one skilled in the art to select an antibiotic with the appropriate properties. In certain treatment conditions, bacteriostatic antibiotics inhibit the activity of bactericidal antibiotics. Thus, in certain embodiments, bactericidal and bacteriostatic antibiotics are not combined.

Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

Aminoglycosides include, but are not limited to amikacin, gentamicin, kanamycin, neomycin, netilmicin, tobramycin, paromomycin, and spectinomycin. Aminoglycosides are effective, e.g., against Gram-negative bacteria, such as Escherichia coli, Klebsiella, Pseudomonas aeruginosa, and Francisella tularensis, and against certain aerobic bacteria but less effective against obligate/facultative anaerobes. Aminoglycosides are believed to bind to the bacterial 30S or 50S ribosomal subunit thereby inhibiting bacterial protein synthesis.

Ansamycins include, but are not limited to, geldanamycin, herbimycin, rifamycin, and streptovaricin. Geldanamycin and herbimycin are believed to inhibit or alter the function of Heat Shock Protein 90.

Carbacephems include, but are not limited to, Loracarbef. Carbacephems are believed to inhibit bacterial cell wall synthesis.

Carbapenems include, but are not limited to, Ertapenem, Doripenem, Imipenem/Cilastatin, and Meropenem. Carbapenems are bactericidal for both Gram-positive and Gram-negative bacteria as broad-spectrum antibiotics. Carbapenems are believed to inhibit bacterial cell wall synthesis.

Cephalosporins include, but are not limited to, Cefadroxil, Cefazolin, Cefalotin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, and Ceftobiprole. Selected Cephalosporins are effective, e.g., against Gram-negative bacteria and against Gram-positive bacteria, including Pseudomonas, certain Cephalosporins are effective against methicillin-resistant Staphylococcus aureus (MRSA). Cephalosporins are believed to inhibit bacterial cell wall synthesis by disrupting synthesis of the peptidoglycan layer of bacterial cell walls.

Glycopeptides include, but are not limited to, Teicoplanin, Vancomycin, and Telavancin. Glycopeptides are effective, e.g., against aerobic and anaerobic Gram-positive bacteria including MRSA and Clostridium difficile. Glycopeptides are believed to inhibit bacterial cell wall synthesis by disrupting synthesis of the peptidoglycan layer of bacterial cell walls.

Lincosamides include, but are not limited to, Clindamycin and Lincomycin. Lincosamides are effective, e.g., against anaerobic bacteria, as well as Staphylococcus and Streptococcus. Lincosamides are believed to bind to the bacterial 50S ribosomal subunit thereby inhibiting bacterial protein synthesis.

Lipopeptides include, but are not limited to, Daptomycin. Lipopeptides are effective, e.g., against Gram-positive bacteria. Lipopeptides are believed to bind to the bacterial membrane and cause rapid depolarization.

Macrolides include, but are not limited to, Azithromycin, Clarithromycin, Dirithromycin, Erythromycin, Roxithromycin, Troleandomycin, Telithromycin, and Spiramycin. Macrolides are effective, e.g., against Streptococcus and Mycoplasma. Macrolides are believed to bind to the bacterial or 50S ribosomal subunit, thereby inhibiting bacterial protein synthesis.

Monobactams include, but are not limited to, Aztreonam. Monobactams are effective, e.g., against Gram-negative bacteria. Monobactams are believed to inhibit bacterial cell wall synthesis by disrupting synthesis of the peptidoglycan layer of bacterial cell walls.

Nitrofurans include, but are not limited to, Furazolidone and Nitrofurantoin.

Oxazolidonones include, but are not limited to, Linezolid, Posizolid, Radezolid, and Torezolid. Oxazolidonones are believed to be protein synthesis inhibitors.

Penicillins include, but are not limited to, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin and Ticarcillin. Penicillins are effective, e.g., against Gram-positive bacteria, facultative anaerobes, e.g., Streptococcus, Borrelia, and Treponema. Penicillins are believed to inhibit bacterial cell wall synthesis by disrupting synthesis of the peptidoglycan layer of bacterial cell walls.

Penicillin combinations include, but are not limited to, Amoxicillin/clavulanate, Ampicillin/sulbactam, Piperacillin/tazobactam, and Ticarcillin/clavulanate.

Polypeptide antibiotics include, but are not limited to, Bacitracin, Colistin, and Polymyxin B and E. Polypeptide Antibiotics are effective, e.g., against Gram-negative bacteria. Certain polypeptide antibiotics are believed to inhibit isoprenyl pyrophosphate involved in synthesis of the peptidoglycan layer of bacterial cell walls, while others destabilize the bacterial outer membrane by displacing bacterial counter-ions.

Quinolones and Fluoroquinolone include, but are not limited to, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. Quinolones/Fluoroquinolone are effective, e.g., against Streptococcus and Neisseria. Quinolones/Fluoroquinolone are believed to inhibit the bacterial DNA gyrase or topoisomerase IV, thereby inhibiting DNA replication and transcription.

Sulfonamides include, but are not limited to, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole), and Sulfonamidochrysoidine. Sulfonamides are believed to inhibit folate synthesis by competitive inhibition of dihydropteroate synthetase, thereby inhibiting nucleic acid synthesis.

Tetracyclines include, but are not limited to, Demeclocycline, Doxycycline, Minocycline, Oxytetracycline, and Tetracycline. Tetracyclines are effective, e.g., against Gram-negative bacteria. Tetracyclines are believed to bind to the bacterial 30S ribosomal subunit thereby inhibiting bacterial protein synthesis.

Anti-mycobacterial compounds include, but are not limited to, Clofazimine, Dapsone, Capreomycin, Cycloserine, Ethambutol, Ethionamide, Isoniazid, Pyrazinamide, Rifampicin, Rifabutin, Rifapentine, and Streptomycin.

Suitable antibiotics also include arsphenamine, chloramphenicol, fosfomycin, fusidic acid, metronidazole, mupirocin, platensimycin, quinupristin/dalfopristin, tigecycline, tinidazole, trimethoprim amoxicillin/clavulanate, ampicillin/sulbactam, amphomycin ristocetin, azithromycin, bacitracin, buforin II, carbomycin, cecropin Pl, clarithromycin, erythromycins, furazolidone, fusidic acid, Na fusidate, gramicidin, imipenem, indolicidin, josamycin, magainan II, metronidazole, nitroimidazoles, mikamycin, mutacin B-Ny266, mutacin B-JHl 140, mutacin J-T8, nisin, nisin A, novobiocin, oleandomycin, ostreogrycin, piperacillin/tazobactam, pristinamycin, ramoplanin, ranalexin, reuterin, rifaximin, rosamicin, rosaramicin, spectinomycin, spiramycin, staphylomycin, streptogramin, streptogramin A, synergistin, taurolidine, teicoplanin, telithromycin, ticarcillin/clavulanic acid, triacetyloleandomycin, tylosin, tyrocidin, tyrothricin, vancomycin, vemamycin, and virginiamycin.

In some embodiments, the additional therapeutic is an immunosuppressive agent, a DMARD, a pain-control drug, a steroid, a non-steroidal anti-inflammatory drug (NSAID), or a cytokine antagonist, and/or combinations thereof. Representative agents include, but are not limited to, cyclosporin, retinoids, corticosteroids, propionic acid derivatives, acetic acid derivatives, enolic acid derivatives, fenamic acid derivatives, Cox-2 inhibitors, lumiracoxib, ibuprophen, cholin magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966; rofecoxib, acetominophen, Celecoxib, Diclofenac, tramadol, piroxicam, meloxicam, tenoxicam, droxicam, lornoxicam, isoxicam, mefanamic acid, meclofenamic acid, flufenamic acid, tolfenamic, valdecoxib, parecoxib, etodolac, indomethacin, aspirin, ibuprophen, firocoxib, methotrexate (MTX), antimalarial drugs (e.g., hydroxychloroquine and chloroquine), sulfasalazine, Leflunomide, azathioprine, gold salts, minocycline, cyclophosphamide, D-penicillamine, minocycline, auranofin, tacrolimus, myocrisin, chlorambucil, TNF alpha antagonists (e.g., TNF alpha antagonists or TNF alpha receptor antagonists), e.g., ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MabThera®), ABATACEPT (Orencia®), TOCILIZUMAB (RoActemra/Actemra®), integrin antagonists (e.g., TYSABRI® (natalizumab)), IL-1 antagonists (e.g., ACZ885 (Ilaris)), Anakinra (Kineret®)), CD4 antagonists, IL-23 antagonists, IL-20 antagonists, IL-6 antagonists, BLyS antagonists (e.g., Atacicept, Benlysta®/LymphoStat-B® (belimumab)), p38 Inhibitors, CD20 antagonists (e.g., Ocrelizumab, Ofatumumab (Arzerra®)), interferon gamma antagonists (e.g., Fontolizumab), prednisolone, Prednisone, dexamethasone, Cortisol, cortisone, hydrocortisone, methylprednisolone, betamethasone, triamcinolone, beclometasome, fludrocortisone, deoxycorticosterone, aldosterone, Doxycycline, vancomycin, pioglitazone, SBI-087, SCIO-469, Cura-100, Oncoxin+Viusid, TwIF, Methoxsalen, Vitamin D-ergocalciferol, Milnacipran, Paclitaxel, rosig tazone, Tacrolimus (Prograf®), RADOOl, rapamune, rapamycin, fostamatinib, Fentanyl, XOMA 052, Fostamatinib disodium, rosightazone, Curcumin (Longvida™), Rosuvastatin, Maraviroc, ramipril, Milnacipran, Cobiprostone, somatropin, tgAAC94 gene therapy vector, MK0359, GW856553, esomeprazole, everolimus, trastuzumab, JAK1 and JAK2 inhibitors, pan JAK inhibitors, e.g., tetracyclic pyridone 6 (P6), 325, PF-956980, denosumab, IL-6 antagonists, CD20 antagonistis, CTLA4 antagonists, IL-8 antagonists, IL-21 antagonists, IL-22 antagonist, integrin antagonists (e.g., Tysarbri® (natalizumab)), VGEF antagnosits, CXCL antagonists, MMP antagonists, defensin antagonists, IL-1 antagonists (including IL-1 beta antagonsits), and IL-23 antagonists (e.g., receptor decoys, antagonistic antibodies, etc.).

In some embodiments, the additional therapeutic is an oral PDE4 inhibitor (such as apremilast). In some embodiments, the additional therapeutic is apremilast, etanercept, infliximab, adalimumab, ustekinumab, or secukinumab.

In some embodiments, the agent is an immunosuppressive agent. Examples of immunosuppressive agents include, but are not limited to, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines (e.g., vaccines used for vaccination where the amount of an allergen is gradually increased), cytokine inhibitors, such as anti-IL-6 antibodies, TNF inhibitors such as infliximab, adalimumab, certolizumab pegol, golimumab, or etanercept, and combinations thereof.

### Administration

In some embodiments, the bacterial composition is administered orally. In some embodiments, the administration to the subject once daily. In some embodiments, the bacterial composition is administered in 2 or more doses (e.g., 3 or more, 4 or more or 5 or more doses). In some embodiments, the administration to the subject of the two or more doses are separated by at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days.

In some embodiments, the bacterial composition is administered once daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In some embodiments, the bacterial composition is administered once daily for at least 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

In some embodiments, the bacterial composition is formulated as a capsule or a tablet. In some embodiments, the bacterial formulation comprises an enteric coating or micro encapsulation. In some embodiments, the capsule is an enteric coated capsule.

In some embodiments, the subject is a mammal. In some embodiments, the subject is a human. In some embodiments, the subject is a non-human mammal (e.g., a dog, a cat, a cow, a horse, a pig, a donkey, a goat, a camel, a mouse, a rat, a guinea pig, a sheep, a llama, a monkey, a gorilla, or a chimpanzee).

In some embodiments of the methods provided herein, the bacterial composition is administered in conjunction with the administration of an additional therapeutic. In some embodiments, the bacterial composition comprises Prevotella bacteria co-formulated with the additional therapeutic. In some embodiments, the bacterial composition is co-administered with the additional therapeutic. In some embodiments, the additional therapeutic is administered to the subject before administration of the bacterial composition (e.g., about 1, 2, 3, 4, 5, 6, 7, 8, 9, 10, 15, 20, 25, 30, 35, 40, 45, 50, or 55 minutes before, about 1, 2, 3, 4, 5, 6, 7, 8, 9, 10, 11, 12, 13, 14, 15, 16, 17, 18, 19, 20, 21, 22, or 23 hours before, or about 1, 2, 3, 4, 5, 6, 7, 8, 9, 10, 11, 12, 13, or 14 days before). In some embodiments, the additional therapeutic is administered to the subject after administration of the bacterial composition (e.g., about 1, 2, 3, 4, 5, 6, 7, 8, 9, 10, 15, 20, 25, 30, 35, 40, 45, 50, or 55 minutes after, about 1, 2, 3, 4, 5, 6, 7, 8, 9, 10, 11, 12, 13, 14, 15, 16, 17, 18, 19, 20, 21, 22, or 23 hours after, or about 1, 2, 3, 4, 5, 6, 7, 8, 9, 10, 11, 12, 13, or 14 days after). In some embodiments the same mode of delivery are used to deliver both the bacterial composition and the additional therapeutic. In some embodiments different modes of delivery are used to administer the bacterial composition and the additional therapeutic. For example, in some embodiments the bacterial composition is administered orally while the additional therapeutic is administered via injection (e.g., an intravenous, and/or intramuscular injection).

In certain embodiments, the bacterial compositions, dosage forms, and kits described herein can be administered in conjunction with any other conventional treatment. These treatments may be applied as necessary and/or as indicated and may occur before, concurrent with or after administration of the bacterial compositions, dosage forms, and kits described herein.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, and other compounds such as drugs being administered concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate. The dose of the bacterial compositions described herein may be appropriately set or adjusted in accordance with the dosage form, the route of administration, the degree or stage of a target disease, and the like. The general effective dose of the additional therapeutic may range between 0.01 mg/kg body weight/day and 1000 mg/kg body weight/day, between 0.1 mg/kg body weight/day and 1000 mg/kg body weight/day, 0.5 mg/kg body weight/day and 500 mg/kg body weight/day, 1 mg/kg body weight/day and 100 mg/kg body weight/day, or between 5 mg/kg body weight/day and 50 mg/kg body weight/day. The effective dose may be 0.01, 0.05, 0.1, 0.5, 1, 2, 3, 5, 10, 20, 30, 40, 50, 60, 70, 80, 90, 100, 200, 500, or 1000 mg/kg body weight/day or more, but the dose is not limited thereto.

In some embodiments, the dose administered to a subject is sufficient to prevent inflammation or disease (e.g., autoimmune disease, inflammatory disease, metabolic disease), or treat disease, e.g., delay its onset, ameliorate one or more symptom of the disease, lessen the severity of the inflammation or the disease (or a symptom thereof), or slow or stop its progression. One skilled in the art will recognize that dosage will depend upon a variety of factors including the strength of the particular compound employed, as well as the age, species, condition, and body weight of the subject. The size of the dose will also be determined by the route, timing, and frequency of administration as well as the existence, nature, and extent of any adverse side-effects that might accompany the administration of a particular compound and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications (e.g., for treatment and/or prevention), the dosages of the active agents used in accordance with the invention vary depending on the active agent, the age, weight, and clinical condition of the recipient patient, and the experience and judgment of the clinician or practitioner administering the therapy, among other factors affecting the selected dosage.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a bacterial composition, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response and/or the time period for a subject to clear the bacteria from normal tissue. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the bacteria from normal tissue; for example, the time period can be more than the time period for a subject to clear the bacteria from normal tissue, such as more than about a day, more than about two days, more than about three days, more than about five days, or more than about a week.

In some embodiments, the delivery of an additional therapeutic in combination with the bacterial composition described herein reduces the adverse effects and/or improves the efficacy of the additional therapeutic.

The effective dose of an additional therapeutic described herein is the amount of the therapeutic agent that is effective to achieve the desired therapeutic response for a particular patient, composition, and mode of administration, with the least toxicity to the patient. The effective dosage level can be identified using the methods described herein and will depend upon a variety of pharmacokinetic factors including the activity of the particular compositions administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. In general, an effective dose of an additional therapy will be the amount of the therapeutic agent which is the lowest dose effective to produce a therapeutic effect. Such an effective dose will generally depend upon the factors described above.

The toxicity of an additional therapy is the level of adverse effects experienced by the subject during and following treatment. Adverse events associated with additional therapy toxicity include, but are not limited to, abdominal pain, acid indigestion, acid reflux, allergic reactions, alopecia, anaphylaxis, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypermagnesemia, hypernatremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heartbeat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

### Immune Disorders

In some embodiments, the methods and compositions described herein relate to the treatment or prevention of a disease or disorder associated a pathological immune response, such as an autoimmune disease, an allergic reaction and/or an inflammatory disease. In some embodiments, the disease or disorder is an inflammatory bowel disease (e.g., Crohn's disease or ulcerative colitis). In some embodiments, the disease or disorder is psoriasis (e.g., mild to moderate psoriasis). In some embodiments, the disease or disorder is atopic dermatitis (e.g., mild to moderate atopic dermatitis).

The methods described herein can be used to treat any subject in need thereof. As used herein, a “subject in need thereof” includes any subject that has a disease or disorder associated with a pathological immune response (psoriasis (e.g., mild to moderate psoriasis) or atopic dermatitis (e.g., mild to moderate atopic dermatitis)), as well as any subject with an increased likelihood of acquiring a such a disease or disorder.

The compositions described herein can be used, for example, as a bacterial composition for preventing or treating (reducing, partially or completely, the adverse effects of) an autoimmune disease, such as chronic inflammatory bowel disease, systemic lupus erythematosus, psoriasis, muckle-wells syndrome, rheumatoid arthritis, multiple sclerosis, or Hashimoto's disease; an allergic disease, such as a food allergy, pollenosis, or asthma; an infectious disease, such as an infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a bacterial composition for suppressing rejection in organ transplantation or other situations in which tissue rejection might occur; a supplement, food, or beverage for improving immune functions; or a reagent for suppressing the proliferation or function of immune cells.

In some embodiments, the methods provided herein are useful for the treatment of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below. In some embodiments, the methods provided herein are useful for the treatment of Th1-mediated inflammation. In some embodiments, the methods provided herein are useful for the treatment of Th2-mediated inflammation. In some embodiments, the methods provided herein are useful for the treatment of Th17-mediated inflammation.

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular immune disorders refers to a immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis.

Examples of nervous system immune disorders which may be treated with the methods and compositions described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system immune disorders which may be treated with the methods and compositions described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system immune disorders which may be treated with the methods and compositions described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The methods and compositions described herein may be used to treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, good pasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, Ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and compositions described herein may be used to treat T-cell-mediated hypersensitivity diseases having an inflammatory component. Such conditions include, but are not limited to, contact hypersensitivity, contact dermatitis (including that due to poison ivy), uticaria, skin allergies, respiratory allergies (hay fever, allergic rhinitis, house dust mite allergy), and gluten-sensitive enteropathy (Celiac disease).

Other immune disorders which may be treated with the methods and compositions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, and stomatitis, transplant rejection (involving organs such as kidney, liver, heart, lung, pancreas (e.g., islet cells), bone marrow, cornea, small bowel, skin allografts, skin homografts, and heart valve xenografts, serum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosis, psoriasis, chronic obstructive pulmonary disease, and inflammation accompanying infectious conditions (e.g., sepsis).

In some aspects, bacterial compositions for use of treating psoriasis and/or atopic dermatitis are disclosed. In some aspects, a bacterial composition comprising Prevotella histicola, wherein the Prevotella histicola is a strain comprising at least 85% sequence identity to the nucleotide sequence of the Prevotella histicola Strain B 50329 (NRRL accession number B 50329) for use in treating psoriasis is described herein. In other aspects, a bacterial composition comprising Prevotella histicola, wherein the Prevotella histicola is a strain comprising at least 85% sequence identity to the nucleotide sequence of the Prevotella histicola Strain B 50329 (NRRL accession number B 50329) for use in treating atopic dermatitis is described herein.

In some aspects, uses of a bacterial composition for the preparation of a medicament for treating psoriasis (e.g., mild to moderate psoriasis) and/or atopic dermatitis (e.g., mild to moderate atopic dermatitis) are disclosed. In some aspects, use of a bacterial composition for the preparation of a medicament for treating psoriasis wherein the bacterial composition comprises Prevotella histicola, wherein the Prevotella histicola is a strain comprising at least 85% sequence identity to the nucleotide sequence of the Prevotella histicola Strain B 50329 (NRRL accession number B 50329) is described herein. In other aspects, use of a bacterial composition for the preparation of a medicament for treating atopic dermatitis wherein the bacterial composition comprises Prevotella histicola, wherein the Prevotella histicola is a strain comprising at least 85% sequence identity to the nucleotide sequence of the Prevotella histicola Strain B 50329 (NRRL accession number B 50329) is described herein.

Numerous embodiments are further provided that can be applied to any aspect of the present invention described herein. For example, in some embodiments, the Prevotella histicola is a strain comprising at least 99.9% sequence identity to the nucleotide sequence of the Prevotella histicola Strain B 50329 (NRRL accession number B 50329). In some embodiments, the Prevotella histicola is the Prevotella histicola Strain B 50329 (NRRL accession number B 50329). In some embodiments, the bacterial composition is administered orally. In some embodiments, the bacterial composition is formulated as a capsule or a tablet. In some embodiments, the capsule is an enteric coated capsule. In some embodiments, the capsule is an enteric coated tablet. In some embodiments, the bacterial composition comprises about 1.6×10¹⁰ total cells of Prevotella histicola. In some embodiments, the bacterial composition comprises at most about 1.6×10¹⁰ total cells of Prevotella histicola. In some embodiments, the bacterial composition comprises about 1.6×10¹¹ total cells of Prevotella histicola. In some embodiments, the bacterial composition comprises at most about 1.6×10¹¹ total cells of Prevotella histicola. In some embodiments, the bacterial composition comprises about 3.2×10¹¹ total cells of Prevotella histicola. In some embodiments, the bacterial composition comprises at most about 3.2×10¹¹ total cells of Prevotella histicola. In some embodiments, the bacterial composition comprises about 8×10¹¹ total cells of Prevotella histicola. In some embodiments, the bacterial composition comprises at most about 8×10¹¹ total cells of Prevotella histicola. In some embodiments, the bacterial composition comprises from about 1.6×10¹⁰ to about 8×10¹¹ total cells of Prevotella histicola. In some embodiments, the bacterial composition comprises from about 8×10¹⁰ to about 8×10¹¹ total cells of Prevotella histicola. In some embodiments, the bacterial composition comprises about 2.76 mg, about 55 mg, about 550 mg, or about 2.76 g of Prevotella histicola. In some embodiments, the bacterial composition is administered at least once daily. In some embodiments, the bacterial composition is administered once daily. In some embodiments, the bacterial composition is administered once daily for 15 continuous days. In some embodiments, the bacterial composition is administered once daily for 28 continuous days. In some embodiments, the bacterial composition is administered once daily for 16 weeks. In some embodiments, the psoriasis is mild to moderate psoriasis. In some embodiments, the atopic dermatitis is mild to moderate atopic dermatitis.

### EXAMPLES

### Example 1: A Phase 2, Multicenter, Randomized, Double-Blind, Placebo-Controlled, Parallel-Cohort, Dose-Ranging Study Investigating the Effect of Prevotella Strain B 50329 in the Treatment of Mild to Moderate Plaque Psoriasis

### Rationale:

A therapeutic agent that offers the potential of systemic immune system modulation following oral administration, without systemic exposure is being developed. Prevotella Strain B 50329 is a pharmaceutical preparation of a strain of Prevotella histicola isolated from a human duodenal biopsy: it has not been genetically modified.

Studies of Prevotella Strain B 50329 in vitro in a range of human and mouse assays and studies in vivo in model symptoms support the use of Prevotella Strain B 50329 in the treatment of inflammatory diseases including psoriasis.

Oral administration of Prevotella Strain B 50329 to mice results in striking pharmacodynamic effects on animal models of delayed-type hypersensitivity, fluorescein isothiocyanate cutaneous hypersensitivity, collagen-induced arthritis and experimental acute encephalomyelitis. The high degree of consistency of both effect and dose suggests the potential for clinical benefit across multiple type 1, type 2, and type 3 inflammatory conditions. No potentially related adverse effects were seen in the animals used in these experiments with daily dosing for up to 3 weeks or with alternate day dosing for over 7 weeks. Immunophenotyping ex vivo in these models shows increased regulatory T cell numbers and regulatory dendritic cells in spleen and mesenteric lymph nodes, as well as decreases in pro-inflammatory cytokines such as IL-23p40, IL-17, TNF, IL-6, and IL-13. Treatment also led to enhancement of gut intestinal barrier integrity, which is often disrupted in patients with inflammatory diseases. These effects on immune parameters have been observed both within and outside the GI tract, which demonstrates that host-microbe interactions in the gut can affect the immune response in peripheral tissues.

Psoriasis is a chronic immune-mediated type ⅓ inflammatory skin disease in which hyperactive T cells trigger excessive keratinocyte proliferation. This results in the formation of raised erythematous plaques with scaling. Psoriatic lesions can appear anywhere on the body but are most often seen on the knees, elbows, scalp, and lumbar area. Critical events in the inflammatory process include activation of Langerhans cells and T cells, selective trafficking of activated T cells to the skin, and induction of an inflammatory cytokine and chemokine cascade in skin lesions. Clinical data have validated the role of anti-TNFa, anti-IL-17, and anti-IL-23 therapy in moderate to severe psoriasis. For patients with mild to moderate psoriasis, therapy usually involves topical agents (topical corticosteroids, vitamin D3 analogs), with topical corticosteroids providing the greatest range of efficacy and a wide range of formulations. More recently, physicians are prescribing apremilast, a first-in-class oral PDE4 inhibitor, ahead of biological therapy, which includes etanercept, infliximab, adalimumab, ustekinumab, and secukinumab.

In another study, a total of 56 participants have participated in Cohorts 1-4, with 36 of these participants receiving Prevotella Strain B 50329 once daily; 16 of these were treated for 14 days (healthy volunteers) and 20 participants with psoriasis were treated for 28 days. Clinical responses, similar to apremilast and tofacitinib at the same time point, have been observed on the (psoriatic) LSS and PASI at Day 28. Furthermore, at the Day 42 follow-up visit, when participants were “off treatment” for 14 days, there was continued improvement in the pharmacodynamic response as measured by both LSS and PASI for participants administered the higher dose, but not the lower dose, suggesting a dose relationship on the durability of effect. The safety profile of Prevotella Strain B 50329 was similar to placebo, with no SAEs or AEs of severe intensity.

The evidence available so far suggests Prevotella Strain B 50329 is very well tolerated and it continues to undergo clinical development in mild to moderate psoriasis. A well-tolerated oral therapy could offer significant benefit in the treatment of psoriasis and it is presently anticipated that Prevotella Strain B 50329 would be used in established but early disease, before the use of biologic therapies.

This Phase 2 study has been designed to investigate the clinical safety and efficacy of Prevotella Strain B 50329 and to identify an optimal dose.

### Objectives:

Primary Objective: The primary objective of this study is to evaluate the safety and efficacy of 3 different doses of Prevotella Strain B 50329 for the treatment of psoriasis following daily dosing for 16 weeks.

### Secondary Objectives:

The secondary objectives of this study are the following:
To evaluate the efficacy dose response of Prevotella Strain B 50329 at Week 16
To evaluate the maximal clinical benefit of Prevotella Strain B 50329 at Week 16
To evaluate the optimal dose of Prevotella Strain B 50329 based on efficacy and safety up to Week 16
To evaluate the safety and tolerability of Prevotella Strain B 50329 (all dose levels) throughout the study

### Exploratory Objectives:

The exploratory objectives of this study are the following:
To evaluate the time to onset of clinical response to Prevotella Strain B 50329
To evaluate the effect of Prevotella Strain B 50329 treatment on patient-reported outcomes including quality of life and pain
To evaluate the effect of Prevotella Strain B 50329 treatment on biomarkers in blood
To evaluate the effect of Prevotella Strain B 50329 treatment on biomarkers in skin plaques
To evaluate the effect of Prevotella Strain B 50329 treatment on fecal microbiome composition

### Estimands:

### Primary Estimands

The primary estimand will be the effect of Prevotella Strain B 50329 on the percent change in PASI score from baseline to Week 16 in the modified intent-to-treat (mITT) set of all treated participants, regardless of deviations from the protocol. Only data collected while on treatment will be used to account for the intercurrent event of treatment discontinuation (hypothetical strategy based on adherence to treatment only). The posterior mean difference between each active dose and the pooled placebo will be estimated.

For the primary analysis, 2 supportive estimands will also be considered:
To assess the impact of intercurrent events related to protocol deviations believed to impact efficacy, a supportive analysis will be performed where all data collected after any such identified protocol deviations will be excluded. Participants who had a protocol deviation believed to impact efficacy prior to the first dose of treatment (hypothetical strategy based on adherence to treatment and the aspects of the protocol which impact efficacy) will be completely excluded from this analysis.
To assess the impact of treatment discontinuation, a supportive analysis will be performed in which all data collected during the study, including any data collected after treatment discontinuation will be included (treatment policy strategy).

### Summary of Secondary Estimands:

For all secondary estimands, the population of interest will be the mITT set.

| | Consideration of | |
|---|---|---|
| Endpoint | intercurrent events | Summary measure |
| Mean percentage | To include all data | Posterior mean difference |
| change from baseline | collected | between each active |
| in PASI Score at | prior to treatment | treatment group and pooled |
| Weeks 4, 8, and 12 | discontinuation, | placebo at each visit |
| | regardless of | |
| | protocol deviations | |
| Mean absolute change | To include all data | Posterior mean difference |
| from baseline in PASI | collected | between each active |
| Score at Weeks 4, 8, | prior to treatment | treatment group and pooled |
| 12, and 16 | discontinuation, | placebo at each visit |
| | regardless of | |
| | protocol deviations | |
| Achievement of | To include all data | Posterior odds ratio for |
| PASI-50 at Weeks 4, | collected | response between each |
| 8, 12, and 16 | prior to treatment | active treatment group |
| | discontinuation, | and pooled placebo at |
| | regardless of | each visit |
| | protocol deviations | |
| Time to first | To include all data | Hazard ratio for each active |
| achievement of PASI- | collected | group versus placebo |
| 50 | prior to treatment | |
| | discontinuation, | |
| | regardless of | |
| | protocol deviations | |
| Achievement of | To include all data | Proportion of response for |
| PASI-75, PASI-90 and | collected | each endpoint in each |
| PASI-100 at Week 16 | prior to treatment | treatment group at each |
| | discontinuation, | visit |
| | regardless of | |
| | protocol deviations | |
| Achievement of PGA | To include all data | Posterior odds ratio for |
| of 0 or 1 with a ≥2- | collected | response between each |
| point improvement | prior to treatment | active treatment group |
| from baseline at | discontinuation, | and pooled placebo |
| Week 16 | regardless of | |
| | protocol deviations | |
| Achievement of PGA | To include all data | Posterior odds ratio for |
| of 0 at Week 16 | collected | response between each |
| | prior to treatment | active treatment |
| | discontinuation, | group and pooled placebo |
| | regardless of | |
| | protocol deviations | |
| Mean percentage | To include all data | Posterior mean difference |
| change from baseline | collected | between each active |
| in PGA × BSA at | prior to treatment | treatment group and pooled |
| Weeks 4, 8, 12, and 16 | discontinuation, | placebo at each visit |
| | regardless of | |
| | protocol deviations | |
| Mean absolute change | To include all data | Posterior mean difference |
| from baseline in | collected | between each active |
| PGA × BSA at Weeks | prior to treatment | treatment group and pooled |
| 4, 8, 12, and 16 | discontinuation, | placebo at each visit |
| | regardless of | |
| | protocol deviations | |
| Mean percentage | To include all data | Posterior mean difference |
| change from baseline | collected | between each active dose |
| in LSS at Weeks 4, 8, | prior to treatment | and pooled placebo at each |
| 12, and 16 | discontinuation, | visit |
| | regardless of | |
| | protocol deviations | |
| Mean absolute change | To include all data | Posterior mean difference |
| from baseline in LSS | collected | between each active dose |
| at Weeks 4, 8, 12, and | prior to treatment | and pooled placebo at each |
| 16 | discontinuation, | visit |
| | regardless of | |
| | protocol deviations | |
| Mean percentage | To include all data | Posterior mean difference |
| change from baseline | collected | between each active dose |
| in DLQI score at | prior to treatment | and pooled placebo at each |
| Weeks 4, 8, 12, and 16 | discontinuation, | visit |
| | regardless of | |
| | protocol deviations | |
| Mean absolute change | To include all data | Posterior mean difference |
| from baseline in DLQI | collected | between each active dose |
| score at Weeks 4, 8, | prior to treatment | and pooled placebo at each |
| 12, and 16 | discontinuation, | visit |
| | regardless of | |
| | protocol deviations | |
| Mean percentage | To include all data | Posterior mean difference |
| change from baseline | collected | between each active dose |
| in mNAPSI total score | prior to treatment | and pooled placebo at each |
| at Weeks 4, 8, 12, and | discontinuation, | visit |
| 16 | regardless of | |
| | protocol deviations | |
| Mean absolute change | To include all data | Posterior mean difference |
| from baseline in | collected | between each active dose |
| mNAPSI total score at | prior to treatment | and pooled placebo at each |
| Weeks 4, 8, 12, and 16 | discontinuation, | visit |
| | regardless of | |
| | protocol deviations | |

### Study Population:

### Inclusion Criteria

Each participant must meet all the following criteria to be enrolled in this study:
1. Give written informed consent (signed and dated) and any authorizations required by local law and be able to comply with all study requirements.
2. Males or females ≥18 and ≤70 years old at the time of informed consent.
3. A documented diagnosis of plaque psoriasis for ≥6 months.
4. Have mild to moderate plaque psoriasis with plaque covering BSA of ≥3% and ≤10% and meet both of the following additional criteria:
   a. PASI score of ≥6 and ≤15, and
   b. PGA score of 2 or 3.
All parameters in this criterion should be reconfirmed at baseline visit prior to randomization.
5. Meet the following contraception criteria:
   a. Male participants:
      i. A male participant must agree to use contraception as detailed in Appendix 13.1 of this protocol during their participation in this study and for a period of 90 days after the last dose and refrain from donating sperm during this period.
   b. Female participants:
      i. A female participant is eligible to participate if she is not pregnant (Appendix 13.1), not breastfeeding, and at least 1 of the following conditions applies:
         1. Not a WOCBP as defined in Appendix 13.1, OR
         2. A WOCBP who agrees to follow the contraceptive guidance in Appendix 13.1 during their participation in this study, 28 days prior to the first dose and for at least 1 complete menstrual cycle (≥28 days) after the last dose.
6. Agrees to not increase their usual sun exposure during the study.

### Exclusion Criteria

Participants meeting any of the following criteria will be excluded from the study:
1. Have received Prevotella Strain B 50329 within the 3 months prior to screening.
2. Have a diagnosis of non-plaque psoriasis.
3. Plaque psoriasis restricted to scalp, palms and soles only.
4. Evidence of skin conditions that would interfere with psoriasis evaluation or treatment response (e.g., atopic dermatitis, fungal, or bacterial superinfection).
5. Having received systemic immunosuppressive therapy (MTX, apremilast, azathioprine, cyclosporine, 6-thioguanine, mercaptopurine, mycophenolate mofetil, hydroxyurea, and tacrolimus) within 4 weeks of first administration of study drug.
6. Unresponsive to prior use of biologics (including, but not limited to, TNFa inhibitors, natalizumab, efalizumab, anakinra, or agents that modulate B cells or T cells).
7. If prior biologic therapy and responsive, participants must have been off therapy for at least 12 months prior to first administration of study drug.
8. Has received phototherapy or any systemic medications/treatments that could affect psoriasis or PGA evaluation (including, but not limited to oral or injectable corticosteroids, retinoids, 1,25-dihydroxy vitamin D3 and analogues, psoralens, sulfasalazine, hydroxyurea, or fumaric acid derivatives) within 4 weeks first administration of study drug.
9. Currently receiving lithium, antimalarials, leflunomide, or IM gold, or have received lithium, antimalarials, IM gold, or leflunomide within 4 weeks first administration of study drug.
10. Have used topical medications/treatments that could affect psoriasis or PGA evaluation (including (but not limited to) high- and mid-potency corticosteroids, anthralin, calcipotriene, topical vitamin D derivatives, retinoids, tazarotene, methoxsalen, trimethylpsoralens, picrolimus, and tacrolimus) within 2 weeks of the first administration of study drug. Topical unmedicated emollients and low-potency topical corticosteroids are not excluded.
11. Gastrointestinal tract disease (e.g., short-bowel syndrome, diarrhea-predominant irritable bowel syndrome) that could interfere with GI delivery and transit time.
12. Active inflammatory bowel disease.
13. Active infection requiring systemic antiviral or antimicrobial therapy that will not be completed prior to Day 1 (Visit 2).
14. Has received live or live-attenuated vaccination within 6 weeks prior to screening or intends to have such a vaccination during the study.
15. Clinically significant abnormalities in screening laboratory values that would render a participant unsuitable for inclusion (per investigator judgment).
16. For women, serum creatinine ≥125 μmol/L (1.414 mg/dL); for men, serum creatinine ≥135 μmol/L (1.527 mg/dL).
17. ALT and AST >2×ULN.
18. Known history of or positive test for HIV, or active infection with hepatitis C or chronic hepatitis B.
19. History of clinically significant acute cardiac or cerebrovascular event within 6 months before screening (includes stroke, transient ischemic attack, and coronary heart disease [angina pectoris, myocardial infarction, heart failure, revascularization procedures]).
20. In the opinion of the investigator, evidence of clinically important cardiac conduction abnormalities at screening as judged by ECG.
21. Current acute or chronic inflammatory disease other than psoriasis or psoriatic arthritis (e.g., inflammatory bowel disease, rheumatoid arthritis, systemic lupus erythematosus).
22. Hypersensitivity to P histicola or to any of the excipients.
23. Active untreated mental or psychiatric disorder. Participants who are on stable dosing of medication for a mental or psychiatric disorder for at least 6 months before screening and whose treating physicians consider them to be mentally stable may be enrolled.
24. Any major or minor GI surgery within 6 months of screening.
25. Any major surgery within 6 months of screening.
26. Malignancy within 5 years, except for basal or squamous cell carcinoma of the skin or carcinoma in situ of the cervix that has been successfully treated.
27. Treatment with another investigational drug, biological agent, or device within 1 month of screening, or 5 half-lives of investigational agent, whichever is longer.
28. Initiating any OTC or prescription medication including vitamins, herbal supplements and nutraceuticals (e.g., supplements including high doses of probiotics and prebiotics as usually found in capsules/tablets/powders), except acetaminophen/paracetamol and anti-histamines, within 14 days prior to baseline or anticipates change in dosage for the duration of the study period. Note that probiotic and prebiotic foods that contain low doses are allowed (e.g., yoghurt, kefir, kombucha).
29. Blood donation of >100 mL within 30 days of screening or of >499 mL within 12 weeks of screening.
30. Unwillingness to comply with study procedures, including follow-up, as specified by this protocol, or unwillingness to cooperate fully with the investigator.
31. Have any other conditions, which, in the opinion of the investigator or sponsor, would make the participant unsuitable for inclusion or could interfere with the participant participating in or completing the study.

### Study Design:

This is a multicenter, randomized, double-blind, placebo-controlled, parallel-cohort, dose-ranging study of participants with mild to moderate plaque psoriasis, comprising a screening period of up to 4 weeks, a baseline visit, a treatment period of 16 weeks (8 planned study site visits), and a follow-up period of 4 weeks (1 planned study site visit at the end of study).

After eligibility is confirmed during the screening period, participants will be randomly assigned in a 1:1:1 ratio to 1 of the following 3 parallel cohorts:
Cohort 1: 0.8×10¹¹ cells of Prevotella Strain B 50329 or matching placebo administered as 1 PIC, once daily.
Cohort 2: 3.2×10¹¹ cells of Prevotella Strain B 50329 or matching placebo administered as 4 PICs, once daily.
Cohort 3: 8.0×10¹¹ cells of Prevotella Strain B 50329 or matching placebo administered as 10 PICs, once daily.

In each cohort, approximately 75 participants will be randomly assigned in a 2:1 ratio to receive either Prevotella Strain B 50329 or matching placebo once daily for 16 weeks.

An interim analysis may be performed after at least 50% of participants have completed at least 12 weeks of treatment.

After the planned 16 weeks of treatment, all participants will enter a 4-week post-treatment follow-up period and undergo end of treatment evaluations.

### Estimated Study Duration:

The maximum planned duration for each participant will be 24 weeks (including 11 scheduled study visits), and the duration of the study is defined for each participant as the date signed written informed consent is provided through the last follow-up visit.

### Efficacy Assessments:

The efficacy assessments will include the PASI score, the LSS, the National Psoriasis Foundation Psoriasis Score version of a static PGA, the percent of BSA involvement, the mNAPSI, the DLQI, the PSI, the SF-36 Bodily Pain Scale, the VAS Pain assessment, the vitality subscale of the SF-36 (to assess fatigue), and a fatigue VAS.

### Pharmacokinetic or Pharmacodynamic Assessments:

Pharmacokinetic assessments will be limited to a predose blood sample at baseline and another sample at the Week 16 visit (end of treatment).

Pharmacodynamic and biomarker assessments are exploratory endpoints and analytical results for biomarkers will not be included in the CSR. They will be reported separately from the CSR.

Pharmacodynamic and biomarker assessments include digital photography of up to 6 lesion sites, standard histologic assessments of skin plaque biopsies, mRNA transcription analysis of skin plaque biopsies, blood cytokine and chemokine analyses, and microbiome composition of the fecal microbiome.

### Safety and Tolerability Assessments:

Safety and tolerability assessments include monitoring AEs (including SAEs), monitoring concomitant medications, BSFS categorization (recorded in a stool diary), physical examinations, vital sign measurements, and ECGs.

### Study Drug, Dosage, and Route of Administration:

The study drug will be capsules containing Prevotella Strain B 50329 or matching capsules containing placebo.

There will be 3 dosing cohorts, with dosages of 1 capsule, 4 capsules, or 10 capsules; capsules of Prevotella Strain B 50329 each contain 8.0×10¹⁰ cells of Prevotella Strain B 50329, while placebo capsules contain no bacteria.

Participants will self-administer their doses of study drug orally in the morning with water.

### Sample Size:

The sample size of 225 participants in total, has been chosen to explore the tolerability and safety of Prevotella Strain B 50329. Although the study will use a model-based probability inference approach in a Bayesian framework, the following power calculation was also performed (using a basic frequentist approach) in order to give confidence that enough participants are available to find a clinically meaningful difference between active dose and placebo if the below assumptions are met.

The primary efficacy endpoint is the percent change from baseline in the PASI score at Week 16. Percent change from baseline relative to placebo will be estimated within the model as (percent change in active)−(percent change in placebo), with a negative value indicating a greater improvement for active than placebo. A percent change from baseline relative to placebo of at least 20% will be considered clinically meaningful. The pooled standard deviation across all doses is assumed to be 25%.

Participants in the placebo arm of each cohort will be pooled for the statistical analysis in order to compare active and control arms resulting in 75 participants randomized to the pooled placebo group and 50 participants randomized to each active treatment group (Prevotella Strain B 50329 0.8×10¹¹ cells, Prevotella Strain B 50329 3.2×10¹¹ cells, and Prevotella Strain B 50329 8.0×10¹¹ cells). Assuming that no more than 15% of participants will discontinue treatment before the Week 16 visit, at least 42 active and 21 placebo participants in each of the 3 cohorts are expected to provide data through the Week 16 visit.

Each pairwise comparison between pooled placebo and active dose would be expected to have more than 95% power to detect a difference between the treatment groups at the 5% significance level under the assumption that pooling the placebo groups is a valid strategy. If the 3 placebo cohorts are considered to be too heterogeneous for pooling into a single reference group, the power to detect a difference in each within-cohort pairwise comparison between active and placebo doses would be greater than 80%.

As the statistical inference for this study will focus on estimation rather than testing a formal hypothesis, no multiplicity adjustments of the different comparisons between groups in order to control the study-wise type I error rate will be performed.

Similarly, as there is no intention to use any interim analyses to stop the study early for efficacy, no adjustments for multiplicity will be made to account for any analyses performed as part of the interim analyses.

### Statistical Methods:

Analysis methods for key endpoints are briefly described below. Further details on all analyses will be described in the SAP.

No formal hypothesis will be tested. A model-based probability inference approach in a Bayesian framework will be used to guide decision-making around dose selection. Posterior estimates and 95% credible intervals (CrI) for the difference between each active dose and placebo will be produced for relevant primary and secondary endpoints.

Unless otherwise specified, missing data will be considered as missing at random and will be accounted for using mixed models for repeated measures, with all time points collected for the relevant endpoint included in the model. This includes data which is excluded due to collection after treatment discontinuation or after a protocol deviation as applicable to the definition of the estimands used in the analysis.

### Analysis Sets:

The mITT set will consist of all participants who were randomized to treatment and who received at least one dose of study treatment.

The PPS will consist of all mITT participants who were not replaced (following study withdrawal before the end of Week 4) and who do not have a protocol deviation that may impact efficacy with a start date for the deviation before initiation of study treatment.

The safety set will consist of all participants who received any study drug.

The mITT set will be the primary population of interest for the efficacy section, with some supportive analyses performed using the PPS. The safety set will be used for all safety summaries.

### Statistical Analysis Methodology:

Statistical analysis will be performed using SAS software Version 9.3 or later. Continuous variables will be summarized using the mean, standard deviation, median, minimum value, and maximum value. Categorical variables will be summarized using frequency counts and percentages. Time-to-event variables will be summarized using Kaplan-Meier estimates of the proportion of participants with the event at each visit. Data will be listed in data listings.

### Analysis of Primary Efficacy Endpoint:

The primary analysis will be performed using a Bayesian MMRM. The model will include parameters for treatment*visit and baseline PASI score*visit interactions. Body mass index, gender, and other baseline covariates will also be considered and included as parameters if found to be significant (p<0.05). The model will not include an intercept. The priors for all parameters in the model will be non-informative and follow a normal distribution with mean 0 and SD 1000. The prior for the variance-covariance matrix will follow an inverted Wishart distribution with degrees of freedom equal to the number of visits and an identity scale matrix.

The adjusted posterior mean percentage change from baseline and the associated 95% HDP CrI for each treatment at Week 16 will be reported, together with the adjusted mean difference from placebo and the associated 95% HDP CrI for each active dose at each visit and the probability that each treatment difference is less than 0%, −20%, −30%, and −50%.

### Analysis of Secondary Efficacy Endpoints:

All secondary analyses will be performed on the mITT set, excluding data collected after treatment discontinuation, without consideration of any protocol deviations. Dose will be treated as a categorical variable and no dose response modelling will be done. Comparisons of interest will be between individual Prevotella Strain B 50329 doses and placebo. All posterior probabilities and CrI calculated will be considered as descriptive with no further adjustments for multiplicity performed.

### Analyses of Exploratory Efficacy Endpoints

Exploratory endpoints will be summarized using the mITT population, with data collected after discontinuation of treatment excluded but without consideration of any protocol deviations. Analyses of biomarkers will be addressed in a data analysis plan outside the study SAP.

### Pharmacokinetic Analyses:

The number and percentage of participants who have a quantifiable concentration of Prevotella Strain B 50329 in their blood sample will be summarized using the safety set by visit. Placebo participants will be pooled into a single treatment group. If at least 20% of participants within a treatment group are found to have a quantifiable level at one of the visits, then the concentration will be summarized as a continuous variable for the relevant treatment group at that visit.

### Safety Analyses:

All safety endpoints will be tabulated or plotted by treatment group using the safety set. All safety analyses will use the pooled placebo. Further details will be described in the SAP.

### List of Abbreviations and Definitions of Terms

| Abbreviation | Definition |
|---|---|
| AE | adverse event |
| ALT | alanine aminotransferase |
| AST | aspartate aminotransferase |
| BSA | body surface area |
| BMI | body mass index |
| BSFS | Bristol Stool Form Scale |
| CFR | Code of Federal Regulations |
| CrI | credible interval(s) |
| CRP | C-reactive protein |
| CSR | clinical study report |
| CTCAE | Common Terminology Criteria for Adverse Events |
| DIC | deviance information criterion |
| DLQI | Dermatology Life Quality Index |
| ECG | electrocardiogram |
| eCRF | electronic case report form |
| EDC | electronic data capture |
| Prevotella Strain | investigational study drug |
| B 50329 | |
| EOS | end of study |
| FDA | US Food and Drug Administration |
| FSH | follicle-stimulating hormone |
| FUP | functional uniform prior |
| GCP | Good Clinical Practice |
| GI | gastrointestinal |
| HbsAg | hepatitis B surface antigen |
| HCG | human chorionic gonadotropin |
| HCV | hepatitis C virus |
| HDP | high-density probability |
| HIV | human immunodeficiency virus |
| HPMC | hydroxypropyl methylcellulose |
| HRT | hormone replacement therapy |
| IB | investigator's brochure |
| ICF | informed consent form |
| ICH | International Council for Harmonisation |
| IEC | independent ethics committee |
| IL | interleukin |
| IM | intramuscular |
| IFNγ | interferon gamma |
| IRB | institutional review board |
| IRE | Ireland |
| IRT | interactive response technology |
| LSS | lesion severity score |
| MedDRA | Medical Dictionary for Regulatory Activities |
| mITT | modified intent-to-treat |
| MMRM | mixed effects model with repeated measures |
| MTX | methotrexate |
| mNAPSI | modified Nail Psoriasis Severity Index |
| OTC | over-the-counter |
| PASI | Psoriasis Area and Severity Index |
| PCR | polymerase chain reaction |
| PDE4 | phosphodiesterase type 4 |
| PGA | Physician's Global Assessment |
| PIC | powder in capsule |
| PPS | per-protocol set |
| PSI | Psoriasis Symptom Inventory |
| QTcF | QT interval corrected using Fridericia's formula |
| SAE | serious adverse event |
| SAP | statistical analysis plan |
| SoA | schedule of study site activities |
| SUSAR | suspected unexpected serious adverse reaction |
| TEAE | treatment-emergent adverse event |
| TNFα | tumor necrosis factor alpha |
| ULN | upper limit of normal |
| USA | United States of America |
| VAS | visual analog scale |
| WHO | World Health Organization |
| WOCBP | woman/women of child-bearing potential |

### Introduction

A therapeutic agent that offers the potential of systemic immune system modulation following oral administration, without systemic exposure, is being developed. Prevotella Strain B 50329 is a pharmaceutical preparation of a strain of Prevotella histicola isolated from a human duodenal biopsy: it has not been genetically modified. Strains of the Prevotella genus of microbes have been found in all human populations tested to date, at abundances ranging from less than 1% to nearly 50% of total fecal microbial load (Vandeputte 2017). Prevotella are gram-negative, obligate anaerobes that are natural human commensals in the oral cavity and GI tract. Prevotella Strain B 50329 is a gram-negative bacterium sensitive to the major classes of antibiotics, e.g., penicillins and cephalosporins. In non-clinical and clinical studies, its therapeutic effects have been dose-dependent.

Several studies (de Groot et al. 2017; Hindson et al. 2017; Yan et al. 2017; Felix et al 2018) suggest that host-microbe interactions in the gut, and particularly in the small intestine, can influence systemic inflammation. Preclinical data confirms that individual strains of microbes exhibit unique pharmacological profiles. This is thought to be based on multiple distinct microbial structural pattern motifs interacting with varying combinations of host pattern recognition receptors in small intestinal epithelium.

Studies of Prevotella Strain B 50329 in vitro in a range of human and mouse assays and studies in vivo in model symptoms support the use of Prevotella Strain B 50329 in the treatment of inflammatory diseases including psoriasis. Prevotella Strain B 50329 increases secretion of anti-inflammatory cytokines such as IL-10, IL1RA, and IL-27 from human immune cells, while inducing minimal production of pro-inflammatory cytokines such as IL-δ, TNFα, and IFNγ.

Oral administration of Prevotella Strain B 50329 to mice results in striking pharmacodynamic effects on animal models of delayed-type hypersensitivity, fluorescein isothiocyanate cutaneous hypersensitivity, collagen-induced arthritis (Marietta et al. 2016) and experimental acute encephalomyelitis (Mangalam et al. 2017). The high degree of consistency of both effect and dose suggests the potential for clinical benefit across multiple type 1, type 2, and type 3 inflammatory conditions. No potentially related adverse effects were seen in the animals used in these experiments with daily dosing for up to 3 weeks or with alternate day dosing for over 7 weeks. Immunophenotyping ex vivo in these models shows increased regulatory T cell numbers and regulatory dendritic cells in spleen and mesenteric lymph nodes, as well as decreases in pro-inflammatory cytokines such as IL-23p40, IL-17, TNF, IL-6, and IL-13. Treatment also led to enhancement of gut intestinal barrier integrity, which is often disrupted in patients with inflammatory diseases. These effects on immune parameters have been observed both within and outside the GI tract, which demonstrates that host-microbe interactions in the gut can affect the immune response in peripheral tissues.

Psoriasis is a chronic immune-mediated type ⅓ inflammatory skin disease in which hyperactive T cells trigger excessive keratinocyte proliferation. This results in the formation of raised erythematous plaques with scaling. Psoriatic lesions can appear anywhere on the body but are most often seen on the knees, elbows, scalp, and lumbar area. Critical events in the inflammatory process include activation of Langerhans cells and T cells, selective trafficking of activated T cells to the skin, and induction of an inflammatory cytokine and chemokine cascade in skin lesions. Clinical data have validated the role of anti-TNFα, anti-IL-17, and anti-IL-23 therapy in moderate to severe psoriasis. For patients with mild to moderate psoriasis, therapy usually involves topical agents (topical corticosteroids, vitamin D3 analogs), with topical corticosteroids providing the greatest range of efficacy and a wide range of formulations. More recently, physicians are prescribing apremilast, a first-in-class oral PDE4 inhibitor, ahead of biological therapy, which includes etanercept, infliximab, adalimumab, ustekinumab, and secukinumab.

In another study, a total of 56 participants have participated in Cohorts 1-4, with 36 of these participants receiving Prevotella Strain B 50329 once daily; 16 of these were treated for 14 days (healthy volunteers) and 20 participants with psoriasis were treated for 28 days. Clinical responses, similar to apremilast and tofacitinib at the same time point, have been observed on the (psoriatic) LSS and PASI at Day 28. Furthermore, at the Day 42 follow-up visit, when participants were “off treatment” for 14 days, there was continued improvement in the pharmacodynamic response as measured by both LSS and PASI for participants administered the higher dose, but not the lower dose, suggesting a dose relationship on the durability of effect. The safety profile of Prevotella Strain B 50329 was similar to placebo, with no SAEs or AEs of severe intensity.

The evidence available so far suggests Prevotella Strain B 50329 is very well tolerated and it continues to undergo clinical development in mild to moderate psoriasis. A well-tolerated oral therapy could offer significant benefit in the treatment of psoriasis and it is presently anticipated that Prevotella Strain B 50329 would be used in established but early disease, before the use of biologic therapies.

This Phase 2 study has been designed to investigate the clinical safety and efficacy of Prevotella Strain B 50329 and to identify an optimal dose.

### Study Objectives

All objectives are related to understanding the safety, efficacy, and dose effects of Prevotella Strain B 50329 treatment of mild to moderate plaque psoriasis in adult participants.

### Primary Objective

The primary objective of this study is to evaluate the safety and efficacy of 3 different doses of Prevotella Strain B 50329 for the treatment of psoriasis following daily dosing for 16 weeks.

### Secondary Objectives

The secondary objectives of this study are the following:
To evaluate the efficacy dose response of Prevotella Strain B 50329 at Week 16
To evaluate the maximal clinical benefit of Prevotella Strain B 50329 at Week 16
To evaluate the optimal dose of Prevotella Strain B 50329 based on efficacy and safety up to Week 16
To evaluate the safety and tolerability of Prevotella Strain B 50329 (all dose levels) throughout the study

### Exploratory Objectives

The exploratory objectives of this study are the following:
To evaluate the time to onset of clinical response to Prevotella Strain B 50329
To evaluate the effect of Prevotella Strain B 50329 treatment on patient-reported outcomes including quality of life and pain
To evaluate the effect of Prevotella Strain B 50329 treatment on biomarkers in blood
To evaluate the effect of Prevotella Strain B 50329 treatment on biomarkers in skin plaques
To evaluate the effect of Prevotella Strain B 50329 treatment on fecal microbiome composition

### Investigational Plan

### Study Design

This is a multicenter, randomized, double-blind, placebo-controlled, parallel-cohort, dose-ranging study of participants with mild to moderate plaque psoriasis, comprising a screening period of up to 4 weeks, a baseline visit, a treatment period of 16 weeks (8 planned study site visits), and a follow-up period of 4 weeks (1 planned study site visit at EOS). There are a total of 11 scheduled study visits.

After eligibility is confirmed during the screening period (as described herein), participants will be randomly assigned in a 1:1:1 ratio to 1 of the following 3 parallel cohorts:
Cohort 1: 0.8×10¹¹ cells of Prevotella Strain B 50329 or matching placebo administered as 1 PIC, once daily.
Cohort 2: 3.2×10¹¹ cells of Prevotella Strain B 50329 or matching placebo administered as 4 PICs, once daily.
Cohort 3: 8.0×10¹¹ cells of Prevotella Strain B 50329 or matching placebo administered as 10 PICs, once daily.

In each cohort, approximately 75 participants will be randomly assigned in a 2:1 ratio to receive either Prevotella Strain B 50329 or matching placebo once daily for 16 weeks.

An interim analysis may be performed after at least 50% of participants have completed at least 12 weeks of treatment.

After the planned 16 weeks of treatment, all participants will enter a 4-week post-treatment follow-up period and undergo end of treatment evaluations. The maximum planned duration for each participant will be 24 weeks, and the duration of the study is defined for each participant as the date signed written informed consent is provided through the last follow-up visit. Participants will be considered to have completed the study with the completion of all phases of the study, culminating with their EOS follow-up visit.

### Rationale for Study Design

The Prevotella Strain B 50329 Phase 1 program evaluated doses of 1.6×10¹⁰ cells to 8.0×10¹¹ cells given daily for 2 weeks in healthy volunteers and doses of 1.6×10¹ cells and 8.0×10¹¹ cells given daily for 4 weeks to participants with mild to moderate psoriasis. All doses were found to be well tolerated and doses of both 1.6×10¹¹ and 8.0×10¹¹ cells induced clinically relevant reductions in signs and symptoms of plaque psoriasis and psoriasis lesion severity.

The doses tested in the program are based on predictions from the preclinical data and the clinical and biomarker data obtained in the Phase 1 study. All doses tested up to 8.0×10¹¹ cells have been equally well tolerated. No clear difference in efficacy was observed between the 1.6×10¹¹ cells and the 8.0×10¹¹ cells in the previous study over the 28-day dosing period, but at the 14-day follow up (Day 42) the participants (e.g., subjects) receiving the higher dose had a continued improvement in their psoriasis compared to participants who had received the lower dose. This suggests a more sustained and potentially deeper response in the high dose group. It is therefore proposed to include the lowest and highest feasible doses (based on capsule load) in this study to establish the dose response, the maximum clinical benefit, and to assess participant tolerability and acceptability of the doses tested.

The clinical response to Prevotella Strain B 50329 treatment will be evaluated using multiple assessments, facilitating appropriate selection of efficacy measures for future studies.

The use of a placebo comparator is appropriate for this participant population of individuals with mild to moderate plaque psoriasis for the following reasons:
The limited proven efficacy of other treatments (topical corticosteroids, vitamin D3 analogs, and apremilast) in patients with mild to moderate plaque psoriasis that could potentially serve as an active comparator
The limited duration of the study (maximum of 16 weeks of treatment) for each participant
A randomization ratio of 2:1 for Prevotella Strain B 50329 treatment to placebo treatment in each cohort

### Participant Selection and Withdrawal Criteria

### Selection of Study Population

Approximately 225 participants will be enrolled (randomly assigned to treatment) in multiple countries, including (but not limited to) sites in the United States, the United Kingdom, and Poland. Participants will be assigned to study treatment only if they meet all inclusion criteria and no exclusion criteria during screening.

Deviations from the inclusion and exclusion criteria are not allowed: adherence to the eligibility criteria as specified in the protocol is essential.

Screen failures are defined as participants who consent to participate in the clinical study but are not subsequently randomly assigned to study drug (Prevotella Strain B 50329 or placebo). A minimal set of screen failure information is required to be entered in the eCRF to ensure transparent reporting of screen failure participants to meet the Consolidated Standards of Reporting Trials publishing requirements and to respond to queries from regulatory authorities. Minimal information includes demography, screen failure details, eligibility criteria, and any SAEs.

Individuals who fail to satisfy inclusion and exclusion criteria at screening may be rescreened 1 additional time with the agreement of the medical monitor before rescreening. Participants may also be rescreened if they initially pass the screening assessments but go beyond the screening period time limit. In exceptional circumstances, the screening window can be extended on a case-by-case basis after consultation with the sponsor: such an exceptional extension will not be considered a protocol deviation.

### Inclusion Criteria

Each participant must meet all the following criteria to be enrolled in this study:
1. Give written informed consent (signed and dated) and any authorizations required by local law and be able to comply with all study requirements.
2. Males or females ≥18 and ≤70 years old at the time of informed consent.
3. A documented diagnosis of plaque psoriasis for ≥6 months.
4. Have mild to moderate plaque psoriasis with plaque covering body surface area (BSA) of ≥3% and ≤10% and meet both of the following additional criteria:
   a. PASI score of ≥6 and ≤15, and
   b. PGA score of 2 or 3.
All parameters in this criterion should be reconfirmed at baseline visit prior to randomization.
5. Meet the following contraception criteria:
   a. Male participants:
      i. A male participant must agree to use contraception as detailed in Appendix 13.1 of this protocol during their participation in this study and for a period of 90 days after the last dose and refrain from donating sperm during this period.
   b. Female participants:
      i. A female participant is eligible to participate if she is not pregnant (Appendix 13.1), not breastfeeding, and at least 1 of the following conditions applies:
         1. Not a WOCBP as defined in Appendix 13.1, OR
         2. A WOCBP who agrees to follow the contraceptive guidance in Appendix 13.1 during their participation in this study, 28 days prior to the first dose and for at least 1 complete menstrual cycle (≥28 days) after the last dose.
6. Agrees to not increase their usual sun exposure during the study.

### Exclusion Criteria

Participants meeting any of the following criteria will be excluded from the study:
1. Have received Prevotella Strain B 50329 within the 3 months prior to screening.
2. Have a diagnosis of non-plaque psoriasis.
3. Plaque psoriasis restricted to scalp, palms, and soles only.
4. Evidence of skin conditions that would interfere with psoriasis evaluation or treatment response (e.g., atopic dermatitis, fungal or bacterial superinfection).
5. Have received systemic immunosuppressive therapy (MTX, apremilast, azathioprine, cyclosporine, 6-thioguanine, mercaptopurine, mycophenolate mofetil, hydroxyurea, and tacrolimus) within 4 weeks of first administration of study drug.
6. Unresponsive to prior use of biologics (including, but not limited to, TNFa inhibitors, natalizumab, efalizumab, anakinra or agents that modulate B cells or T cells).
7. If prior biologic therapy and responsive, participants must have been off therapy for at least 12 months prior to first administration of study drug.
8. Have received phototherapy or any systemic medications/treatments that could affect psoriasis or PGA evaluation (including, but not limited to oral or injectable corticosteroids, retinoids, 1,25-dihydroxy vitamin D3 and analogues, psoralens, sulfasalazine, hydroxyurea, or fumaric acid derivatives) within 4 weeks of first administration of study drug.
9. Currently receiving lithium, antimalarials, leflunomide, or IM gold, or have received lithium, antimalarials, IM gold, or leflunomide within 4 weeks of first administration of study drug.
10. Have used topical medications/treatments that could affect psoriasis or PGA evaluation (including [but not limited to] high- and mid-potency corticosteroids [Appendix 13.2], anthralin, calcipotriene, topical vitamin D derivatives, retinoids, tazarotene, methoxsalen, trimethylpsoralens, picrolimus, and tacrolimus) within 2 weeks of the first administration of study drug. Topical unmedicated emollients and low-potency topical corticosteroids are not excluded.
11. Gastrointestinal tract disease (e.g., short-bowel syndrome, diarrhea-predominant irritable bowel syndrome) that could interfere with GI delivery and transit time.
12. Active inflammatory bowel disease.
13. Active infection requiring systemic antiviral or antimicrobial therapy that will not be completed prior to Day 1 (Visit 2).
14. Have received live or live-attenuated vaccination within 6 weeks prior to screening or intend to have such a vaccination during the study.
15. Clinically significant abnormalities in screening laboratory values that would render a participant unsuitable for inclusion (per investigator judgment).
16. For women, serum creatinine ≥125 μmol/L (1.414 mg/dL); for men, serum creatinine ≥135 μmol/L (1.527 mg/dL).
17. ALT and AST >2×ULN.
18. Known history of or positive test for HIV, or active infection with hepatitis C or chronic hepatitis B.
19. History of clinically significant acute cardiac or cerebrovascular event within 6 months before screening (includes stroke, transient ischemic attack, and coronary heart disease [angina pectoris, myocardial infarction, heart failure, revascularization procedures]).
20. In the opinion of the investigator, evidence of clinically important cardiac conduction abnormalities at screening as judged by ECG.
21. Current acute or chronic inflammatory disease other than psoriasis or psoriatic arthritis (e.g., inflammatory bowel disease, rheumatoid arthritis, systemic lupus erythematosus).
22. Hypersensitivity to P. histicola or to any of the excipients.
23. Active untreated mental or psychiatric disorder. Participants who are on stable dosing of medication for a mental or psychiatric disorder for at least 6 months before screening and whose treating physicians consider them to be mentally stable may be enrolled.
24. Any major or minor GI surgery within 6 months of screening.
25. Any major surgery within 6 months of screening.
26. Malignancy within 5 years, except for basal or squamous cell carcinoma of the skin or carcinoma in situ of the cervix that has been successfully treated.
27. Treatment with another investigational drug, biological agent, or device within 1 month of screening, or 5 half-lives of investigational agent, whichever is longer.
28. Initiating any OTC or prescription medication including vitamins, herbal supplements and nutraceuticals (e.g., supplements including high doses of probiotics and prebiotics as usually found in capsules/tablets/powders), except acetaminophen/paracetamol and anti-histamines, within 14 days prior to baseline or anticipates change in dosage for the duration of the study period. Note that probiotic and prebiotic foods that contain low doses are allowed (e.g., yoghurt, kefir, kombucha).
29. Blood donation of >100 mL within 30 days of screening or >499 mL within 12 weeks of screening.
30. Unwillingness to comply with study procedures, including follow-up, as specified by this protocol or unwillingness to cooperate fully with the investigator.
31. Have any other conditions, which, in the opinion of the investigator or sponsor, would make the participant unsuitable for inclusion or could interfere with the participant participating in or completing the study.

### Study Treatments

### Method of Assigning Participants to Treatment Groups

Participants will be randomly assigned at the baseline visit (Visit 2) to 1 of 3 cohorts (in a 1:1:1 allocation ratio) that are distinguishable to participants and study staff by the number of capsules administered per once-daily dose. Within the cohort, participants will be randomly assigned in a 2:1 allocation ratio to receive either Prevotella Strain B 50329 or matching placebo treatment (as described herein). Interactive response technology (IRT) will be used to administer the randomization schedule.

### Treatments Administered

Participants in each cohort (as described herein) will self-administer study drug doses orally in the morning with water, refraining from consuming acidic drinks 1 hour either side of dosing and from eating 2 hours before dosing and 1 hour after dosing. The composition of capsules is described herein. Strategy to improve compliance is presented herein.

### Identity of Study Drug

The Prevotella Strain B 50329 drug product is available as enteric-coated HPMC hard capsules in Swedish-Orange color. The Prevotella Strain B 50329 PIC consists of freeze-dried powder of P histicola, mannitol, magnesium stearate, and colloidal silicon dioxide. Each Prevotella Strain B 50329 PIC contains 8.0×10¹⁰ cells of P. histicola. The matching placebo is identical in appearance but do not contain P. histicola or any other bacteria. The placebo excipients include microcrystalline cellulose and magnesium stearate.

### Management of Clinical Supplies

### Study Drug Packaging and Storage

Prevotella Strain B 50329 PICs and matching placebo will be prepared in blister wallets of 10 capsules. Blister wallets will be packaged in packs that contain approximately 1 week's supply of study drug for 1 randomized participant, identified by a numeric code. When appropriate for the interval between study visits, multiple packs will be assigned and dispensed for each participant throughout the treatment period.

Study drug (Prevotella Strain B 50329 and placebo) must be stored in a secure area (e.g., a locked refrigerator) and kept at a controlled temperature of 2° C. to 8° C. The investigator or designee must confirm appropriate temperature conditions have been maintained during transit and during storage at each site for all study drug received and any discrepancies are reported and resolved before use of the study drug.

### Concomitant Therapy

Anti-histamines and acetaminophen/paracetamol following labeled dosing instructions are permitted for use at any time during the study. Topical unmedicated emollients and low-potency topical steroids are also permitted if participants were already using them as part of their care prior to study entry (exclusion criterion #10). Participants will be advised to continue to use these therapies as they were prior to study entry.

Non-live vaccines are permitted in this study.

Other concomitant medication may be considered on a case-by-case basis by the investigator in consultation with the medical monitor if required.

### Prohibited Concomitant Therapy

Prior therapies restricted for participants eligible for this study as detailed in the exclusion criteria (as described herein) are prohibited concomitant therapy during the study.

Live or live-attenuated vaccines are contra-indicated in this study.

### Efficacy Assessments

### Psoriasis Area and Severity Index Score

The PASI score will be assessed as described by Langley and Ellis (2004). The PASI is a physician assessment that combines the assessment of the severity of and area affected by psoriasis into a single score in the range 0 (no disease) to 72 (maximal disease). The absolute PASI score in this study is used as part of inclusion criterion #4. The PASI percentage response rates are efficacy endpoints (i.e., PASI-50, PASI-75, PASI-90, and PASI-100). For example, the percentage of participants who achieve a 75% or greater reduction in PASI score from baseline is represented by the PASI-75 value. Details of the PASI assessment will be provided in the study manual.

### Lesion Severity Score

The LSS is used to score the severity of psoriasis plaques (Patel and Tsui 2011). The dimensions of scaling, erythema, and plaque elevation are each scored on a scale from 0 to 4, and the total LSS is the numerical sum of the 3-dimensional scores observed at a single study visit.

### Physician's Global Assessment

The National Psoriasis Foundation Psoriasis Score version of a static PGA is calculated by averaging the total body erythema, induration, and desquamation scores (Feldman and Krueger 2005). Erythema, induration, and desquamation will be scored on a 6-point scale, ranging from 0 (clear) to 5 (severe): the total PGA score is defined as the average of the erythema, induration, and desquamation scores. Details of the PGA assessment will be provided in the study manual.

### Percent of Body Surface Area Involvement

The percent of BSA involvement will be estimated for each participant, where 1% is approximately the area of the participant's handprint (Walsh et al. 2013). Details of the BSA assessment will be provided in the study manual.

Walsh and colleagues proposed the product of the PGA and the BSA involvement as a simple and effective alternative for measuring severity of psoriasis in clinical trials (Walsh et al. 2013).

### Modified Nail Psoriasis Severity Index

The mNAPSI is a numeric, reproducible, objective, and simple tool for physicians to evaluate the severity of nail bed psoriasis and nail matrix psoriasis by area of involvement in the nail unit (Cassell et al. 2007). Details of conducting the mNAPSI will be provided in the study manual.

### Dermatology Life Quality Index

The DLQI is a patient reported outcomes instrument for assessing the impact of dermatologic conditions on patients' quality of life (Finlay and Khan 1994). Details of administering the DLQI will be provided in the study manual.

### Psoriasis Symptom Inventory

The PSI is a patient reported outcomes instrument that is used to assess the severity of plaque psoriasis symptoms (Bushnell et al. 2013). All symptoms (itch, redness, scaling, burning, cracking, stinging, flaking, and pain) are rated on a 5-point severity scale. The PSI demonstrated good construct validity and was sensitive to within-subject change (p<0.0001). Details of administering the PSI will be provided in the study manual.

### Pain

Pain will be assessed by the SF-36 Bodily Pain Scale (SF-36 BPS) and the VAS Pain (Hawker et al. 2011). Details of administering the pain assessments will be provided in the study manual.

### Fatigue

Consistent with a recent study of fatigue in psoriasis (Skoie et al. 2017), fatigue will be assessed by the vitality subscale of the SF-36 (van der Heijden et al. 2003) and a fatigue VAS (Wolfe 2004). Details of administering the fatigue assessments will be provided in the study manual.

### Histologic Assessment

Standard histology will be performed on skin plaque biopsies (including epidermal thickness, basal mitotic counts and immune cell infiltrates, immunohistochemistry) from approximately 15 participants in each cohort. Details will be provided in the study manual. The histologic evaluations are exploratory and are outside the scope of the CSR.

mRNA Transcription Analysis

An mRNA transcription analysis will be performed on the skin plaque biopsies.

### Blood Cytokine and Chemokine Analysis

Blood samples will be stimulated ex vivo and analyzed for levels of cytokines and chemokines, including IL-1 beta, IL-2, IL-4, IL-6, IL-8, IL-10, IL-12p40, IL-17A, TNFa (TNFα), and IFNγ (IFNg).

### Statistical Considerations

Analysis methods for key endpoints are described below. Further details on all analyses will be described in the SAP.

No formal hypothesis will be tested. A model-based probability inference approach in a Bayesian framework will be used to guide decision-making around dose selection. Posterior estimates and 95% CrI for the difference between each active dose and placebo will be produced for relevant primary and secondary endpoints.

Unless otherwise specified, missing data will be considered as missing at random and will be accounted for using mixed models for repeated measures, with all time points collected for the relevant endpoint included in the model. This includes data which is excluded due to collection after treatment discontinuation or after a protocol deviation as applicable to the definition of the estimands used in the analysis.

### Estimands and Intercurrent Events

### Primary Efficacy Estimand

The primary estimand will be the effect of Prevotella Strain B 50329 on the percent change in PASI score from baseline to Week 16 in the mITT set of all treated participants, regardless of deviations from the protocol. Only data collected while on treatment will be used to account for the intercurrent event of treatment discontinuation (hypothetical strategy based on adherence to treatment only). The posterior mean difference between each active dose and the pooled placebo will be estimated.

Percent change from baseline in PASI score at each visit will be calculated as:

$$100 * ( PASI score at Visit - baseline PASI score ) / baseline PASI score .$$

A negative percentage change from baseline will indicate an improvement.

For the primary analysis, 2 supportive estimands will also be considered:
To assess the impact of intercurrent events related to protocol deviations believed to impact efficacy, a supportive analysis will be performed where all data collected after any such identified protocol deviations will be excluded. Participants who had a protocol deviation believed to impact efficacy prior to the first dose of treatment (hypothetical strategy based on adherence to treatment and the aspects of the protocol which impact efficacy) will be completely excluded from this analysis.
To assess the impact of treatment discontinuation, a supportive analysis will be performed in which all data collected during the study, including any data collected after treatment discontinuation will be included (treatment policy strategy).

The primary analysis will be performed using a Bayesian MMRM as fully described herein. Data from visits prior to Week 16 will be included in the model and missing data will not be explicitly imputed.

Supportive analyses will also be performed in the same manner, using the 2 alternative estimands as defined above. These will explore the possible impact of the intercurrent events of treatment discontinuation and events relating to protocol deviations that may have an impact on efficacy.

### Secondary Efficacy Estimands

Estimands for the analyses of all secondary endpoints are shown in the following table.

TABLE
Summary of Secondary Estimands

| | | Consideration of | |
|---|---|---|---|
| Population | | intercurrent | |
| of interest | Endpoint | events | Summary measure |
| mITT set | Mean percentage | To include all | Posterior mean |
| | change from | data collected | difference between |
| | baseline in PASI | prior to treatment | each active treatment |
| | Score at Weeks | discontinuation, | group and pooled |
| | 4, 8, and 12 | regardless of | placebo at each |
| | | protocol | visit |
| | | deviations | |
| mITT set | Mean absolute | To include all | Posterior mean |
| | change from | data collected | difference between |
| | baseline in PASI | prior to treatment | each active treatment |
| | Score at Weeks | discontinuation, | group and pooled |
| | 4, 8, 12, and 16 | regardless of | placebo at each |
| | | protocol | visit |
| | | deviations | |
| mITT set | Achievement of | To include all | Posterior odds ratio |
| | PASI-50 at | data collected | for response between |
| | Weeks 4, 8, | prior to treatment | each active treatment |
| | 12, and 16 | discontinuation, | group and pooled |
| | | regardless of | placebo at each |
| | | protocol | visit |
| | | deviations | |
| mITT set | Time to first | To include all | Hazard ratio for |
| | achievement of | data collected | each active group |
| | PASI-50 | prior to treatment | versus placebo |
| | | discontinuation, | |
| | | regardless of | |
| | | protocol | |
| | | deviations | |
| mITT set | Achievement of | To include all | Proportion of response |
| | PASI-75, | data collected | for each endpoint in |
| | PASI-90 and | prior to treatment | each treatment group |
| | PASI-100 at | discontinuation, | at each visit |
| | Week 16 | regardless of | |
| | | protocol | |
| | | deviations | |
| mITT set | Achievement of | To include all | Posterior odds ratio for |
| | PGA of 0 or 1 | data collected | response between each |
| | with a ≥2-point | prior to treatment | active treatment group |
| | improvement | discontinuation, | and pooled placebo |
| | from baseline | regardless of | |
| | at Week 16 | protocol | |
| | | deviations | |
| mITT set | Achievement of | To include all | Posterior odds ratio for |
| | PGA of 0 at | data collected | response between each |
| | Week 16 | prior to treatment | active treatment group |
| | | discontinuation, | and pooled placebo |
| | | regardless of | |
| | | protocol | |
| | | deviations | |
| mITT set | Mean percentage | To include all | Posterior mean |
| | change from | data collected | difference between |
| | baseline in | prior to treatment | each active |
| | PGA × BSA at | discontinuation, | treatment group and |
| | Weeks 4, 8, 12, | regardless of | pooled placebo at |
| | and 16 | protocol | each visit |
| | | deviations | |
| mITT set | Mean absolute | To include all | Posterior mean |
| | change from | data collected | difference between |
| | baseline in | prior to treatment | each active |
| | PGA × BSA at | discontinuation, | treatment group and |
| | Weeks 4, 8, 12, | regardless of | pooled placebo at |
| | and 16 | protocol | each visit |
| | | deviations | |
| mITT set | Mean percentage | To include all | Posterior mean |
| | change from | data collected | difference between |
| | baseline in | prior to treatment | each active dose and |
| | LSS at Weeks | discontinuation, | pooled placebo at |
| | 4, 8, 12, and | regardless of | each visit |
| | 16 | protocol | |
| | | deviations | |
| mITT set | Mean absolute | To include all | Posterior mean |
| | change from | data collected | difference between |
| | baseline in | prior to treatment | each active |
| | LSS at Weeks | discontinuation, | dose and pooled |
| | 4, 8, 12, and | regardless of | placebo at |
| | 16 | protocol | each visit |
| | | deviations | |
| mITT set | Mean percentage | To include all | Posterior mean |
| | change from | data collected | difference between |
| | baseline in DLQI | prior to treatment | each active dose and |
| | score at Weeks | discontinuation, | pooled placebo at |
| | 4, 8, 12, and 16 | regardless of | each visit |
| | | protocol | |
| | | deviations | |
| mITT set | Mean absolute | To include all | Posterior mean |
| | change from | data collected | difference between |
| | baseline in DLQI | prior to treatment | each active dose and |
| | score at Weeks | discontinuation, | pooled placebo at |
| | 4, 8, 12, and 16 | regardless of | each visit |
| | | protocol | |
| | | deviations | |
| mITT set | Mean percentage | To include all | Posterior mean |
| | change from | data collected | difference between |
| | baseline in | prior to treatment | each active dose and |
| | mNAPSI total | discontinuation, | pooled placebo at |
| | score at Weeks | regardless of | each visit |
| | 4, 8, 12, and 16 | protocol | |
| | | deviations | |
| mITT set | Mean absolute | To include all | Posterior mean |
| | change from | data collected | difference between |
| | baseline in | prior to treatment | each active dose and |
| | mNAPSI total | discontinuation, | pooled placebo at |
| | score at Weeks | regardless of | each visit |
| | 4, 8, 12, and 16 | protocol | |
| | | deviations | |

Summaries and analyses of the secondary endpoints are detailed in full herein.

### Exploratory Endpoints

The exploratory endpoints include the following:
Percentage of participants achieving PASI-50, PASI-75, PASI-90, and PASI-100 at Weeks 4, 8, and 12
Percentage of participants achieving PGA of 0 or 1 with a ≥2-point improvement at Weeks 4, 8, and 12
Percentage of participants achieving PGA of 0 at Weeks 4, 8, and 12
Mean change from baseline in PSI quality of life scores at Weeks 12 and 16
Mean percentage change from baseline in PSI quality of life scores at Weeks 12 and 16
Mean change from baseline in Pain and Fatigue scores at Weeks 4, 8, 12, and 16
Mean percentage change from baseline in Pain and Fatigue scores at Weeks 4, 8, 12, and 16
Mean change from baseline in fasting blood glucose and fasting lipid panel at Weeks 8 and 16

Biomarker endpoints (statistical analysis to appear separately from the CSR) include the following:
Histological assessment of skin plaque biopsies (including epidermal thickness, basal mitotic counts and immune cell infiltrates) at Week 16 versus baseline
mRNA transcription analysis on skin plaque biopsies at Week 16 versus baseline
Blood cytokine and chemokine levels at Week 16 versus baseline
Microbiome composition (in feces) at Week 16 and Week 20 versus baseline

Exploratory endpoints will be summarized using the mITT set, with data collected after discontinuation of treatment excluded but without consideration of any protocol deviations.

### Sample Size Determination

The sample size of 225 participants in total, has been chosen to explore the tolerability and safety of Prevotella Strain B 50329. Although the study will use a model-based probability inference approach in a Bayesian framework, the following power calculation was also performed (using a basic frequentist approach) in order to give confidence that enough participants are available to find a clinically meaningful difference between active dose and placebo if the below assumptions are met.

The primary efficacy endpoint is the percent change from baseline in the PASI score at Week 16. Percent change from baseline relative to placebo will be estimated within the model (as described herein) as (percent change in active)−(percent change in placebo), with a negative value indicating a greater improvement for active than placebo. A percent change from baseline relative to placebo of at least 20% will be considered clinically meaningful. The pooled standard deviation across all doses is assumed to be 25%.

Participants in the placebo arm of each cohort will be pooled for the statistical analysis in order to compare active and control arms resulting in 75 participants randomized to the pooled placebo group and 50 participants randomized to each active treatment group (Prevotella Strain B 50329 0.8×10¹¹ cells, Prevotella Strain B 50329 3.2×10¹¹ cells, and Prevotella Strain B 50329 8.0×10¹¹ cells). Assuming that no more than 15% of participants will discontinue treatment before the Week 16 visit, at least 42 active and 21 placebo participants in each of the 3 cohorts are expected to provide data through the Week 16 visit.

Each pairwise comparison between pooled placebo and active dose would be expected to have more than 95% power to detect a difference between the treatment groups at the 5% significance level under the assumption that pooling the placebo groups is a valid strategy. If the 3 placebo cohorts are considered to be too heterogeneous for pooling into a single reference group, the power to detect a difference in each within-cohort pairwise comparison between active and placebo doses would be greater than 80%.

As the statistical inference for this study will focus on estimation rather than testing a formal hypothesis, no multiplicity adjustments of the different comparisons between groups in order to control the study-wise type I error rate will be performed.

Similarly, as there is no intention to use any interim analyses to stop the study early for efficacy (as described herein), no adjustments for multiplicity will be made to account for any analyses performed as part of the interim analyses.

### Analysis Sets

The following analysis sets will be used in the statistical analyses.

Enrolled set: The enrolled set will consist of all participants who sign the ICF.

mITT set: The mITT set will consist of all participants who were randomized to treatment and who received at least one dose of study treatment. Participants who withdraw from the study before the end of Week 4 and are replaced will be included in this analysis set. All analyses using the mITT will group participants according to randomized treatment.

PPS: The PPS will consist of all mITT participants who were not replaced (following study withdrawal before the end of Week 4) and who do not have a protocol deviation that may impact efficacy with a start date for the deviation before initiation of study treatment. Note that in the case of participants who have a protocol deviation with a potential impact on efficacy after initiation of treatment, the participant will remain in the PPS but all data collected after the protocol deviation occurred will be excluded from any analyses performed using the PPS. All analyses using the PPS will group participants according to treatment received at the start of the study.

Safety set: The safety set will consist of all participants who received any study drug. All analyses using the safety set will group participants according to treatment received. If participants received multiple treatments during the study, they will be assigned to treatment group in the following manner:
If participant received both active and placebo treatments, they will be assigned to the active treatment group.
If participant received 2 or more different active dose levels, they will be assigned to the treatment they received for the longest period.

The mITT set will be the primary population of interest for the efficacy section, with some supportive analyses performed using the PPS. The safety set will be used for all safety summaries.

### Analysis of Primary Efficacy Endpoint

The assumption that the 3 cohorts of placebo participants can be pooled into a single placebo group to be used as a control for all active doses will be examined using mean (±SD) plots of percent change in PAST score against time.

The primary analysis will be performed using a Bayesian MMRM. The model will include parameters for treatment*visit and baseline PASI score*visit interactions. Body mass index, gender, and other baseline covariates will also be considered and included as parameters if found to be significant (p<0.05). The model will not include an intercept. Visit will consist of 6 levels (Weeks 1, 2, 4, 8, 12, and 16) and treatment will consist of 4 levels (pooled placebo, Prevotella Strain B 50329 0.8×10¹¹ cells, Prevotella Strain B 50329 3.2×10¹¹ cells, and Prevotella Strain B 50329 8.0×10¹¹ cells) if the placebo pooling strategy is considered appropriate or 6 levels (Placebo matching Prevotella Strain B 50329 0.8×10¹¹ cells, Placebo matching Prevotella Strain B 50329 3.2×10¹¹ cells, Placebo matching Prevotella Strain B 50329 8.0×10¹¹ cells, Prevotella Strain B 50329 0.8×10¹¹ cells, Prevotella Strain B 50329 3.2×10¹¹ cells, and Prevotella Strain B 50329 8.0×10¹¹ cells) if the placebo pooling strategy is not considered appropriate.

The priors for all parameters in the model will be non-informative and follow a normal distribution with mean 0 and SD 1000. The prior for the variance-covariance matrix will follow an inverted Wishart distribution with degrees of freedom equal to the number of visits and an identity scale matrix. The choice of Wishart distribution is based on it being the conjugate prior of the inverse-covariance matrix of a multivariate-normal random vector.

If the assumption of similarity between the 3 placebo cohorts is considered appropriate, the placebo cohorts will be pooled, and a single placebo control group will be used for the pairwise differences for each active dose to placebo. If the assumption of similarity is considered inappropriate, each placebo dose will be included in the model as a separate dose level and pairwise comparisons between each active dose and placebo will be performed using only the matching placebo dose data for the relevant active dose.

The adjusted posterior mean percentage change from baseline and the associated 95% HDP CrI for each treatment at Week 16 will be reported, together with the adjusted mean difference from placebo and the associated 95% HDP CrI for each active dose at each visit and the probability that each treatment difference is less than 0%, −20%, −30%, and −50%.

Model checking and diagnostic plots, including posterior density plots of the posterior samples for all parameters in the model and residual plots to evaluate the distributional assumptions underlying the model, will be produced. The assumption that data are missing at random will be evaluated by plotting the mean percentage change in PASI score against visit, by treatment group, for the subgroups of participants who completed 16 weeks of study drug compared with those who discontinued study drug before the Week 16 visit.

If model checking and diagnostic plots show a violation of the assumptions underlying the analysis, alternative statistical methods will be considered, appropriate to the type of violation observed.

This primary analysis will be repeated using the 2 supportive estimands defined herein.

A further sensitivity analysis will be performed on the model with the primary estimand, in which participants who withdrew from study drug due to treatment failure (demonstrated by the participant commencing an oral agent, biological, or intermediate or high-potency topical therapy for plaque psoriasis) will have their percentage change from PASI imputed at all visits after study drug was discontinued as the maximum on-treatment value reached (i.e., worst score carried forward).

If the assumption of similarity between the placebo cohorts is supported, a supplementary analysis will be performed on the percent change from baseline to Week 16 in PASI score using a dose-response model on the pooled cohorts. The log-linear, 3-parameter, and 4-parameter Eₘₐₓ models will be fitted and compared, with the best fitting model (lowest DIC) selected for use in the outputs.

The dose-response model will be fitted to the data using Bayesian techniques with noninformative priors for E₀ and Eₘₐₓ and an FUP for ED50 (3- and 4-parameter models only) and the slope parameter m (4-parameter model only). The rationale for this choice of inference is that the FUP shrinks the dose response towards a flat line throughout the dose range, therefore providing more conservative estimates of the dose-response relationship compared to maximum likelihood (Bornkamp 2014). The models will be fully described in the SAP.

Based on the selected model, the posterior mean with associated 95% HDP CrI, for the difference from placebo for each active dose will be produced for the pairwise differences between each active dose and placebo, together with the posterior mean and 95% HDP CrI of the treatment difference from placebo for each active dose and posterior probabilities that difference from placebo is less than 0, −20%, −30%, and −50%. A further sensitivity analysis will be performed on the dose response model, in which participants who withdrew from study drug due to treatment will have their Week 16 percentage change from PASI imputed as 100% after study drug was discontinued.

Percent change from baseline in PASI score will be summarized by visit.

### Analysis of Secondary Efficacy Endpoints

All secondary analyses will be performed either using the pooled placebo group if the assumption of similarity for the placebo cohorts is considered appropriate or using the 3 cohort-level placebo groups if it is not considered appropriate.

Unless otherwise specified, all secondary analyses will be performed on the mITT set, excluding data collected after treatment discontinuation, without consideration of any protocol deviations. Dose will be treated as a categorical variable and no dose response modelling will be done. Comparisons of interest will be between individual Prevotella Strain B 50329 doses and placebo. All posterior probabilities and CrI calculated will be considered as descriptive with no further adjustments for multiplicity performed.

Data will be analyzed as collected and no imputation of missing data will be performed.

Mean percentage change from baseline in PASI score at Weeks 4, 8, and 12 will be analyzed as part of the MMRM for the primary estimand. The same statistics produced for the Week 16 time point will also be produced at Weeks 4, 8, and 12.

The following secondary endpoints will be analyzed in the same manner as described for the primary analysis:
Mean absolute change from baseline in PASI score at Weeks 4, 8, 12, and 16
Mean percentage change from baseline in LSS at Weeks 4, 8, 12, and 16
Mean absolute change from baseline in LSS at Weeks 4, 8, 12, and 16
Mean percentage change from baseline in PGA×BSA at Weeks 4, 8, 12, and 16
Mean absolute change from baseline in PGA×BSA at Weeks 4, 8, 12, and 16
Mean percentage change from baseline in DLQI score at Weeks 4, 8, 12, and 16
Mean absolute change from baseline in DLQI score at Weeks 4, 8, 12, and 16
Mean percentage change from baseline in mNAPSI total score at Weeks 4, 8, 12, and 16
Mean absolute change from baseline in mNAPSI total score at Weeks 4, 8, 12, and 16

For the PASI-50, a Bayesian generalized linear mixed effects model with a logit link function will be fitted using data from all visits. Treatment*visit and baseline PASI score*visit interactions will be included in the model as fixed effects. Body mass index, gender, and other baseline covariates will also be considered and fitted as fixed effects if found to be significant (p<0.05). Odds ratios and 95% HDP CrI for each active dose compared to placebo at each visit will be presented.

A sensitivity analysis for the PASI-50 will also be performed, in the same manner as described above, in which participants who withdraw from study drug before Week 16 due to treatment failure will be included in the model with the PASI-50 endpoint imputed as ‘not achieved’ at all visits after study drug withdrawal.

Percentage of participants achieving PGA of 0 or 1 with a ≥2-point improvement and percentage of participants achieving a PGA of 0 will be analyzed in the same manner as described above for PASI-50.

For the time to first achievement of PASI-50, a Bayesian Cox proportional hazards model will be fitted with treatment and baseline PASI score as covariates. Hazard ratios and 95% HDP CrI for each active dose compared to placebo will be presented.

### Analyses of Exploratory Efficacy Endpoints

Exploratory endpoints will be summarized using the mITT population, with data collected after discontinuation of treatment excluded but without consideration of any protocol deviations. Details of all analyses to be performed on the exploratory endpoints will be detailed in the SAP.

Analyses of biomarkers will be addressed in a data analysis plan outside the study SAP.

### Pharmacokinetic Analyses

The number and percentage of participants who have a quantifiable concentration of Prevotella Strain B 50329 in their blood sample will be summarized using the safety set by visit. Placebo participants will be pooled into a single treatment group. If at least 20% of participants within a treatment group are found to have a quantifiable level at one of the visits, then the concentration will be summarized as a continuous variable for the relevant treatment group at that visit.

### Interim Analyses

An interim analysis may be undertaken during the conduct of the study after at least 50% of participants have completed at least 12 weeks of treatment or withdrawn from treatment. The purpose of this analyses will be to aid in the planning of future studies and for a better understanding of the benefit/risk profile of Prevotella Strain B 50329.

For the interim analysis, unblinded aggregate results will be produced by an unblinded team for strategic planning use. These will not be shared with any study site staff, participants, or clinical monitors who will be involved in the collection and review of individual study data.

The interim analysis will look at the primary endpoint of percentage change from baseline in PASI score, secondary, and safety endpoints. The posterior predictive probability (Spiegelhalter et al 2004) of the percent change from baseline in PASI score being at least 20% lower in each active dose compared to the pooled placebo will also be calculated, using the estimates of treatment difference found at Week 12 using the Bayesian MMRM described for the primary analysis. If the posterior predictive probabilities for all active doses are found to be <30%, then the study may be stopped for futility.

No decisions regarding study conduct, other than the potential to stop the study early for futility, will be made based on these assessments and the study will not be stopped if superior efficacy is found. Outputs featuring unblinded treatment assignments will be created by the unblinded analysis group (to be included in the data dissemination plan).

### REFERENCES

Blake M R, Raker J M, Whelan K. Validity and reliability of the Bristol Stool Form Scale in healthy adults and patients with diarrhoea-predominant irritable bowel syndrome. Aliment Pharmacol Ther. 2016; 44:693-703.
Bornkamp B. Practical considerations for using functional uniform prior distributions for dose-response estimation in clinical trials. Biom J. 2014; 56(6):947-62.
Bushnell D M, Martin M L, McCarrier K, et al. Validation of the Psoriasis Symptom Inventory (PSI), a patient-reported outcome measure to assess psoriasis symptom severity. J Dermatolog Treat. 2013; 24(5):356-60.
Cassell S E, Bieber J D, Rich P, et al. The modified Nail Psoriasis Severity Index: validation of an instrument to assess psoriatic nail involvement in patients with psoriatic arthritis. J Rheumatol. 2007 January; 34(1):123-9.
de Groot P F, Belzer C, Aydin O, et al. Distinct fecal and oral microbiota composition in human type 1 diabetes, an observational study. PLoS One. 2017; 12(12):e0188475.
Feldman S R, Krueger G G. Psoriasis assessment tools in clinical trials. Ann Rheum Dis. 2005; 64(Suppl 2):ii65-8; discussion ii69-73.
Felix K M, Tahsin S, Wu H J. Host-microbiota interplay in mediating immune disorders. Ann N Y Acad Sci. 2018; 1417(1):57-70.
Finlay A Y, Khan G K. Dermatology Life Quality Index (DLQI): a simple practical measure for routine clinical use. Clin Exp Dermatol. 1994; 19:210-6.
Hawker G A, Mian S, Kendzerska T, et al. Measures of adult pain: Visual Analog Scale for Pain (VAS Pain), Numeric Rating Scale for Pain (NRS Pain), McGill Pain Questionnaire (MPQ), Short-Form McGill Pain Questionnaire (SF-MPQ), Chronic Pain Grade Scale (CPGS), Short Form-36 Bodily Pain Scale (SF-36 BPS), and Measure of Intermittent and Constant Osteoarthritis Pain (ICOAP). Arthritis Care Res (Hoboken). 2011; 63 Suppl 11:S240-52.
Hindson J. Multiple sclerosis: A possible link between multiple sclerosis and gut microbiota. Nat Rev Neurol. 2017; 13(12):705.
Human Microbiome Project Consortium. A framework for human microbiome research. Nature. 2012; 486:215-21.
Langley R G, Ellis C N. Evaluating psoriasis with Psoriasis Area and Severity Index, Psoriasis Global Assessment, and Lattice System Physician's Global Assessment. J Am Acad Dermatol. 2004; 51(4):563-9.
Mangalam A, Shahi S K, Luckey D, et al. Human gut-derived commensal bacteria suppress CNS inflammatory and demyelinating disease. Cell Rep. 2017; 20(6):1269-77.
Marietta E V, Murray J A, Luckey D H, et al. Human gut-derived Prevotella histicola suppresses inflammatory arthritis in humanized mice. Arthritis Rheumatol. 2016; 68(12):2878-88.
O'Donnell L J D, Virjee J, Heaton K W. Detection of pseudodiarrhoea by simple clinical assessment of intestinal transit rate. BMJ. 1990; 300:439-40.
Patel R V, Tsui C L. Evaluating psoriasis: a review of the assessments most commonly used in clinical trials. Psoriasis Forum. 2011; 17(4):259-66. doi: 10.1177/247553031117a00403.
Skoie I M, Dalen I, Ternowitz T, et al. Fatigue in psoriasis: a controlled study. Br J Dermatol. 2017; 177(2):505-12.
Spiegelhalter D J, Abrams K R, Myles J P. Bayesian approaches to clinical trials and health-care evaluation. Chichester: John Wiley and Sons Ltd; 2004. 408 p.
van der Heijden P G, van Buuren S, Fekkes M, et al. Unidimensionality and reliability under Mokken scaling of the Dutch language version of the SF-36. Qual Life Res. 2003; 12:189-98.
Vandeputte D, Falony G, Vieira-Silva S, et al. Stool consistency is strongly associated with gut microbiota richness and composition, enterotypes and bacterial growth rates. Gut. 2016; 65:57-62.
Vandeputte D, Kathagen G, D'hoe K, et al. Quantitative microbiome profiling links gut community variation to microbial load. Nature. 2017; 551:507-11.
Walsh J A, McFadden M, Woodcock J, et al. Product of the Physician Global Assessment and body surface area: a simple static measure of psoriasis severity in a longitudinal cohort. J Am Acad Dermatol. 2013; 69(6):931-7.
Wolfe F. Fatigue assessments in rheumatoid arthritis: comparative performance of visual analog scales and longer fatigue questionnaires in 7760 patients. J Rheumatol. 2004; 31(10):1896-902.
Yan D, Issa N, Afifi L, et al. The role of the skin and gut microbiome in psoriatic disease. Curr Dermatol Rep. 2017; 6:94-103.

### Example 2: Reductions of Cytokines

Treatment with Prevotella histicola Strain B led to reduced production of the following cytokines in blood cells: IL-1b, IL-2, IFNg, IL-4, IL-5, IL-6, IL-8, IL-10, TNFa and IL-17.

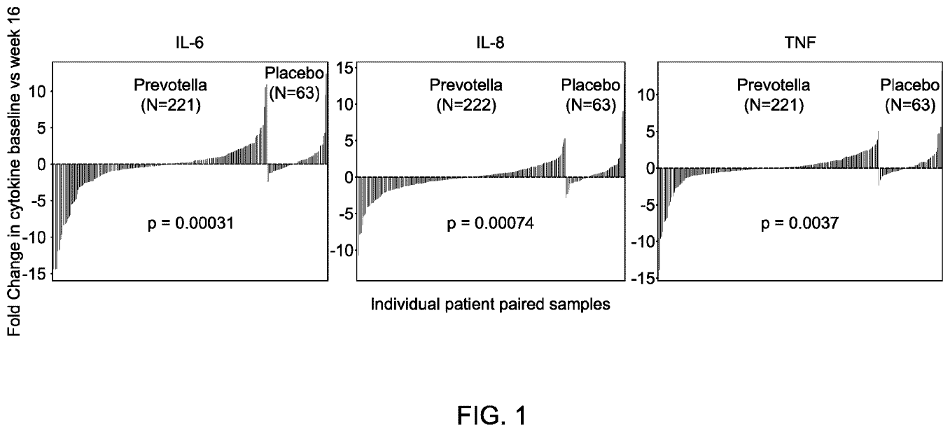

Blood samples were taken from 96 patients in the Phase 2 trial described in Example 1 at baseline and after 16 weeks of dosing with Prevotella histicola Strain B or placebo. FIG. 1 shows the changes in pro-inflammatory cytokines interleukin 6 (1L-6), interleukin 8 (IL-8) and tumor necrosis factor (TNF or TNFα). Each vertical bar represents the fold change up or down from 0 in ex vivo stimulated cytokine production between the baseline and week 16 samples from a patient. Three different stimuli were used on each sample and the results from all three stimuli are presented together in the figure, giving the aggregate N (sample) numbers shown in the figure.

Treatment with Prevotella histicola Strain B led to a statistically significant reduction in the release of cytokines compared to placebo: IL-6 (p=0.0003), IL-8 (p=0.0007), and TNFa (p=0.0037). The effect of Prevotella histicola Strain B is clearly seen by the deep tail of reduced cytokine production on the left of the distribution for each cytokine, which is absent in the placebo groups. There was no worsening compared to placebo on the right of the distributions, resulting in the overall significant difference between Prevotella histicola Strain B and placebo.

Treatment with Prevotella histicola Strain B also led to a statistically significant reduction in the release of cytokines compared to placebo for IL-1b, IL-2, IFNg, IL-4, IL-5, IL-10, and IL-17.

Ex vivo Stimulation Biomarker Analysis Protocol: Blood samples were taken from 96 patients at baseline and after 16 weeks of daily oral administration of Prevotella histicola Strain B (N=74) or placebo (N=22) in the Phase 2 clinical trial in mild and moderate psoriasis. Whole blood was incubated with three stimuli separately: lipopolysaccharide (LPS), antibodies to CD3 and CD28, and staphylococcal enterotoxin B (SEB), which cover a broad range of immune cell function. Analyses were performed on blood samples from 96 of the patients in the trial. Fifty-five of these patients achieved at least a PASI-50 at week 16 or had PASI scores greater than 150% worse than baseline at week 16, selected from both active and placebo treatment groups. The other 41 patients were drawn at random from active and placebo groups pro rata to assemble a representative sample of the trial population, enriched by both tails of PASI outcomes. The differences in ex vivo stimulated cytokine production at baseline and week 16 were calculated. Cytokine production was measured by Luminex.

### Example 3: Reductions in IL-23, IL-12b, IL-17 in Skin Lesions

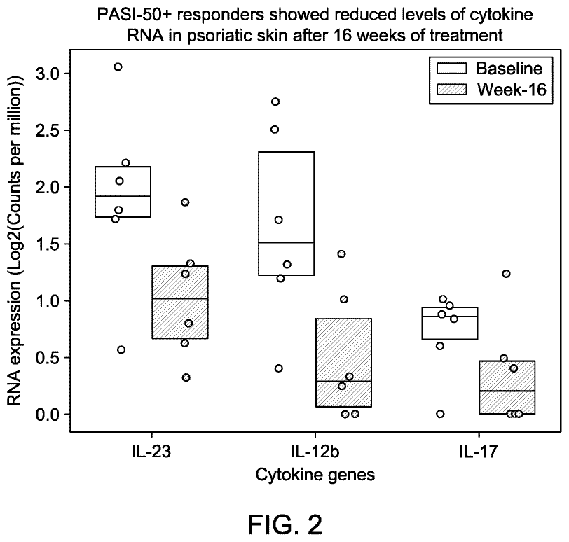

Skin biopsies of active lesions were taken from a subset of six patients in the Phase 2 trial described in Example 1 who received Prevotella histicola Strain B and achieved at least a 50% improvement in their Psoriasis Area and Severity Index (PASI) score (PASI-50) from baseline at week 16. RNAseq analysis showed reductions in transcript levels for psoriasis-relevant cytokines interleukin 23 (IL-23), interleukin 12b (IL-12b), and interleukin 17 (IL-17) in these lesions between baseline and week 16. The box plot in FIG. 2 shows the median and interquartile ranges, as well as individual values of the cytokine expression levels in the skin, at baseline and week 16. These data were consistent with the systemic effects in blood samples described in Example 2, suggesting that Prevotella histicola Strain B reduces inflammation in the skin by modulating multiple proinflammatory cytokines systemically.

### Example 4: Durable and Deepening Clinical Responses Observed in Post-Treatment Period of Phase 2 Clinical Trial of Oral Prevotella histicola Strain B in Psoriasis

Part B of the study is designed to assess the durability of treatment response and incidence of rebound of psoriasis following cessation of dosing. All participants will attend for skin assessments at Weeks 24 and 28, unless they have previously experienced treatment failure and/or had rebound of disease. A final visit at Week 40 will be performed for those participants who experience treatment response at Week 16 of treatment, but have not yet met the definition of disease relapse. On completion of Part B, the final analysis will be performed.

After the planned 16 weeks of treatment, all participants will have an initial follow-up visit at Week 20 (i.e., 4 weeks post last dose of study treatment), completing Part A of the study; and enter Part B with continued follow-up for a maximum of 20 additional weeks (up to Week 40). The duration of follow up will depend on the initial response to study therapy and any relapse or rebound experienced after cessation of study treatment.

Part B of the study, which includes additional visits at Weeks 24, 28, and 40, is designed to help understand the durability of treatment effect, including time to relapse and incidence of any rebound of psoriasis after treatment has completed. These are important aspects to understand regarding a psoriasis therapy (EMA guideline CHMP/EWP/2454/02, 2004).

Post-Treatment Follow-Up Assessments in Part B include: BSA involvement (%), PGA, PASI, LSS, mNAPSI, PSI, DLQI, and concomitant medication review; each to be assessed at week 24, week 28, and week 40 of the trial.

### Results:

18/30 patients maintained a PASI-50 or greater response at 24 weeks post-treatment
9/20 patients experienced a deepening of response from PASI-50 to at least PASI-75 during post-treatment period
No flare or rebound observed following discontinuation of treatment

The Phase 2 trial was comprised of a Part A, when patients received either Prevotella histicola Strain B or placebo for 16 weeks, and a Part B, when patients were followed for up to 24 weeks after they had stopped receiving Prevotella histicola Strain B or placebo. There were 83 patients who had received Prevotella histicola Strain B in Part A who entered Part B. Thirty of these 83 patients had achieved a PASI-50 (50% reduction in Psoriasis Area and Severity Index score from baseline) or greater reduction at week 16 of Part A. Eighteen of the 30 patients remained at PASI-50 or greater at the end of Part B. Ten of the 30 patients had achieved a PASI-75 or greater at the end of Part A and 5 remained at PASI-75 or greater at the end of Part B. These durable results were achieved without any new psoriasis medication being used during this time. Nineteen of the 83 patients had achieved clear skin (PGA 0) or nearly clear skin (PGA 1) at the end of Part A and of these, 9 remained at PGA 0/1 at the end of Part B.

Of the 30 patients who had reached a PASI-50 at the end of Part A and entered Part B, 10 had already achieved a PASI-75 response at week 16 in Part A. Of the remaining 20 patients, 9 achieved a PASI-75 or greater response during the post-treatment period. Three patients achieved PASI-100 for the first-time during the post-treatment period. These data, combined with the durability data, suggest that longer dosing could lead to further deepening of the responses in some patients.

The tolerability and safety data for Prevotella histicola Strain B in the trial was comparable to placebo, with the additional finding of no flare or rebound following discontinuation of therapy (which are often seen with other therapies for psoriasis).

### Additional Details of the Phase 2 Trial:

The trial was a multicenter, randomized, double-blind, placebo-controlled, parallel-cohort, dose-ranging trial in adult patients with mild and moderate psoriasis. The study included a Part A (treatment phase) and Part B (extended follow-up phase, off-treatment).

In Part A of the trial, 249 patients were randomized in a 1:1:1 ratio to one of the three parallel cohorts, 1 capsule, 4 capsule or 10 capsule. They were then randomized in a 2:1 ratio to active or placebo prior to starting dosing. Trial medication was taken once daily for 16 weeks, and patients were followed up for 4 weeks after treatment completion to week 20. In the trial, the PASI scores were assessed by both mean changes from baseline and responder rates. The primary endpoint was mean percentage reduction in PASI score at 16 weeks. Secondary endpoints included the proportion of trial participants who achieve a PASI-50 response or greater and other clinical measures of disease. The 16-week primary endpoint gave probabilities that Prevotella histicola Strain B was superior to placebo ranging from 80% to 90% across the prespecified analyses and cohorts. 25% to 32% of patients across the three cohorts who were treated with Prevotella histicola Strain B achieved a PASI-50 at week 16 compared to 12% on placebo.

All patients had the option to enter Part B of the trial. The objective of Part B was to assess durability of treatment response and incidence of rebound (e.g., increase in PASI score to 125% of baseline value or above, or onset of new pustular erythrodermic psoriasis within 3 months of cessation of dosing) following cessation of dosing. Patients in Part B were assessed during follow-up visits at weeks 24 and 28. Only patients who had achieved a PASI-50 or greater at week 16 were also evaluated at week 40. Patients were not permitted to start other psoriasis treatments or trials during Part B.

See also Example 1.

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of reducing IL-23, IL-12b, and/or IL-17 RNA levels in a subject in need thereof, comprising orally administering to the subject a bacterial composition comprising a Prevotella histicola strain comprising at least 99% genomic, 16S and/or CRISPR sequence identity to the nucleotide sequence of the Prevotella Strain B 50329 (NRRL accession number B 50329).

2. The method of claim 1, wherein the method reduces IL-23 RNA levels.

3. The method of claim 1, wherein the method reduces IL-12b RNA levels.

4. The method of claim 1, wherein the method reduces IL-17 RNA levels.

5. The method of any one of claims 1 to 4, wherein the method reduces IL-23, IL-12b, and/or IL-17 RNA levels in skin of the subject.

6. The method of any one of claims 1 to 5, wherein the skin comprises a lesion, such as a lesion associated with psoriasis.

7. A method of reducing IL-1b, IL-2, IFNg, IL-4, IL-5, IL-6, IL-8, IL-10, TNFa and/or IL-17 protein levels in a subject in need thereof, comprising orally administering to the subject a bacterial composition comprising a Prevotella histicola strain comprising at least 99% genomic, 16S and/or CRISPR sequence identity to the nucleotide sequence of the Prevotella Strain B 50329 (NRRL accession number B 50329).

8. The method of claim 7, wherein the method reduces IL-1b, IL-2, IFNg, IL-4, IL-5, IL-6, IL-8, IL-10, TNFa and/or IL-17 protein levels in blood (such as in peripheral blood mononuclear cells (PBMCs)) of the subject.

9. A method of deepening a response in a post-dosing period in a subject in need thereof, comprising administering to the subject a bacterial composition comprising a Prevotella histicola strain comprising at least 99% genomic, 16S and/or CRISPR sequence identity to the nucleotide sequence of the Prevotella Strain B 50329 (NRRL accession number B 50329) prior to the post-dosing period, wherein a deepening of response is determined by improvement in the Psoriasis Area and Severity Index (PASI) score (e.g., as described herein).

10. The method of claim 9, wherein the deepening of response is in the 8, 12, 16, 20, 24, or 28 week post-dosing period.

11. A method of maintaining a PASI-50 or greater (e.g., as described herein) in a post-dosing period in a subject in need thereof, comprising administering to the subject a bacterial composition comprising a Prevotella histicola strain comprising at least 99% genomic, 16S and/or CRISPR sequence identity to the nucleotide sequence of the Prevotella Strain B 50329 (NRRL accession number B 50329) prior to the post-dosing period.

12. The method of claim 11, wherein the maintaining a PASI-50 or greater is in the 8, 12, 16, 20, 24, or 28 week post-dosing period.

13. A method of decreasing the likelihood of rebound in a post-dosing period in a subject in need thereof, comprising administering to the subject a bacterial composition comprising a Prevotella histicola strain comprising at least 99% genomic, 16S and/or CRISPR sequence identity to the nucleotide sequence of the Prevotella Strain B 50329 (NRRL accession number B 50329) prior to the post-dosing period.

14. The method of any one of claims 9 to 13, wherein the Prevotella histicola strain is administered for 16 weeks prior to the post-dosing period.

15. The method of any one of claims 9 to 14, wherein the subject is a responder, wherein a responder is a subject achieved at least a 50% reduction in their Psoriasis Area and Severity Index (PASI) scores from baseline at week 16 after administration of the Prevotella histicola strain.

16. The method of any one of claims 1 to 15, wherein the Prevotella histicola strain is Prevotella Strain B 50329 (NRRL accession number B 50329).

17. The method of any one of claims 1 to 16, wherein the bacterial composition is formulated in one or more capsules or tablets, optionally wherein the capsules or tablets are enteric coated.

18. The method of any one of claims 1 to 17, wherein the bacterial composition comprises about 8×10¹⁰ to about 8×10¹¹ total cells of Prevotella histicola Strain B 50329 (NRRL accession number B 50329), optionally formulated in one or more capsules or tablets, optionally wherein the capsules or tablets are enteric coated.

19. The method of any one of claims 1 to 18, wherein the bacterial composition comprises about 0.8×10¹¹ total cells of Prevotella histicola Strain B 50329 (NRRL accession number B 50329), optionally formulated in one or more capsules or tablets, optionally wherein the capsules or tablets are enteric coated.

20. The method of any one of claims 1 to 18, wherein the bacterial composition comprises about 3.2×10¹¹ total cells of Prevotella histicola. Strain B 50329 (NRRL accession number B 50329), optionally formulated in one or more capsules or tablets, optionally wherein the capsules or tablets are enteric coated.

21. The method of any one of claims 1 to 18, wherein the bacterial composition comprises about 8.0×10¹¹ total cells of Prevotella histicola Strain B 50329 (NRRL accession number B 50329), optionally formulated in one or more capsules or tablets, optionally wherein the capsules or tablets are enteric coated.

22. The method of any one of claims 1 to 21, wherein the bacterial composition is administered at least once daily.

23. The method of any one of claims 1 to 21, wherein the bacterial composition is administered once daily.

24. The method of claim 23, wherein the bacterial composition is administered once daily for at least 16 weeks.

25. The method of any one of claims 1 to 24, wherein the bacterial composition is administered twice daily.

26. The method of claim 25, wherein the bacterial composition is administered twice daily for at least 16 weeks.

27. The method of any one of claims 1 to 26 wherein the subject has inflammation.

28. The method of any one of claims 1 to 27 wherein the subject has Th1-mediated inflammation.

29. The method of any one of claims 1 to 27 wherein the subject has Th2-mediated inflammation.

30. The method of any one of claims 1 to 27 wherein the subject has Th17-mediated inflammation.

31. The method of any one of claims 1 to 30 wherein the subject is a human.
